(19) United States
(12) Patent Application Publication
(10) Pub. No.: US 2025/0041307 A1
(43) Pub. Date: Feb. 6, 2025

(54) Uses of Bicyclic Compounds for the Treatment of Diseases

(71) Applicant: Athira Pharma, Inc., Bothell, WA (US)

(72) Inventors: Leen Kawas, Lynnwood, WA (US); Kevin Church, Mountlake Terrace, WA (US); Robert Taylor, Seattle, WA (US); Jewel Johnston, Seattle, WA (US); Douglas Boatman, Portland, OR (US)

(73) Assignee: Athira Pharma, Inc., Bothell, WA (US)

(21) Appl. No.: 18/716,404

(22) PCT Filed: Dec. 16, 2022
(86) PCT No.: PCT/US2022/053113

§ 371 Date: Jun. 4, 2024

Related U.S. Application Data
(60) Provisional application No. 63/312,696, filed on Feb. 22, 2022.
(60) Provisional application No. 63/290,783, filed on Dec. 17, 2021.

(51) Int. Cl. A61K 31/519

(52) U.S. Cl. CPC A61K 31/519

(57) ABSTRACT

Provided herein are compounds and compositions thereof for modulating hepatocyte growth factors. In some embodiments, the compounds and compositions are provided for treatment of diseases, including neurological disorders.

### FIELD

The present disclosure relates generally to use of compounds and compositions for treating diseases, such as mild cognitive impairment.

### BACKGROUND

Hepatocyte growth factor (HGF) is a pleiotropic protein factor involved in numerous biological processes including embryonic and organ development, regeneration, and inflammation. HGF is a critical contributor to cortical, motor, sensory, sympathetic, and parasympathetic neuronal development and maturation. HGF is translated and secreted as inactive pro-HGF, but following cleavage, the resultant α and β-subunits are joined by a disulfide linkage to form the active heterodimer. Expression of HGF predominantly occurs in mesenchymal cells such as fibroblasts, chondroblasts, adipocytes, and the endothelium. Expression has also been demonstrated in the central nervous system (CNS) including neurons, astrocytes, and ependymal cells (Nakamura and Mizuno, 2010). All biological activities of HGF are mediated through MET, a transmembrane receptor tyrosine kinase that serves as the sole known receptor for HGF. MET has known involvement in a variety of biological processes, with demonstrated roles in development, regeneration, and response to injury. Upon binding of HGF to the extracellular domain of MET, homo-dimerization of the MET protein leads to auto-phosphorylation of the intracellular domain. Phosphorylation of MET intracellular domains leads to recruitment and phosphorylation of a variety of effector proteins including Gab1, GRB2, Phospholipase C, and Stat3 (Gherardi et al., 2012; Organ and Tsao, 2011). These effector proteins then interact with downstream signaling pathways including PI3K/Akt, Ras/Raf/MAPK, RAC1/CDC42, RAP/FAK among others to influence an array of cellular components including gene regulation, cytoskeletal rearrangements, cell cycle progression, cell adhesion, survival, and proliferation (Organ and Tsao, 2011).

Because HGF has a demonstrated role in development (Nakamura et al., 2011), homeostasis (Funakoshi and Nakamura, 2003), suppression of cell death, and regeneration (Matsumoto et al., 2014), stimulation of the HGF/MET signaling system is an ideal target for therapeutics for a range of disease states. Therapeutics involving HGF activity modulation have been proposed for disease and injury in many diverse tissue types including liver, kidney, gastrointestinal tract, cardiovascular components, lung, skin, nervous system, and musculature (Matsumoto et al., 2014). However, highly efficacious compounds useful for the modulation of HGF/MET signaling activity are yet to be explored and discovered.

Although progress has been made in this field, there remains a need for improved compounds and methods for treatment of HGF-modulated diseases. Accordingly, in one aspect, provided herein are compounds which modulate HGF for use in treating neurodegenerative diseases.

### SUMMARY

Described herein, in certain embodiments, are compounds and compositions thereof for modulating hepatocyte growth factor (HGF) for treatment of diseases. Nonlimiting exemplary embodiments include:

Embodiment 1. A method of treating mild cognitive impairment in a subject in need thereof, comprising administering an effective amount of a compound of Formula (I):

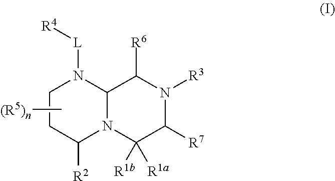

or a pharmaceutically acceptable salt thereof, wherein:
L is a direct bond, —C(═O)—, —(CR^{a}R^{b})ₘ—C(═O)—, —C(═O)—(CR^{a}R^{b})ₘ—, or —(CR^{a}R^{b})ₘ—;
each R^{a} and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
   wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
   wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

Embodiment 2. The method of embodiment 1, wherein L is —C(═O)— or —(CR^{a}R^{b})ₘ—.

Embodiment 3. The method of embodiment 1 or 2, wherein L is a —C(═O)—.

Embodiment 4. The method of embodiment 1 or 2, wherein L is —(CR^{a}R^{b})ₘ—.

Embodiment 5. The method of embodiment 4, wherein R^{a} and R^{b} are each H, and m is 1.

Embodiment 6. The method of any one of embodiments 1-5, wherein R^{1a} and R^{1b} are each independently H; C₁-C₆ alkyl optionally substituted with 1-3 substituents selected from halo, —CO₂H, and —C(═O)NH₂; C₁-C₆ alkoxy; halo; or C₆-C₁₀ arylalkyl optionally substituted by 1-3 substituents selected from halo and amino.

Embodiment 7. The method of embodiment 6, wherein R^{1a} and R^{1b} are each independently H, methyl, fluoro, 2-methylbutyl, —CH₂F, methoxy, —CH₂CO₂H, —CH₂C(═O)NH₂, benzyl, or 4-aminobenzyl.

Embodiment 8. The method of embodiment 6, wherein R^{1a} and R^{1b} are each independently H or C₁-C₃ alkyl.

Embodiment 9. The method of embodiment 8, wherein R^{1a} is methyl and R^{1b} is H.

Embodiment 10. The method of embodiment 8, wherein R^{1a} and R^{1b} are each H.

Embodiment 11. The method of any one of embodiments 1-10, wherein R² is H.

Embodiment 12. The method of any one of embodiments 1-10, wherein R² is thioxo.

Embodiment 13. The method of any one of embodiments 1-10, wherein R² is oxo.

Embodiment 14. The method of any one of embodiments 1-13, wherein R³ is C₃-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl, wherein the alkyl, alkenyl, alkynyl, cycloalkyl, cycloalkylalkyl, arylalkyl, heteroarylalkyl, or heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

Embodiment 15. The method of any one of embodiments 1-13, wherein R³ is C₂-C₆ alkyl optionally substituted by 1-3 substituents selected from halo, C₁-C₃ alkoxy, hydroxy, —NH₂, —SO₂(C₁-C₃ alkyl), and —C(═O)NH₂; C₂-C₆ alkenyl; C₃-C₆ cycloalkylalkyl; 5- to 6-membered heteroarylalkyl; 5- to 6-membered heterocyclylalkyl; or C₆ arylalkyl.

Embodiment 16. The method of embodiment 15, wherein R³ is C₂ alkyl substituted by 1-3 substituents selected from C₁-C₃ alkoxy, hydroxy, —NH₂, and —SO₂(C₁-C₃ alkyl).

Embodiment 17. The method of any one of embodiments 14-16, wherein R³ is:

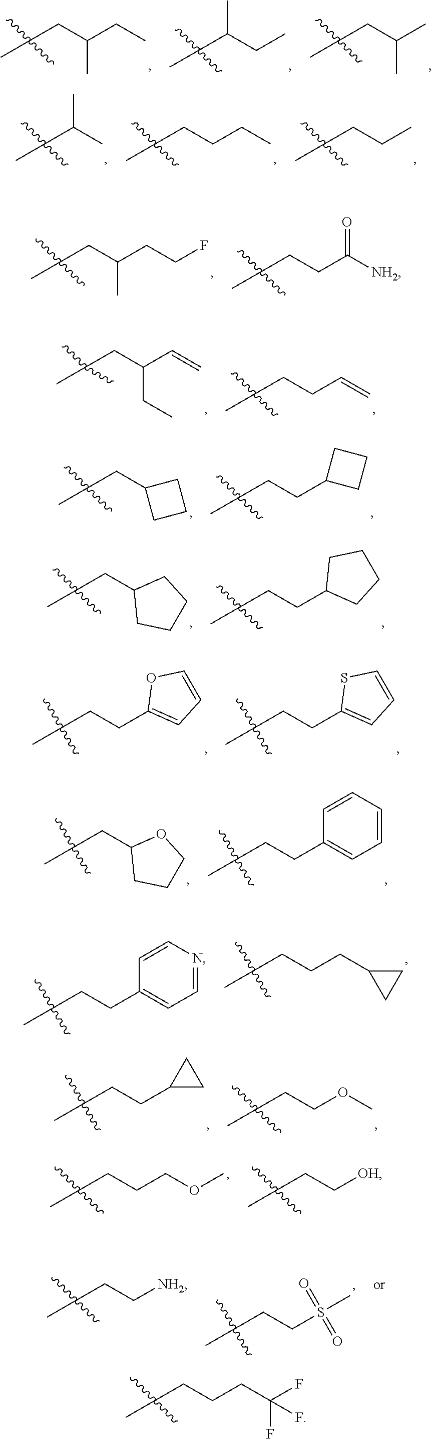

Embodiment 18. The method of embodiment 17, wherein R³ is:

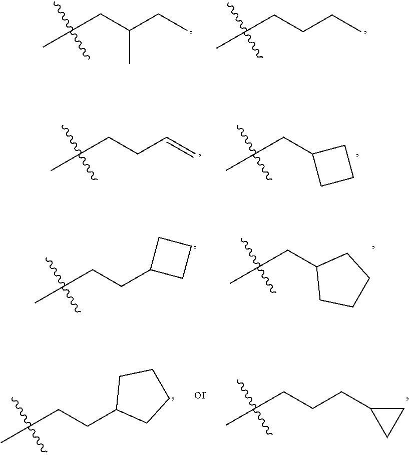

Embodiment 19. The method of any one of embodiments 1-18, wherein R⁴ is C₆-C₁₀ aryl optionally substituted with 1-3 substituents selected from halo, hydroxyl, C₁-C₆ haloalkyl, and C₁-C₆ haloalkoxy.

Embodiment 20. The method of embodiment 19, wherein R⁴ is phenyl substituted with 1-3 substituents selected from —CF₃, —OCHF₂, —OH, fluoro, and chloro.

Embodiment 21. The method of embodiment 20, wherein R⁴ is:

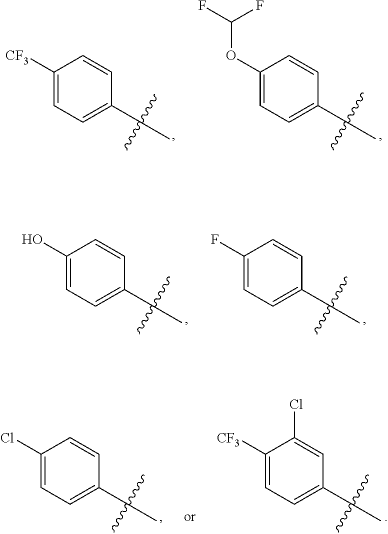 [SMILES: *c1ccc(C(F)(F)F)cc1 ; *c1ccc(OC(F)F)cc1 ; *c1ccc(O)cc1 ; *c1ccc(F)cc1 ; *c1ccc(Cl)cc1 ; *c1ccc(C(F)(F)F)c(Cl)c1]

Embodiment 22. The method of embodiment 21, wherein R⁴ is:

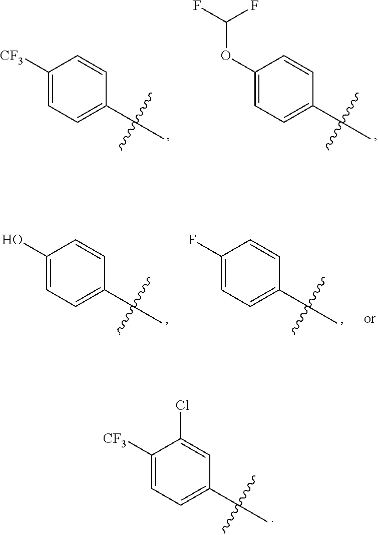 [SMILES: *c1ccc(C(F)(F)F)cc1 ; *c1ccc(OC(F)F)cc1 ; *c1ccc(O)cc1 ; *c1ccc(F)cc1 ; *c1ccc(C(F)(F)F)c(Cl)c1]

Embodiment 23. The method of any one of embodiments 1-18, wherein R⁴ is 5- to 10-membered heteroaryl optionally substituted with 1-3 substituents selected from halo, hydroxyl, C₁-C₆ haloalkyl, and C₁-C₆ haloalkoxy.

Embodiment 24. The method of embodiment 23, wherein R⁴ is pyridyl or indolyl optionally substituted with 1-3 substituents selected from halo, hydroxyl, C₁-C₆ haloalkyl, and C₁-C₆ haloalkoxy.

Embodiment 25. The method of embodiment 24, wherein

Embodiment 26. The method of embodiment 25, wherein
R⁴ is

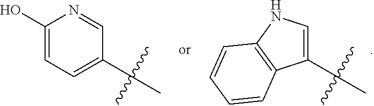 [SMILES: *c1ccc(O)nc1 ; *c1c[nH]c2ccccc12]

Embodiment 27. The method of any one of embodiments 1-18, wherein R⁴ is 5- to 10-membered heterocyclyl optionally substituted with 1-3 substituents selected from halo, hydroxyl, C₁-C₆ haloalkyl, and C₁-C₆ haloalkoxy.

Embodiment 28. The method of embodiment 27, wherein R⁴ is indolinyl.

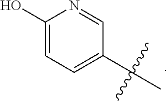 [SMILES: *c1ccc(O)nc1]

Embodiment 29. The method of embodiment 28, wherein R⁴ is

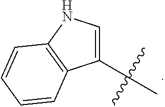 [SMILES: *c1c[nH]c2ccccc12]

Embodiment 30. The method of any one of embodiments 1-26, wherein -L-R⁴ is:

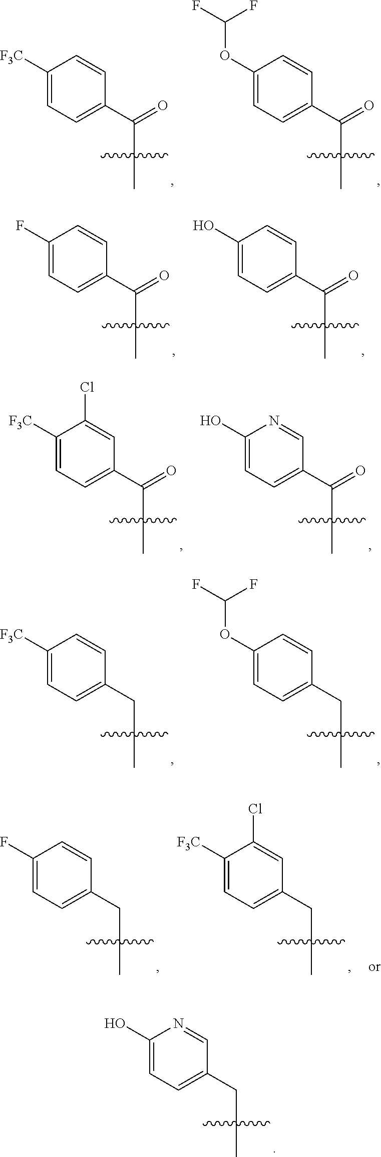

Embodiment 31. The method of any one of embodiments 1-30, wherein n is 0.

Embodiment 32. The method of any one of embodiments 1-30, wherein n is 1.

Embodiment 33. The method of embodiment 32, wherein R⁵ is oxo or halo.

Embodiment 34. The method of embodiment 33, wherein R⁵ is oxo or fluoro.

Embodiment 35. The method of any one of embodiments 1-34, wherein R⁶ is H.

Embodiment 36. The method of any one of embodiments 1-35, wherein R⁷ is oxo.

Embodiment 37. The method of any one of embodiments 1-10, 13-31, 35, and 36, wherein the compound is of Formula (V):

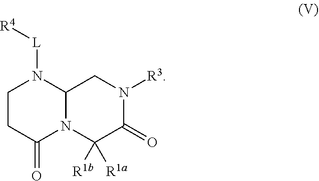

Embodiment 38. The method of embodiment 37, wherein:
L is —C(═O)— or —CH₂—;
R^{1a} and R^{1b} are independently H or C₁-C₃ alkyl optionally substituted with —CO₂H;
R³ is C₄-C₅ alkyl, C₄-C₅ alkenyl, or C₁-C₃ alkyl substituted with C₃-C₅ cycloalkyl; and
R⁴ is phenyl or pyridyl substituted with 1-3 substituents selected from —CF₃, —OCHF₂, —OH, fluoro, and chloro.

Embodiment 39. A method of treating mild cognitive impairment in a subject in need thereof, comprising administering an effective amount of a compound selected from the compounds of Table 1A and pharmaceutically acceptable salts thereof.

Embodiment 40. The method of any one of the preceding claims, wherein the method slows progression of dementia in the subject.

Embodiment 41. The method of any one of the preceding claims, wherein the method improves cognitive function and/or slows progression of cognitive dysfunction in the subject.

### DETAILED DESCRIPTION

### Definitions

Unless defined otherwise, all technical and scientific terms used herein have the same meaning as is commonly understood by one of skill in the art to which this disclosure belongs. In the following description, certain specific details are set forth in order to provide a thorough understanding of various embodiments of the disclosure. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the —NO₂ radical.

“Oxo” refers to the ═O substituent.

“Thioxo” refers to the ═S substituent.

“Thiol” refers to the —SH substituent.

“Alkyl” refers to an unbranched or branched saturated hydrocarbon chain radical consisting solely of carbon and hydrogen atoms, having from one to twelve carbon atoms (C₁-C₁₂ alkyl), preferably one to eight carbon atoms (C₁-C₈ alkyl), one to six carbon atoms (C₁-C₆ alkyl), or one to three carbon atoms (C₁-C₃ alkyl) and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. Unless stated otherwise specifically in the specification, an alkyl group is optionally substituted.

“Alkenyl” refers to an unbranched or branched unsaturated hydrocarbon chain radical consisting solely of carbon and hydrogen atoms, which contains one or more carbon-carbon double bonds, having from two to twelve carbon atoms (C₂-C₁₂ alkenyl), preferably two to eight carbon atoms (C₂-C₈ alkenyl) or two to six carbon atoms (C₂-C₆ alkenyl), and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group is optionally substituted.

“Alkynyl” refers to an unbranched or branched unsaturated hydrocarbon chain radical consisting solely of carbon and hydrogen atoms, which contains one or more carbon-carbon triple bonds, having from two to twelve carbon atoms (C₂-C₁₂ alkynyl), preferably two to eight carbon atoms (C₂-C₈ alkynyl) or two to six carbon atoms (C₂-C₆ alkynyl), and which is attached to the rest of the molecule by a single bond, e.g., ethynyl, propynyl, butynyl, pentynyl, hexynyl, and the like. Unless stated otherwise specifically in the specification, an alkynyl group is optionally substituted.

“Alkoxy” refers to a radical of the formula —ORₐ where Rₐ is an alkyl radical as defined above containing one to twelve carbon atoms. Preferred alkoxy groups have one to six carbon atoms (i.e., C₁-C₆ alkoxy) or one to three carbon atoms (i.e., C₁-C₃ alkoxy) in the alkyl radical. Unless stated otherwise specifically in the specification, an alkoxy group is optionally substituted.

“Aromatic ring” refers to a cyclic planar portion of a molecule (i.e., a radical) with a ring of resonance bonds that exhibits increased stability relative to other connective arrangements with the same sets of atoms. Generally, aromatic rings contain a set of covalently bound co-planar atoms and comprise a number of 7-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an aromatic ring includes all radicals that are optionally substituted.

“Aryl” refers to a carbocyclic ring system radical comprising 6 to 18 carbon atoms and at least one aromatic ring (i.e., C₆-C₁₅ aryl), preferably having 6 to 10 carbon atoms (i.e., C₆-C₁₀ aryl). For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, phenyl, pleiadene, pyrene, and triphenylene. Unless stated otherwise specifically in the specification, an aryl group is optionally substituted.

“Arylalkyl” refers to a radical of the formula —R_{b}—R_{c}, where R_{b} is an alkylene chain and R, is one or more aryl radicals as defined above, for example, benzyl, diphenylmethyl and the like. An arylalkyl group may contain a C₁-C₁₀ alkylene chain connected to a C₆-C₁₀ aryl radical (i.e., C₆-C₁₀ arylalkyl). Unless stated otherwise specifically in the specification, an arylalkyl group is optionally substituted.

“Cycloalkyl” refers to a stable non-aromatic monocyclic or polycyclic carbocyclic radical consisting solely of carbon and hydrogen atoms, which may include fused or bridged ring systems, having from three to fifteen carbon atoms (i.e., C₃-C₁₅ cycloalkyl), preferably having from three to ten carbon atoms (i.e., C₃-C₁₀ cycloalkyl) or three to six carbon atoms (i.e., C₃-C₆ cycloalkyl), and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Cycloalkyl also includes “spiro cycloalkyl” when there are two positions for substitution on the same carbon atom. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Cycloalkylalkyl” refers to a radical of the formula —R_{b}—R_{c} where R_{b} is an alkylene chain and R, is one or more cycloalkyl radicals as defined above, for example, cyclopropyl, cyclobutyl, cyclopentyl, cyclohexyl, cycloheptyl, and cyclooctyl and the like. A cycloalkylalkyl group may contain a C₁-C₁₀ alkylene chain connected to a C₃-C₁₂ cycloalkyl radical (i.e., C₃-C₁₂ cycloalkylalkyl) or a C₁-C₁₀ alkylene chain connected to a C₃-C₆ cycloalkyl radical (i.e., C₃-C₆ cycloalkylalkyl). Unless stated otherwise specifically in the specification, a cycloalkylalkyl group is optionally substituted.

“Fused” refers to any ring structure described herein which is fused to an existing ring structure in the compounds of the disclosure. When the fused ring is a heterocyclyl ring or a heteroaryl ring, any carbon atom on the existing ring structure which becomes part of the fused heterocyclyl ring or the fused heteroaryl ring is replaced with a nitrogen atom.

“Halo” or “halogen” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A preferred haloalkyl group includes an alkyl group having one to six carbon atoms and that is substituted by one or more halo radicals (i.e., C₁-C₆ haloalkyl). The halo radicals may be all the same or the halo radicals may be different. Unless stated otherwise specifically in the specification, a haloalkyl group is optionally substituted.

“Haloalkoxy” refers to a radical of the formula —ORₐ where Rₐ is a haloalkyl radical as defined herein containing one to twelve carbon atoms. A preferred haloalkoxy group includes an alkoxy group having one to six carbon atoms (i.e., C₁-C₆ haloalkoxy) or having one to three carbon atoms (C₁-C₃ haloalkoxy) and that is substituted by one or more halo radicals. The halo radicals may all be the same or the halo radicals may all be different. Unless stated otherwise specifically in the specification, a haloalkoxy group is optionally substituted.

“Heteroaryl” refers to an aromatic group (e.g., a 5-14 membered ring system) having a single ring, multiple rings, or multiple fused rings, with one or more ring heteroatoms independently selected from nitrogen, oxygen and sulfur. As used herein, heteroaryl includes 1 to 10 ring carbon atoms and 1 to 4 heteroatoms independently selected from nitrogen, oxygen and sulfur within the ring. Preferred heteroaryl groups have a 5- to 10-membered ring system containing one to four heteroatoms selected from nitrogen, oxygen, and sulfur (i.e., a 5- to 10-membered heteroaryl) and a 5- to 6-membered ring system containing one to four heteroatoms selected from nitrogen, oxygen, and sulfur (i.e., a 5- to 6-membered heteroaryl). For purposes of embodiments of this disclosure, the heteroaryl radical may be a monocyclic, bicyclic, tricyclic or tetracyclic ring system, which may include fused or bridged ring systems. Examples of heteroaryl groups include pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl). A heteroaryl may comprise one or more N-oxide (N—O—) moieties, such as pyridine-N-oxide. Unless stated otherwise specifically in the specification, a heteroaryl group is optionally substituted.

“Heteroarylalkyl” refers to a radical of the formula —R_{b}—R_{c} where R_{b} is an alkylene chain and R, is one or more heteroaryl radicals as defined above. A heteroarylalkyl group may contain a C₁-C₁₀ alkylene chain connected to a 5- to 10-membered heteroaryl group (i.e., 5- to 10-membered heteroarylalkyl) or a C₁-C₁₀ alkylene chain connected to a 5- to 6-membered heteroaryl group (i.e., 5- to 6-membered heteroarylalkyl). Unless stated otherwise specifically in the specification, a heteroarylalkyl group is optionally substituted.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (C═O) or N-oxide (N—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 1 to 10 ring carbon atoms, 1 to 8 carbon atoms, 1 to 6 carbon atoms, or 1 to 4 carbon atoms, and 1 to 5 ring heteroatoms, 1 to 4 heteroatoms, 1 to 3 heteroatoms, or 1 to 2 heteroatoms independently selected from nitrogen, sulfur and oxygen. Preferred heterocyclyls have five to 10 members in the ring system including one to four heteroatoms selected from nitrogen and oxygen (i.e., 5- to 10-membered heterocyclyl) or five to eight members in the ring system including one to four heteroatoms selected from nitrogen and oxygen (i.e., 5- to 8-membered heterocyclyl). Examples of heterocyclyl groups include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl and 1,1-dioxo-thiomorpholinyl. Unless stated otherwise specifically in the specification, a hetercyclyl group is optionally substituted.

“Heterocyclylalkyl” refers to a radical of the formula —R_{b}—R_{c} where R_{b} is an alkylene chain and R, is one or more heterocyclyl radicals as defined above. A heterocyclylalkyl group may contain a C₁-C₁₀ alkylene chain connected to a 5- to 10-membered heterocyclyl radical (i.e., 5- to 10-membered heterocyclylalkyl) or a C₁-C₁₀ alkylene chain connected to a 5- to 8-membered heterocyclyl radical (i.e., 5- to 8-membered heterocyclylalkyl). Unless stated otherwise specifically in the specification, a heterocyclylalkyl group is optionally substituted.

In some embodiments, the term “substituted” as used herein means any of the above groups, or other substituents (e.g., C₁-C₆ alkyl, C₂-C₆ alkenyl, C₂-C₆ alkynyl, C₃-C₁₂ cycloalkyl, C₃-C₁₂ cycloalkylalkyl, aryl, and heteroaryl) wherein at least one hydrogen atom (e.g., 1, 2, 3, or all hydrogen atoms) is replaced by a bond to a non-hydrogen atom such as, but not limited to: a halogen atom such as F, Cl, Br, and I (i.e., “halo”); an oxygen atom in groups such as hydroxyl groups or alkoxy groups (e.g., alkoxy or haloalkoxy); a nitrogen atom in groups such as amines (e.g., —NH₂), amides (e.g., —(C═O)NH₂), and nitro; alkyl groups including one or more halogen, such as F, Cl, Br, and I (e.g., haloalkyl); and cyano.

It is understood that each choice for L, R^{1a}, R^{1b}, R², R³, R⁴, R⁵, R⁶, and R⁷ is optionally substituted as described above unless specifically stated otherwise, and provided that all valences are satisfied by the substitution. Specifically, each choice for L, R^{1a}, R^{1b}, R², R³, R⁴, R⁵, R⁶, and R⁷ is optionally substituted unless specifically stated otherwise, and provided such substitution results in a stable molecule (e.g., groups such as H and halo are not optionally substituted).

“Mild cognitive impairment” and “MCI,” as used herein, refer to cognitive decline that falls between cognitive changes of aging and early dementia. Individuals exhibiting MCI show evidence of cognitive impairment that represents a decline from the past, but are able to function substantially independently in their daily lives. Mild cognitive impairment may be a precursor to dementia, but involves cognitive impairment that is not severe enough to be classified as dementia. MCI may impact one or more cognitive functions, including but not limited to, learning and memory, language, visuo-spatial skills, executive functioning, and psychomotor functioning. See, e.g., Knopman et al., 2014, Mayo Clin Proc, 89(10): 1452-1459.

“Effective amount” or “therapeutically effective amount” of a compound or a composition refers to that amount of the compound or the composition that results in an intended result as desired based on the disclosure herein. Effective amounts can be determined by standard pharmaceutical procedures in cell cultures or experimental animals including, without limitation, by determining the ED₅₀ (the dose therapeutically effective in 50% of the population) and the LD₅₀ (the dose lethal to 50% of the population). In some embodiments, an effective amount of a compound results in reduction or inhibition of symptoms or a prolongation of survival in a subject (i.e., a human patient). The results may require multiple doses of the compound.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the invention contemplate any one or more of these aspects of treatment.

As used herein, the terms “individual(s)”, “subject(s)” and “patient(s)” mean any mammal. Examples include, but are not limited to, mice, rats, hamsters, guinea pigs, pigs, rabbits, cats, dogs, goats, sheep, cows, and humans. In some embodiments, the mammal is a human.

A “therapeutic effect”, as that term is used herein, encompasses a therapeutic benefit and/or a prophylactic benefit as described herein. A therapeutic effect includes delaying or eliminating the appearance of a disease or condition; delaying or eliminating the onset of symptoms of a disease or condition; slowing, halting, or reversing the progression of a disease or condition; causing partial or complete regression of a disease or condition; or any combination thereof.

The terms “co-administration”, “administered in combination with”, and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine.

In some embodiments, pharmaceutically acceptable salts include quaternary ammonium salts such as quaternary amine alkyl halide salts (e.g., methyl bromide).

As used herein, “therapeutic agent” refers to a biological, pharmaceutical, or chemical compound or other moiety. Non-limiting examples include a simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like.

The term “in vivo” refers to an event that takes place in a subject's body.

Embodiments of the disclosure are also meant to encompass all pharmaceutically acceptable compounds of Formula (I) being isotopically-labelled by having one or more atoms replaced by an atom having a different atomic mass or mass number (i.e., an “isotopic form” of a compound of Formula (I)). Examples of isotopes that can be incorporated into the compounds of Formula (I) include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled compounds of Formula (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence are preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I) can generally be prepared by conventional techniques known to those skilled in the art or by processes analogous to those described in the Examples as set out below using an appropriate isotopically-labeled reagent in place of the non-labeled reagent previously employed.

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compound of the disclosure. As used herein, the term “solvate” refers to an aggregate that comprises one or more molecules of a compound of Formula (I) with one or more molecules of solvent. In some embodiments, the solvent is water, in which case the solvate is a hydrate. Alternatively, in other embodiments, the solvent is an organic solvent. Thus, the compounds of Formula (I) may exist as a hydrate, including a monohydrate, dihydrate, hemihydrate, sesquihydrate, trihydrate, tetrahydrate and the like, as well as the corresponding solvated forms. In some aspects, the compound of Formula (I) is a true solvate, while in other cases, the compound of the disclosure merely retains adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan.

A “pharmaceutical composition” or “pharmaceutically acceptable composition” refers to a formulation of a compound of the disclosure and a medium generally accepted in the art for the delivery of the biologically active compound to mammals, e.g., humans. Such a medium includes all pharmaceutically acceptable carriers, diluents, or excipients therefor.

“Pharmaceutically acceptable carrier, diluent or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The compounds of Formula (I), or a pharmaceutically acceptable salt or isotopic form thereof, may contain one or more centers giving rise to geometric asymmetry and may thus provide enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

“Diastereoisomers” are stereoisomers that have at least two asymmetric atoms, but which are not mirror-images of each other.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Ultra Version 11.0.1 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

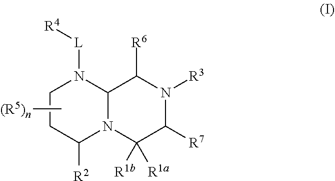

or a pharmaceutically acceptable salt thereof, wherein:
L is a direct bond, —C(═O)—, —(CR^{a}R^{b})ₘ—C(═O)—, —C(═O)—(CR^{a}R^{b})ₘ—, or —(CR^{a}R^{b})ₘ—;
each Rₐ and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
   wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
   wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

In some embodiments, L is a direct bond. In some embodiments, L is —C(═O)— or —(CR^{a}R^{b})ₘ—. In some embodiments, L is —C(═O)—. In some embodiments, L is —(CR^{a}R^{b})ₘ—. In some embodiments, L is —(CR^{a}R^{b})ₘ—C(═O)— or —C(═O)—(CR^{a}R^{b})ₘ—. In some embodiments, L is —(CR^{a}R^{b})ₘ—C(═O)—. In some embodiments, L is —C(═O)—(CR^{a}R^{b})ₘ—.

In some embodiments, each Rₐ and R^{b} is independently H, C₁-C₆ alkyl, C₂-C₆ alkenyl, or C₂-C₆ alkynyl. In some embodiments, each Rₐ and R^{b} is independently H, C₁-C₃ alkyl, C₂-C₄ alkenyl, or C₂-C₄ alkynyl. In some embodiments, Rₐ and R^{b} are each H. In some embodiments, Rₐ is H. In some embodiments, Rₐ is C₁-C₆ alkyl, such as methyl, ethyl, or propyl. In some embodiments, Rₐ is C₂-C₆ alkenyl, such as vinyl or propenyl. In some embodiments, Rₐ is C₂-C₆ alkynyl, such as ethynyl or propynyl. In some embodiments, R^{b} is H. In some embodiments, R^{b} is C₁-C₆ alkyl, such as methyl, ethyl, or propyl. In some embodiments, R^{b} is C₂-C₆ alkenyl, such as vinyl or propenyl. In some embodiments, R^{b} is C₂-C₆ alkynyl, such as ethynyl or propynyl.

In some embodiments, R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl. In some embodiments, R^{1a} is H. In some embodiments, R^{1a} is C₁-C₆ alkyl, such as methyl, ethyl, or propyl. In some embodiments, R^{1a} is C₂-C₆ alkenyl, such as vinyl or propenyl. In some embodiments, R^{1a} is C₂-C₆ alkynyl, such as ethynyl or propynyl. In some embodiments, R^{1a} is C₁-C₆ alkoxy, such as methoxy, ethoxy, or propoxy. In some embodiments, R^{1a} is halo, such as fluoro, chloro, or bromo. In some embodiments, R^{1a} is C₆-C₁₀ arylalkyl, such as benzyl. In some embodiments, R^{1b} is H. In some embodiments, R^{1b} is C₁-C₆ alkyl, such as methyl, ethyl, or propyl. In some embodiments, R^{1b} is C₂-C₆ alkenyl, such as vinyl or propenyl. In some embodiments, R^{1b} is C₂-C₆ alkynyl, such as ethynyl or propynyl. In some embodiments, R^{1b} is C₁-C₆ alkoxy, such as methoxy, ethoxy, or propoxy. In some embodiments, R^{1b} is halo, such as fluoro, chloro, or bromo. In some embodiments, R^{1b} is C₆-C₁₀ arylalkyl, such as benzyl.

In some embodiments, R^{1a} and R^{1b} are each independently H; C₁-C₆ alkyl optionally substituted with 1-3 substituents selected from halo, —CO₂H, and —C(═O)NH₂; C₁-C₆ alkoxy; halo; or C₆-C₁₀ arylalkyl optionally substituted by 1-3 substituents selected from halo and amino. In some embodiments, R^{1a} is C₁-C₆ alkyl substituted with 1-3 halo, such as fluoro or chloro. In some embodiments, R^{1a} is C₁-C₆ alkyl substituted with 1-3 —CO₂H groups. In some variations, R^{1a} is C₁-C₃ alkyl substituted with 1-2 CO₂H groups, such as —CH₂CO₂H or —CH₂CH₂CO₂H. In some embodiments, R^{1a} is C₁-C₆ alkyl substituted with 1-3 —C(═O)NH₂ groups. In some embodiments, R^{1a} is C₁-C₃ alkyl substituted with 1-2 —C(═O)NH₂ groups, such as —CH₂C(═O)NH₂ or —CH₂CH₂C(═O)NH₂. In some embodiments, R^{1a} is C₆-C₁₀ arylalkyl substituted by 1-3 substituents selected from halo and amino. In some embodiments, R^{1a} is C₆-C₁₀ arylalkyl substituted by 1-3 halo, such as fluoro, chloro, or bromo. In some embodiments, R^{1a} is C₆-C₁₀ arylalkyl substituted by 1-3 amino. In some embodiments, R^{1b} is C₁-C₆ alkyl substituted with 1-3 halo, such as fluoro or chloro. In some embodiments, R^{1b} is C₁-C₆ alkyl substituted with 1-3 —CO₂H groups. In some variations, R^{1b} is C₁-C₃ alkyl substituted with 1-2 CO₂H groups, such as —CH₂CO₂H or —CH₂CH₂CO₂H. In some embodiments, R^{1b} is C₁-C₆ alkyl substituted with 1-3 —C(═O)NH₂ groups. In some embodiments, R^{1b} is C₁-C₃ alkyl substituted with 1-2 —C(═O)NH₂ groups, such as —CH₂C(═O)NH₂ or —CH₂CH₂C(═O)NH₂. In some embodiments, R^{1b} is C₆-C₁₀ arylalkyl substituted by 1-3 substituents selected from halo and amino. In some embodiments, R^{1b} is C₆-C₁₀ arylalkyl substituted by 1-3 halo, such as fluoro, chloro, or bromo. In some embodiments, R^{1b} is C₆-C₁₀ arylalkyl substituted by 1-3 amino. In some embodiments, R^{1a} and R^{1b} are each independently H, methyl, fluoro, 2-methylbutyl, —CH₂F, methoxy, —CH₂CO₂H, —CH₂C(═O)NH₂, benzyl, or 4-aminobenzyl. In some embodiments, R^{1a} and R^{1b} are each independently H or C₁-C₃ alkyl. In some embodiments, R^{1a} is methyl and R^{1b} is H. In some embodiments, R^{1a} and R^{1b} are each H. In some embodiments, one of R^{1a} and R^{1b} is H and the other is C₁-C₃ alkyl, such as methyl.

In some embodiments, R² is H, oxo, or thioxo. In some embodiments, R² is H. In some embodiments, R² is oxo. In some embodiments, R² is thioxo.

In some embodiments, R³ is C₃-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl, wherein the 5- to 10-membered heteroarylalkyl or 5- to 10-membered heterocyclylalkyl contains 1-3 heteroatoms selected from nitrogen and oxygen. In some embodiments, R³ is C₃-C₆ alkyl, such as propyl, butyl, pentyl, or hexyl. In some embodiments, R³ is C₄-C₆ alkyl. In some embodiments, R³ is C₃-C₆ alkenyl. In some embodiments, R³ is C₄-C₆ alkenyl. In some embodiments, R³ is C₃-C₆ alkynyl. In some embodiments, R³ is C₄-C₆ alkynyl. In some embodiments, R³ is C₃-C₁₂ cycloalkyl, such as cyclopropyl, cyclobutyl, cyclopentyl, or cyclohexyl. In some embodiments, R³ is C₃-C₆ cycloalkyl. In some embodiments, R³ is C₃-C₆ cycloalkylalkyl, such as —(CH₂)₁₋₃(C₃-C₆ cycloalkyl). In some embodiments, R³ is C₆-C₁₀ arylalkyl, such as benzyl. In some embodiments, R³ is 5- to 10-membered heteroarylalkyl, such as —(CH₂)₁₋₃(5- to 10-membered heteroaryl) or —(CH₂)₁₋₃(5- to 6-membered heteroaryl). In some embodiments, the 5- to 10-membered heteroarylalkyl contains 1-2 nitrogen atoms. In some embodiments, R³ is 5- to 10-membered heterocyclylalkyl, such as —(CH₂)₁₋₃(5- to 10-membered heterocyclyl) or —(CH₂)₁₋₂(5 to 6-membered heterocyclyl). In some embodiments, the 5- to 10-membered heterocyclylalkyl contains 1-2 nitrogen atoms.

In some embodiments, R³ is C₃-C₆ alkyl optionally substituted by 1-3 substituents selected from halo and —C(═O)NH₂, C₂-C₆ alkenyl, or C₃-C₆ cycloalkylalkyl. In some embodiments, R³ is C₂-C₆ alkyl optionally substituted by 1-3 substituents selected from halo, C₁-C₃ alkoxy, hydroxy, —NH₂, —SO₂(C₁-C₃ alkyl), and —C(═O)NH₂; C₂-C₆ alkenyl; C₃-C₆ cycloalkylalkyl; 5- to 6-membered heteroarylalkyl; 5- to 6-membered heterocyclylalkyl; or C₆ arylalkyl. In some embodiments, R³ is C₂ alkyl substituted by 1-3 substituents selected from C₁-C₃ alkoxy, hydroxy, —NH₂, and —SO₂(C₁-C₃ alkyl). In some embodiments, R³ is:

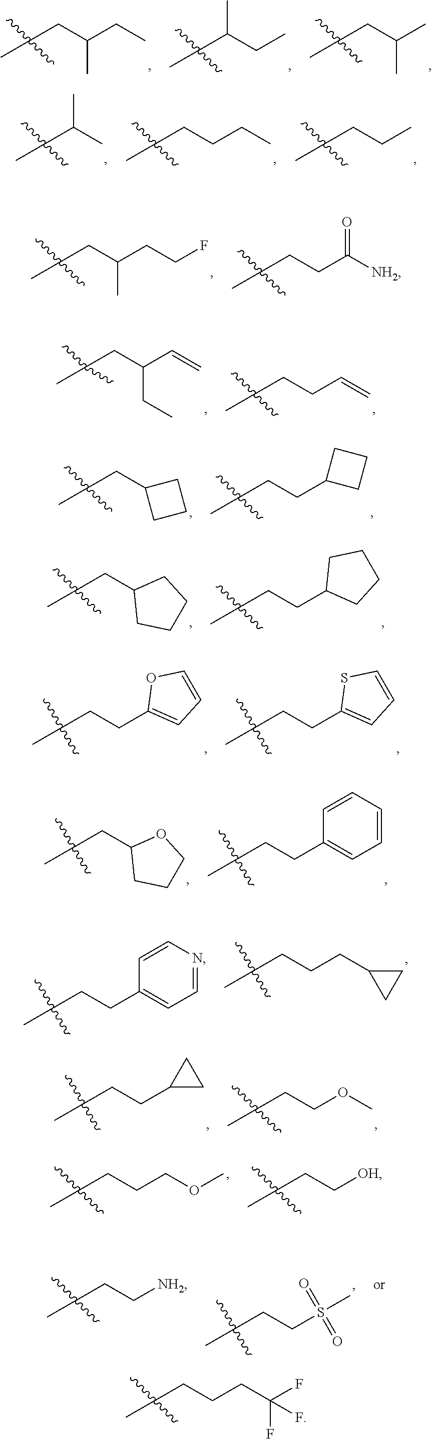

In some embodiments, R³ is:

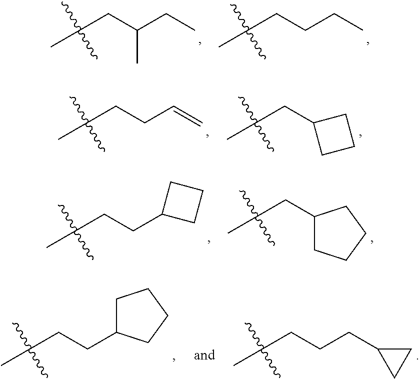

In some embodiments, R³ is 2-methylbutyl.

In some embodiments, R⁴ is C₆-C₁₀ aryl, 5- to 10-membered heteroaryl, or 5- to 10-membered heterocyclyl, wherein the 5- to 10-membered heteroaryl or 5- to 10-membered heterocyclyl contains 1-3 heteroatoms selected from nitrogen and oxygen. In some embodiments, R⁴ is C₆-C₁₀ aryl, such as phenyl. In some embodiments, R⁴ is 5- to 10-membered heteroaryl containing 1-2 nitrogen atoms. In some embodiments, R⁴ is 5- to 10-membered heterocyclyl. In some embodiments, R⁴ is 5- to 9-membered heterocyclyl containing 1-2 nitrogen atoms. In some embodiments, R⁴ is 5- to 9-membered heterocyclyl containing 1-2 oxygen atoms. In some embodiments, R⁴ is 5- to 9-membered heterocyclyl containing 1 nitrogen atom and 1 oxygen atom.

In some embodiments, R⁴ is C₆-C₁₀ aryl optionally substituted with 1-3 substituents selected from halo, hydroxyl, C₁-C₆ haloalkyl, and C₁-C₆ haloalkoxy. In some embodiments, R⁴ is phenyl substituted with 1-3 substituents selected from —CF₃, —OCHF₂, —OH, fluoro, and chloro. In some embodiments, R⁴ is:

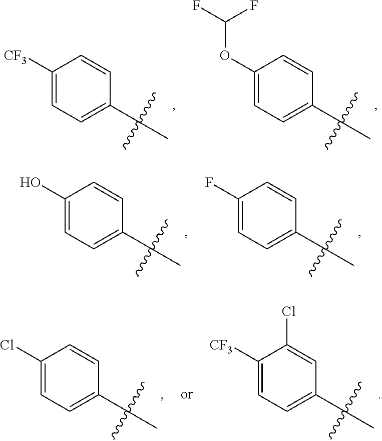 [SMILES: *c1ccc(C(F)(F)F)cc1 ; *c1ccc(OC(F)F)cc1 ; *c1ccc(O)cc1 ; *c1ccc(F)cc1 ; *c1ccc(Cl)cc1 ; *c1ccc(C(F)(F)F)c(Cl)c1]

In some embodiments, R⁴ is:

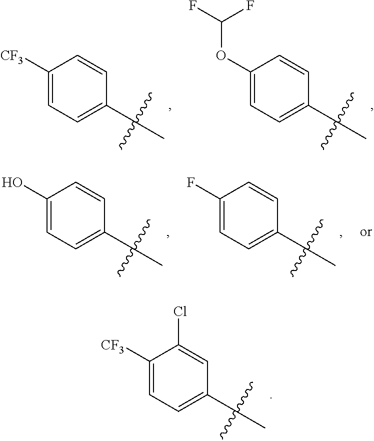 [SMILES: *c1ccc(C(F)(F)F)cc1 ; *c1ccc(OC(F)F)cc1 ; *c1ccc(O)cc1 ; *c1ccc(F)cc1 ; *c1ccc(C(F)(F)F)c(Cl)c1]

In some embodiments, R⁴ is 5- to 10-membered heteroaryl optionally substituted with 1-3 substituents selected from halo, hydroxyl, C₁-C₆ haloalkyl, and C₁-C₆ haloalkoxy. In some embodiments, R⁴ is pyridyl or indolyl optionally substituted with 1-3 substituents selected from halo, hydroxyl, C₁-C₆ haloalkyl, and C₁-C₆ haloalkoxy. In some embodiments, R⁴ is

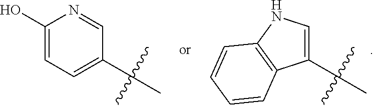 [SMILES: *c1ccc(O)nc1 ; *c1c[nH]c2ccccc12]

In some embodiments, R⁴ is pyridyl substituted with 1-3 substituents selected from halo, hydroxyl, C₁-C₆ haloalkyl, and C₁-C₆ haloalkoxy. In some embodiments, R⁴ is

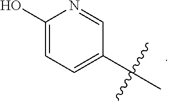 [SMILES: *c1ccc(O)nc1]

In some embodiments, R⁴ is 5- to 10-membered heterocyclyl optionally substituted with 1-3 substituents selected from halo, hydroxyl, C₁-C₆ haloalkyl, and C₁-C₆ haloalkoxy. In some embodiments, R⁴ is indolinyl.

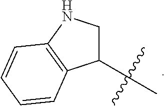 [SMILES: *C1CNc2ccccc21]

In some embodiments, -L-R⁴ is —CH₂(phenyl) or —C(O)(phenyl), wherein the phenyl is substituted by 1-3 substituents selected from C₁-C₃ haloalkyl, C₁-C₃ haloalkoxy, halo, and hydroxy. In some embodiments, -L-R⁴ is —CH₂(pyridyl) or —C(O)(pyridyl), wherein the pyridyl is substituted by 1-3 substituents selected from C₁-C₃ haloalkyl, C₁-C₃ haloalkoxy, halo, and hydroxy. In some embodiments, -L-R⁴ is:

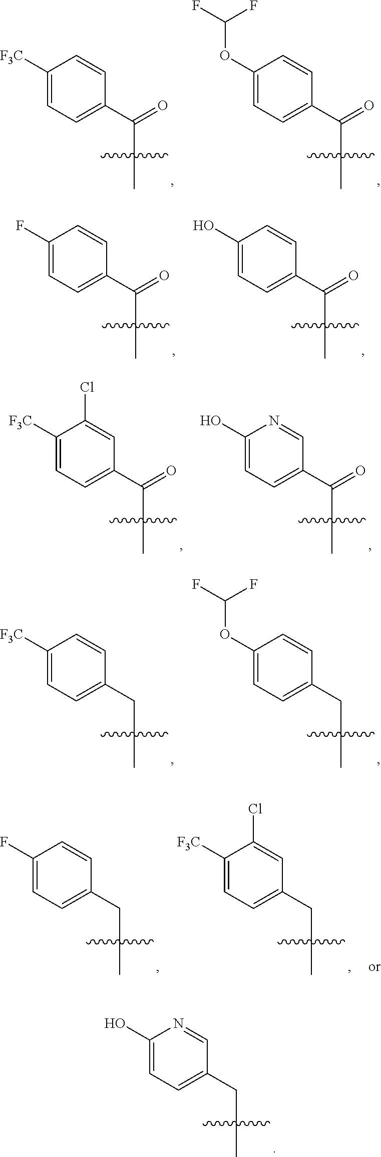

In some embodiments, each R⁵ is independently C₁-C₆ alkyl, oxo, or halo. In some embodiments, R⁵ is C₁-C₆ alkyl, such as methyl, ethyl, or propyl. In some embodiments, R⁵ is oxo. In some embodiments, R⁵ is halo, such as fluoro, chloro, or bromo. In some embodiments, R⁵ is oxo or halo. In some embodiments, R⁵ is oxo or fluoro.

In some embodiments, R⁶ is H, C₁-C₆ alkyl, or oxo. In some embodiments, R⁶ is H. In some embodiments, R⁶ is C₁-C₆ alkyl, such as methyl, ethyl, or propyl. In some embodiments, R⁶ is oxo.

In some embodiments, R⁷ is H or oxo. In some embodiments, R⁷ is H. In some embodiments, R⁷ is oxo.

In some embodiments, m is 1. In other embodiments, m is 2.

In some embodiments, n is 0. In other embodiments, n is an integer from 1 to 3. In some embodiments, n is 1. In some embodiments, n is 2. In some embodiments, n is 3.

In any embodiments of Formula (I), or variations thereof, each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to three substituents selected from hydroxyl, halo (such as fluoro, chloro, or bromo), amino, C₁-C₆ haloalkyl (such as —CF₃ or —CHF₂), C₁-C₆ alkoxy (such as methoxy or ethoxy), C₁-C₆ haloalkoxy (such as —OCHF₂ or —OCF₃), and —(C═O)NH₂.

In some embodiments, the compound of Formula (I) is a compound of Formula (II), (IIa), (IIb), (IIc), (IId), or (IIe).

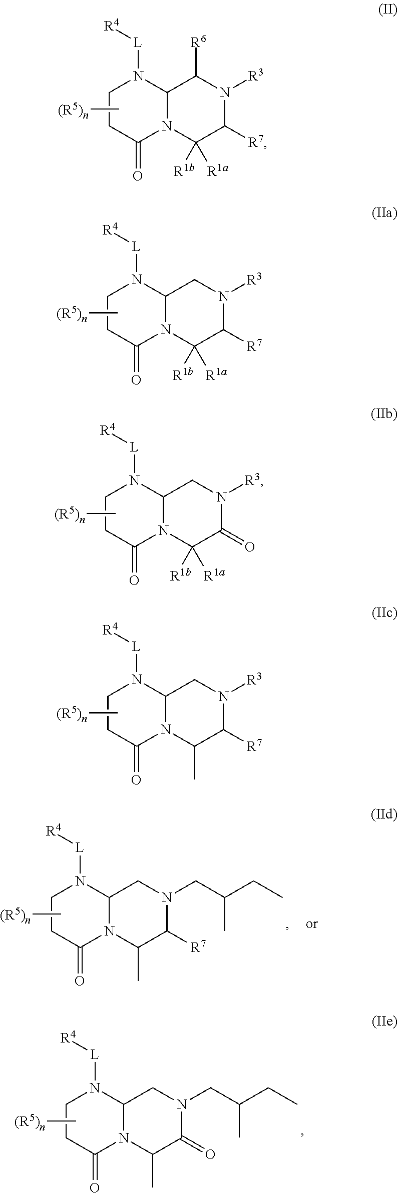

or a pharmaceutically acceptable salt thereof, wherein L, R^{1a}, R^{1b}, R³, R⁴, R⁵, R⁶, R⁷, and n are as described for Formula (I). In some embodiments, the compound is of Formula (II) or a pharmaceutically acceptable salt thereof. In some embodiments, the compound is of Formula (IIa) or a pharmaceutically acceptable salt thereof. In some embodiments, the compound is of Formula (IIb) or a pharmaceutically acceptable salt thereof. In some embodiments, the compound is of Formula (IIc) or a pharmaceutically acceptable salt thereof. In some embodiments, the compound is of Formula (IId) or a pharmaceutically acceptable salt thereof. In some embodiments, the compound is of Formula (IIe) or a pharmaceutically acceptable salt thereof.

In some embodiments, the compound of Formula (I) is a compound of Formula (IIIa), (IIb), (IIc), or (IIId):

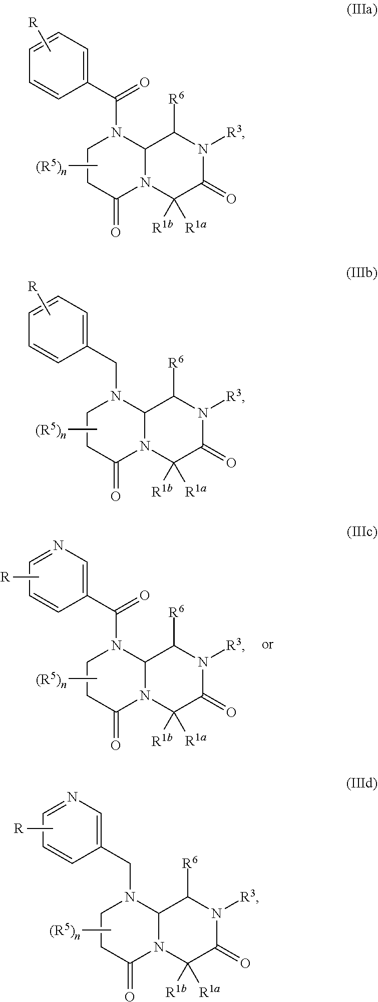

or a pharmaceutically acceptable salt thereof, wherein R^{1a}, R^{1b}, R³, R⁵, R⁶, and n are as described for Formula (I), and R represents one or more optional substituents, such as hydroxyl, halo, amino, C₁-C₆ haloalkyl, C₁-C₆ haloalkoxy, as described for Formula (I). In some embodiments, the compound is of Formula (IIIa) or a pharmaceutically acceptable salt thereof. In some embodiments, the compound is of Formula (IIIb) or a pharmaceutically acceptable salt thereof. In some embodiments, the compound is of Formula (IIIc) or a pharmaceutically acceptable salt thereof. In some embodiments, the compound is of Formula (IIId) or a pharmaceutically acceptable salt thereof.

In some embodiments, the compound of Formula (I) is a compound of Formula (IVa), (IVb), (IVc), or (IVd):

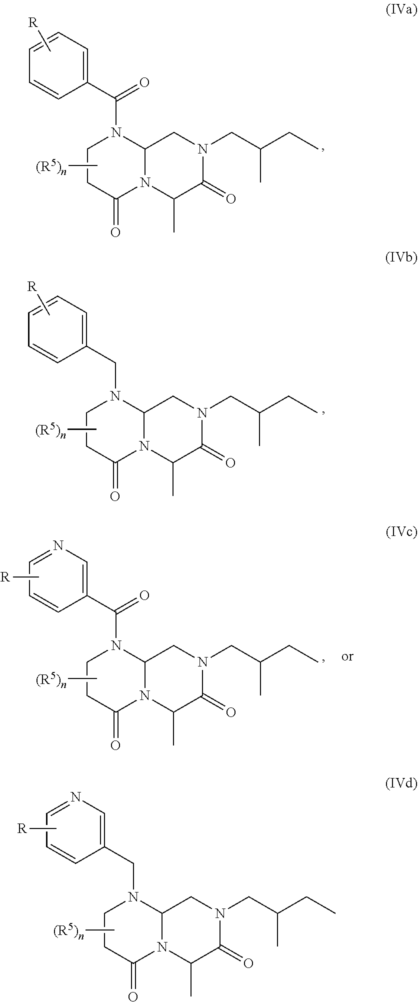

or a pharmaceutically acceptable salt thereof, wherein R⁵ and n are as described for Formula (I), and R represents one or more optional substituents, such as hydroxyl, halo, amino, C₁-C₆ haloalkyl, C₁-C₆ haloalkoxy, as described for Formula (I). In some embodiments, the compound is of Formula (IVa) or a pharmaceutically acceptable salt thereof. In some embodiments, the compound is of Formula (IVb) or a pharmaceutically acceptable salt thereof. In some embodiments, the compound is of Formula (IVc) or a pharmaceutically acceptable salt thereof. In some embodiments, the compound is of Formula (IVd) or a pharmaceutically acceptable salt thereof.

In some embodiments, the compound of Formula (I) is a compound of Formula (V):

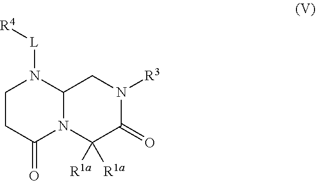

or a pharmaceutically acceptable salt thereof, wherein L, R^{1a}, R^{1b}, R³, and R⁴ are as described for Formula (I). In some embodiments, L is —C(═O)— or —CH₂—; R^{1a} and R^{1b} are independently H or C₁-C₃ alkyl optionally substituted with —CO₂H; R³ is C₄-C₅ alkyl, C₄-C₅ alkenyl, or C₁-C₃ alkyl substituted with C₃-C₅ cycloalkyl; and R⁴ is phenyl or pyridyl substituted with 1-3 substituents selected from —CF₃, —OCHF₂, —OH, fluoro, and chloro. In some variations, one of R^{1a} and R^{1b} is H and the other is C₁-C₃ alkyl, such as methyl.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to L of Formula (I) may be combined with every description, variation, embodiment, or aspect of R^{1a}, R^{1b}, R², R³, R⁴, R⁵, R⁶, R⁷, and n the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (IIa), (IIb), (IIc), (IId), (IIe), (IIIa), (IIIb), (IIIc), (IIId), (IVa), (IVb), (IVc), (IVd), and (V), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

### Table 1.

TABLE 1

| Compound | Structure |
|---|---|
| 1a | 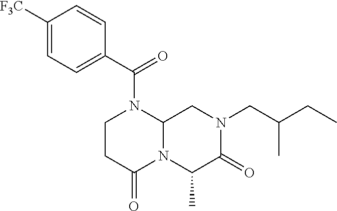 [SMILES: CCC(C)CN1CC2N(C(=O)c3ccc(C(F)(F)F)cc3)CCC(=O)N2[C@@H](C)C1=O] |
| 1b | 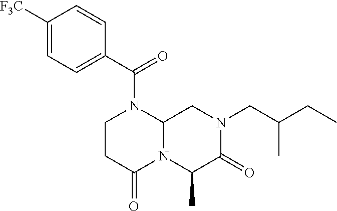 [SMILES: CCC(C)CN1CC2N(C(=O)c3ccc(C(F)(F)F)cc3)CCC(=O)N2[C@H](C)C1=O] |
| 2a | 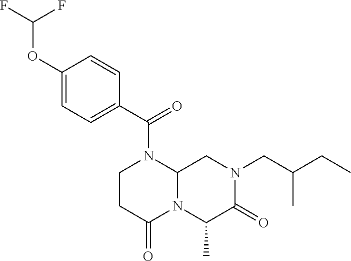 [SMILES: CCC(C)CN1CC2N(C(=O)c3ccc(OC(F)F)cc3)CCC(=O)N2[C@@H](C)C1=O] |
| 2b | 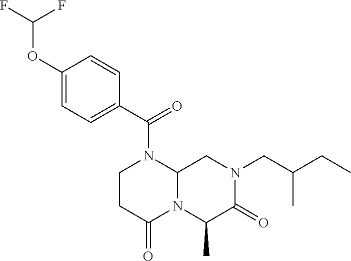 [SMILES: CCC(C)CN1CC2N(C(=O)c3ccc(OC(F)F)cc3)CCC(=O)N2[C@H](C)C1=O] |
| 3a | 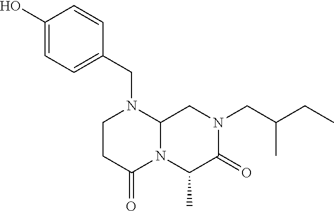 [SMILES: CCC(C)CN1CC2N(Cc3ccc(O)cc3)CCC(=O)N2[C@@H](C)C1=O] |
| 3b | 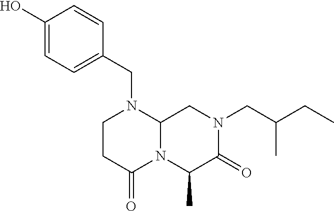 [SMILES: CCC(C)CN1CC2N(Cc3ccc(O)cc3)CCC(=O)N2[C@H](C)C1=O] |
| 4a | 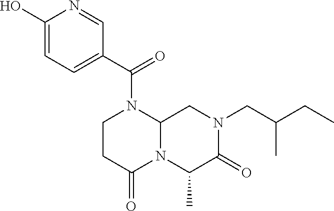 [SMILES: CCC(C)CN1CC2N(C(=O)c3ccc(O)nc3)CCC(=O)N2[C@@H](C)C1=O] |
| 4b | 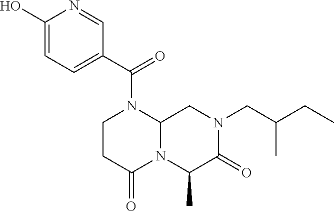 [SMILES: CCC(C)CN1CC2N(C(=O)c3ccc(O)nc3)CCC(=O)N2[C@H](C)C1=O] |
| 5a | 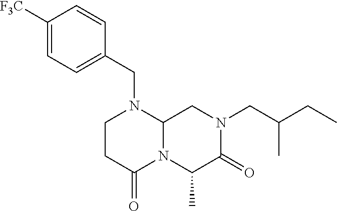 [SMILES: CCC(C)CN1CC2N(Cc3ccc(C(F)(F)F)cc3)CCC(=O)N2[C@@H](C)C1=O] |
| 5b | 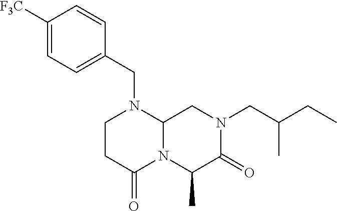 [SMILES: CCC(C)CN1CC2N(Cc3ccc(C(F)(F)F)cc3)CCC(=O)N2[C@H](C)C1=O] |
| 6a | 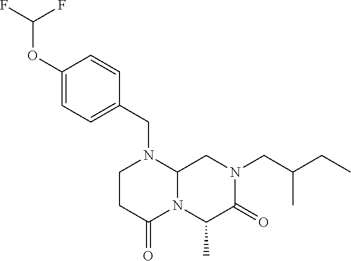 [SMILES: CCC(C)CN1CC2N(Cc3ccc(OC(F)F)cc3)CCC(=O)N2[C@@H](C)C1=O] |
| 6b | 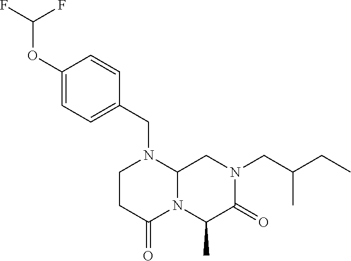 [SMILES: CCC(C)CN1CC2N(Cc3ccc(OC(F)F)cc3)CCC(=O)N2[C@H](C)C1=O] |
| 7a | 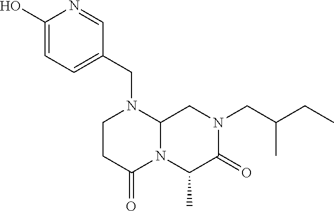 [SMILES: CCC(C)CN1CC2N(Cc3ccc(O)nc3)CCC(=O)N2[C@@H](C)C1=O] |
| 7b | 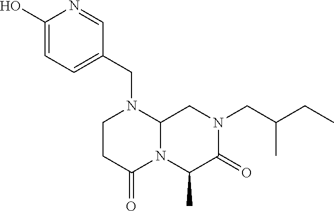 [SMILES: CCC(C)CN1CC2N(Cc3ccc(O)nc3)CCC(=O)N2[C@H](C)C1=O] |
| 8a | 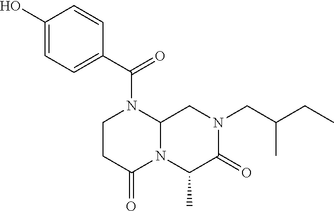 [SMILES: CCC(C)CN1CC2N(C(=O)c3ccc(O)cc3)CCC(=O)N2[C@@H](C)C1=O] |
| 8b | 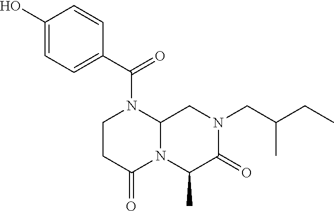 [SMILES: CCC(C)CN1CC2N(C(=O)c3ccc(O)cc3)CCC(=O)N2[C@H](C)C1=O] |
| 9 | 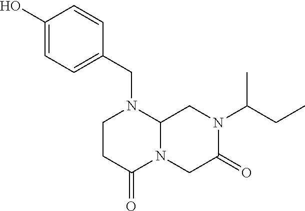 [SMILES: CCC(C)N1CC2N(Cc3ccc(O)cc3)CCC(=O)N2CC1=O] |
| 10 | 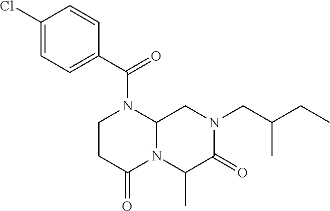 [SMILES: CCC(C)CN1CC2N(C(=O)c3ccc(Cl)cc3)CCC(=O)N2C(C)C1=O] |
| 11 | 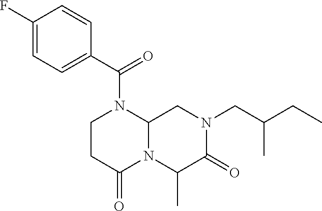 [SMILES: CCC(C)CN1CC2N(C(=O)c3ccc(F)cc3)CCC(=O)N2C(C)C1=O] |
| 12 | 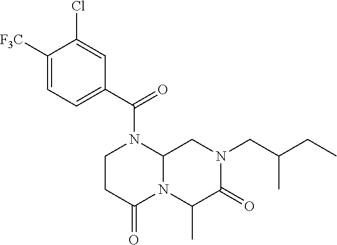 [SMILES: CCC(C)CN1CC2N(C(=O)c3ccc(C(F)(F)F)c(Cl)c3)CCC(=O)N2C(C)C1=O] |
| 13 | 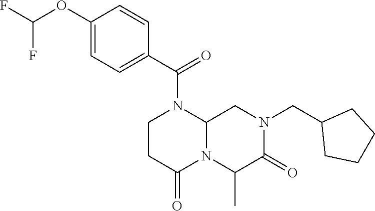 [SMILES: CC1C(=O)N(CC2CCCC2)CC2N(C(=O)c3ccc(OC(F)F)cc3)CCC(=O)N12] |
| 14 | 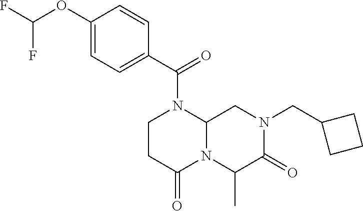 [SMILES: CC1C(=O)N(CC2CCC2)CC2N(C(=O)c3ccc(OC(F)F)cc3)CCC(=O)N12] |
| 15 | 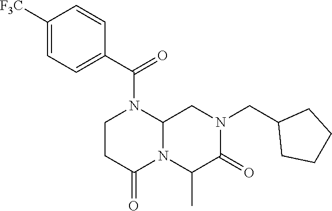 [SMILES: CC1C(=O)N(CC2CCCC2)CC2N(C(=O)c3ccc(C(F)(F)F)cc3)CCC(=O)N12] |
| 16 | 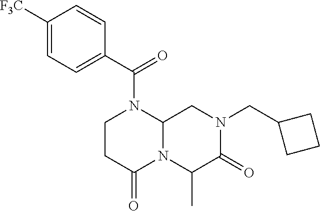 [SMILES: CC1C(=O)N(CC2CCC2)CC2N(C(=O)c3ccc(C(F)(F)F)cc3)CCC(=O)N12] |
| 17 | 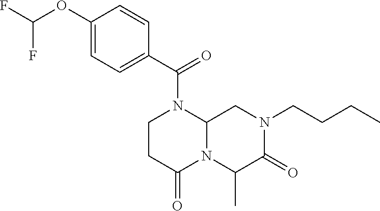 [SMILES: CCCCN1CC2N(C(=O)c3ccc(OC(F)F)cc3)CCC(=O)N2C(C)C1=O] |
| 18 | 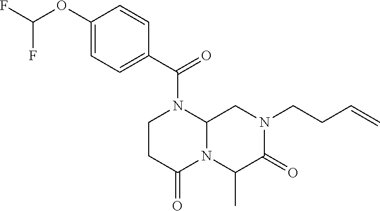 [SMILES: C=CCCN1CC2N(C(=O)c3ccc(OC(F)F)cc3)CCC(=O)N2C(C)C1=O] |
| 19 | 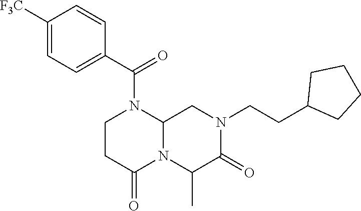 [SMILES: CC1C(=O)N(CCC2CCCC2)CC2N(C(=O)c3ccc(C(F)(F)F)cc3)CCC(=O)N12] |
| 20 | 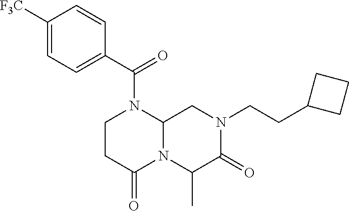 [SMILES: CC1C(=O)N(CCC2CCC2)CC2N(C(=O)c3ccc(C(F)(F)F)cc3)CCC(=O)N12] |
| 21 | 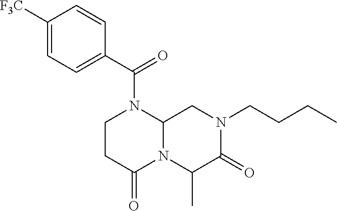 [SMILES: CCCCN1CC2N(C(=O)c3ccc(C(F)(F)F)cc3)CCC(=O)N2C(C)C1=O] |
| 22 | 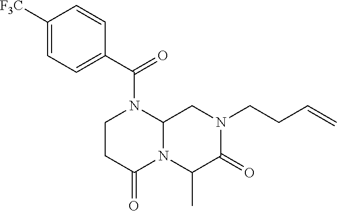 [SMILES: C=CCCN1CC2N(C(=O)c3ccc(C(F)(F)F)cc3)CCC(=O)N2C(C)C1=O] |
| 23 | 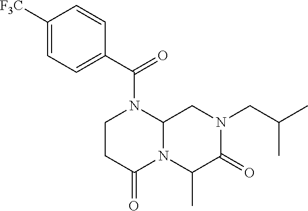 [SMILES: CC(C)CN1CC2N(C(=O)c3ccc(C(F)(F)F)cc3)CCC(=O)N2C(C)C1=O] |
| 24 | 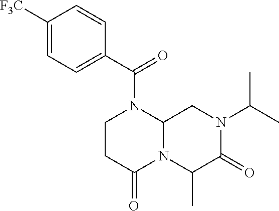 [SMILES: CC(C)N1CC2N(C(=O)c3ccc(C(F)(F)F)cc3)CCC(=O)N2C(C)C1=O] |
| 25 | 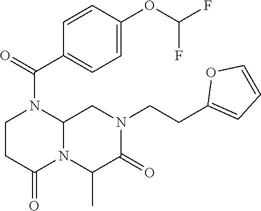 [SMILES: CC1C(=O)N(CCc2ccco2)CC2N(C(=O)c3ccc(OC(F)F)cc3)CCC(=O)N12] |
| 26 | 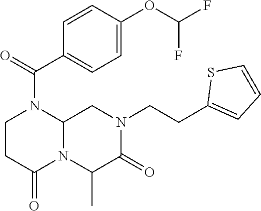 [SMILES: CC1C(=O)N(CCc2cccs2)CC2N(C(=O)c3ccc(OC(F)F)cc3)CCC(=O)N12] |
| 27 | 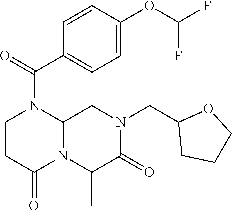 [SMILES: CC1C(=O)N(CC2CCCO2)CC2N(C(=O)c3ccc(OC(F)F)cc3)CCC(=O)N12] |
| 28 | 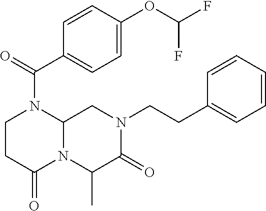 [SMILES: CC1C(=O)N(CCc2ccccc2)CC2N(C(=O)c3ccc(OC(F)F)cc3)CCC(=O)N12] |
| 29 | 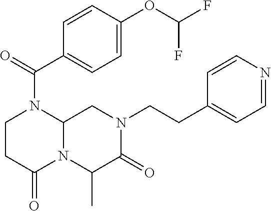 [SMILES: CC1C(=O)N(CCc2ccncc2)CC2N(C(=O)c3ccc(OC(F)F)cc3)CCC(=O)N12] |
| 30 | 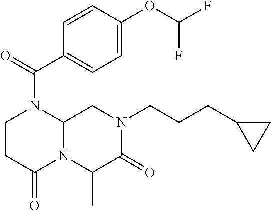 [SMILES: CC1C(=O)N(CCCC2CC2)CC2N(C(=O)c3ccc(OC(F)F)cc3)CCC(=O)N12] |
| 31 | 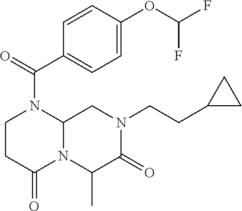 [SMILES: CC1C(=O)N(CCC2CC2)CC2N(C(=O)c3ccc(OC(F)F)cc3)CCC(=O)N12] |
| 32 | 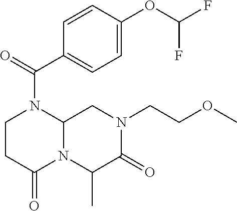 [SMILES: COCCN1CC2N(C(=O)c3ccc(OC(F)F)cc3)CCC(=O)N2C(C)C1=O] |
| 33 | 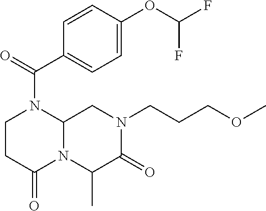 [SMILES: COCCCN1CC2N(C(=O)c3ccc(OC(F)F)cc3)CCC(=O)N2C(C)C1=O] |
| 34 | 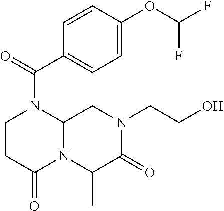 [SMILES: CC1C(=O)N(CCO)CC2N(C(=O)c3ccc(OC(F)F)cc3)CCC(=O)N12] |
| 35 | 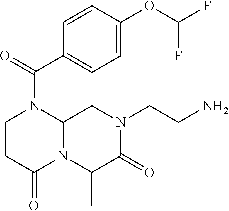 [SMILES: CC1C(=O)N(CCN)CC2N(C(=O)c3ccc(OC(F)F)cc3)CCC(=O)N12] |
| 36 | 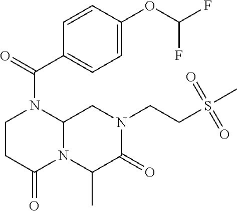 [SMILES: CC1C(=O)N(CCS(C)(=O)=O)CC2N(C(=O)c3ccc(OC(F)F)cc3)CCC(=O)N12] |
| 37 | 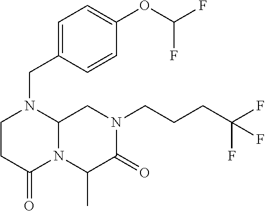 [SMILES: CC1C(=O)N(CCCC(F)(F)F)CC2N(Cc3ccc(OC(F)F)cc3)CCC(=O)N12] |
| 38 | 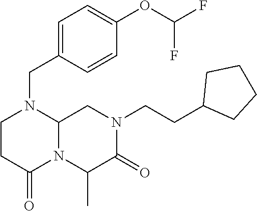 [SMILES: CC1C(=O)N(CCC2CCCC2)CC2N(Cc3ccc(OC(F)F)cc3)CCC(=O)N12] |
| 39 | 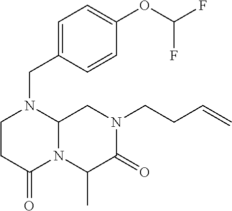 [SMILES: C=CCCN1CC2N(Cc3ccc(OC(F)F)cc3)CCC(=O)N2C(C)C1=O] |
| 40 | 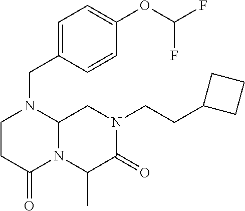 [SMILES: CC1C(=O)N(CCC2CCC2)CC2N(Cc3ccc(OC(F)F)cc3)CCC(=O)N12] |
| 41 | 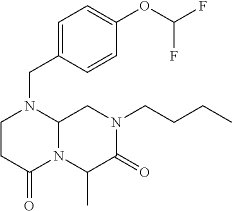 [SMILES: CCCCN1CC2N(Cc3ccc(OC(F)F)cc3)CCC(=O)N2C(C)C1=O] |
| 42 | 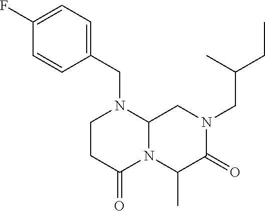 [SMILES: CCC(C)CN1CC2N(Cc3ccc(F)cc3)CCC(=O)N2C(C)C1=O] |
| 43 | 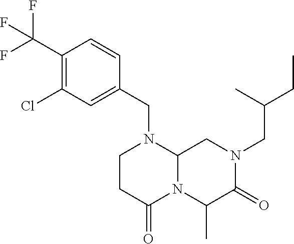 [SMILES: CCC(C)CN1CC2N(Cc3ccc(C(F)(F)F)c(Cl)c3)CCC(=O)N2C(C)C1=O] |
| 44 | 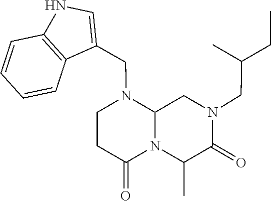 [SMILES: CCC(C)CN1CC2N(Cc3c[nH]c4ccccc34)CCC(=O)N2C(C)C1=O] |
| 45 | 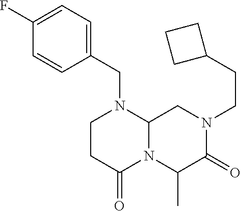 [SMILES: CC1C(=O)N(CCC2CCC2)CC2N(Cc3ccc(F)cc3)CCC(=O)N12] |
| 46 | 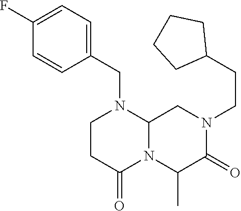 [SMILES: CC1C(=O)N(CCC2CCCC2)CC2N(Cc3ccc(F)cc3)CCC(=O)N12] |
| 47 | 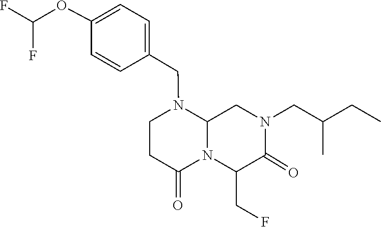 |
| 48 | 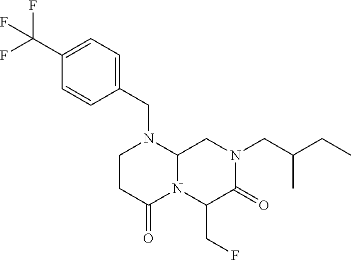 [SMILES: CCC(C)CN1CC2N(Cc3ccc(C(F)(F)F)cc3)CCC(=O)N2C(CF)C1=O] |
| 49 | 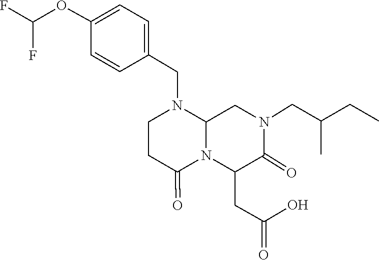 [SMILES: CCC(C)CN1CC2N(Cc3ccc(OC(F)F)cc3)CCC(=O)N2C(CC(=O)O)C1=O] |
| 50 | 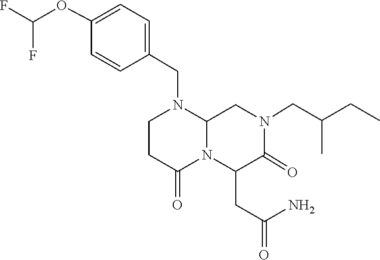 [SMILES: CCC(C)CN1CC2N(Cc3ccc(OC(F)F)cc3)CCC(=O)N2C(CC(N)=O)C1=O] |
| 51 | 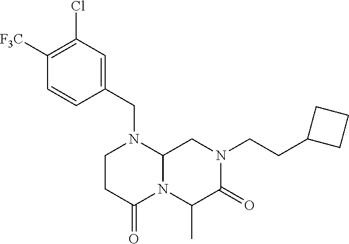 [SMILES: CC1C(=O)N(CCC2CCC2)CC2N(Cc3ccc(C(F)(F)F)c(Cl)c3)CCC(=O)N12] |
| 52 | 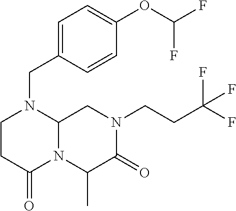 [SMILES: CC1C(=O)N(CCC(F)(F)F)CC2N(Cc3ccc(OC(F)F)cc3)CCC(=O)N12] |
| 53 | 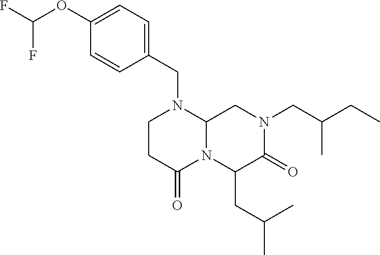 [SMILES: CCC(C)CN1CC2N(Cc3ccc(OC(F)F)cc3)CCC(=O)N2C(CC(C)C)C1=O] |
| 54 | 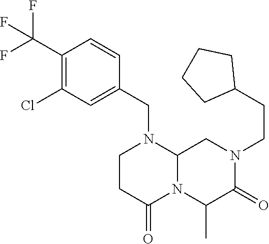 [SMILES: CC1C(=O)N(CCC2CCCC2)CC2N(Cc3ccc(C(F)(F)F)c(Cl)c3)CCC(=O)N12] |
| 55 | 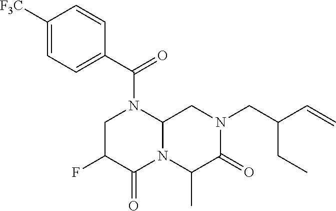 [SMILES: C=CC(CC)CN1CC2N(C(=O)c3ccc(C(F)(F)F)cc3)CC(F)C(=O)N2C(C)C1=O] |
| 56 | 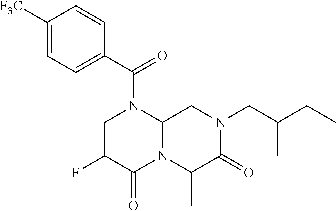 [SMILES: CCC(C)CN1CC2N(C(=O)c3ccc(C(F)(F)F)cc3)CC(F)C(=O)N2C(C)C1=O] |
| 57 | 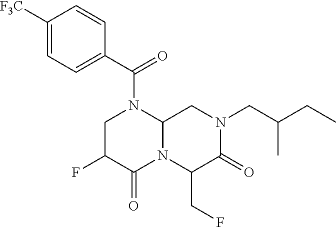 [SMILES: CCC(C)CN1CC2N(C(=O)c3ccc(C(F)(F)F)cc3)CC(F)C(=O)N2C(CF)C1=O] |
| 58 | 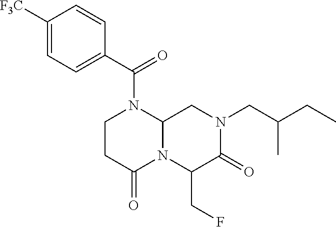 [SMILES: CCC(C)CN1CC2N(C(=O)c3ccc(C(F)(F)F)cc3)CCC(=O)N2C(CF)C1=O] |
| 59 | 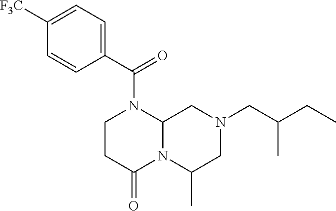 [SMILES: CCC(C)CN1CC(C)N2C(=O)CCN(C(=O)c3ccc(C(F)(F)F)cc3)C2C1] |
| 60 | 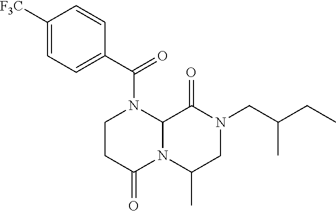 [SMILES: CCC(C)CN1CC(C)N2C(=O)CCN(C(=O)c3ccc(C(F)(F)F)cc3)C2C1=O] |
| 61 | 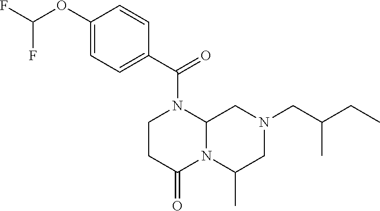 [SMILES: CCC(C)CN1CC(C)N2C(=O)CCN(C(=O)c3ccc(OC(F)F)cc3)C2C1] |
| 62 | 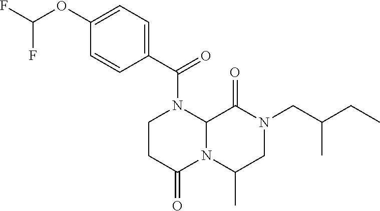 [SMILES: CCC(C)CN1CC(C)N2C(=O)CCN(C(=O)c3ccc(OC(F)F)cc3)C2C1=O] |
| 63 | 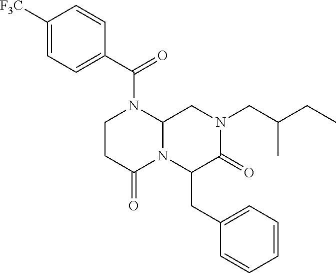 [SMILES: CCC(C)CN1CC2N(C(=O)c3ccc(C(F)(F)F)cc3)CCC(=O)N2C(Cc2ccccc2)C1=O] |
| 64 | 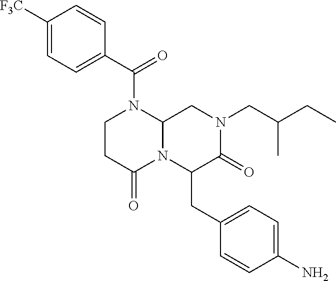 [SMILES: CCC(C)CN1CC2N(C(=O)c3ccc(C(F)(F)F)cc3)CCC(=O)N2C(Cc2ccc(N)cc2)C1=O] |
| 65 | 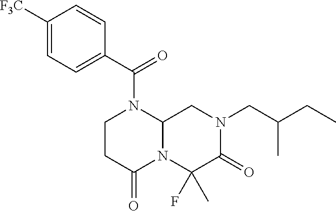 [SMILES: CCC(C)CN1CC2N(C(=O)c3ccc(C(F)(F)F)cc3)CCC(=O)N2C(C)(F)C1=O] |
| 66 | 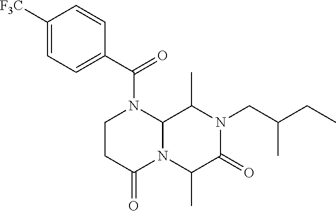 [SMILES: CCC(C)CN1C(=O)C(C)N2C(=O)CCN(C(=O)c3ccc(C(F)(F)F)cc3)C2C1C] |
| 67 | 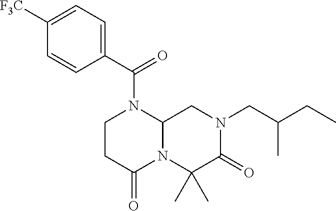 [SMILES: CCC(C)CN1CC2N(C(=O)c3ccc(C(F)(F)F)cc3)CCC(=O)N2C(C)(C)C1=O] |
| 68 | 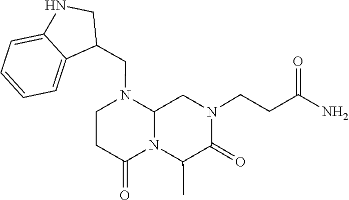 [SMILES: CC1C(=O)N(CCC(N)=O)CC2N(CC3CNc4ccccc43)CCC(=O)N12] |
| 69 | 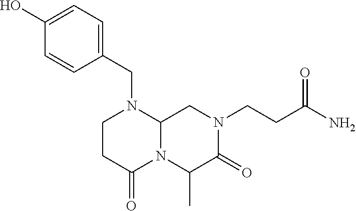 [SMILES: CC1C(=O)N(CCC(N)=O)CC2N(Cc3ccc(O)cc3)CCC(=O)N12] |
| 70 | 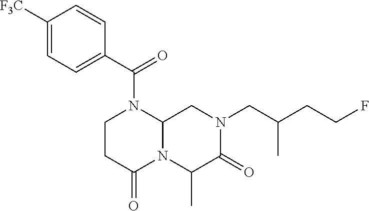 [SMILES: CC(CCF)CN1CC2N(C(=O)c3ccc(C(F)(F)F)cc3)CCC(=O)N2C(C)C1=O] |
| 71 | 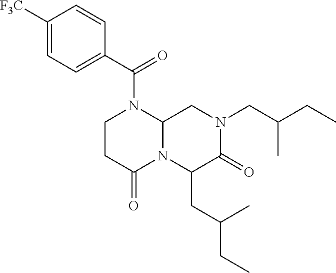 [SMILES: CCC(C)CC1C(=O)N(CC(C)CC)CC2N(C(=O)c3ccc(C(F)(F)F)cc3)CCC(=O)N12] |
| 72 | 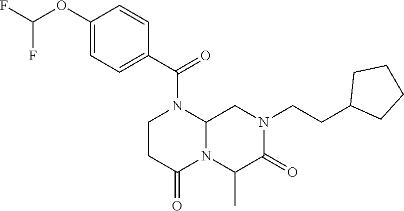 [SMILES: CC1C(=O)N(CCC2CCCC2)CC2N(C(=O)c3ccc(OC(F)F)cc3)CCC(=O)N12] |
| 73 | 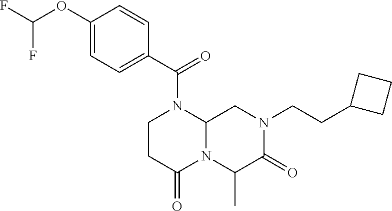 [SMILES: CC1C(=O)N(CCC2CCC2)CC2N(C(=O)c3ccc(OC(F)F)cc3)CCC(=O)N12] |
| 74 | 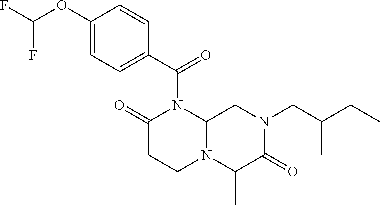 [SMILES: CCC(C)CN1CC2N(C(=O)c3ccc(OC(F)F)cc3)C(=O)CCN2C(C)C1=O] |

or a pharmaceutically acceptable salt thereof.

In some embodiments, the compound of Formula (I) is not Compound 3a, 3b, 9, 10, 13, 15, 16, 18, 21, 23-29, 31-41, 43-48, 50, 52, or 54.

In some embodiments, provided is a compound selected from the compounds in Table 1A or a pharmaceutically acceptable salt thereof. Although certain compounds described in the present disclosure, including in Table 1A, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1A, are herein described.

### Table 1A.

TABLE 1A

| Compound | Structure |
|---|---|
| 1a | 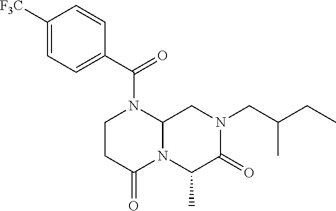 [SMILES: CCC(C)CN1CC2N(C(=O)c3ccc(C(F)(F)F)cc3)CCC(=O)N2[C@@H](C)C1=O] |
| 1b | 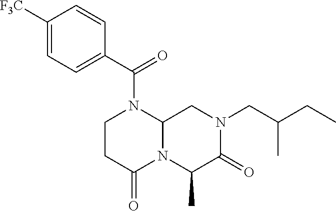 [SMILES: CCC(C)CN1CC2N(C(=O)c3ccc(C(F)(F)F)cc3)CCC(=O)N2[C@H](C)C1=O] |
| 2a | 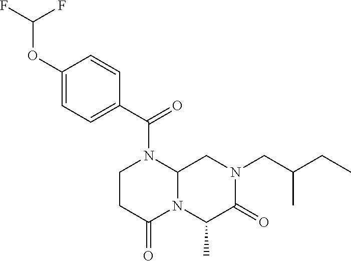 [SMILES: CCC(C)CN1CC2N(C(=O)c3ccc(OC(F)F)cc3)CCC(=O)N2[C@@H](C)C1=O] |
| 2b | 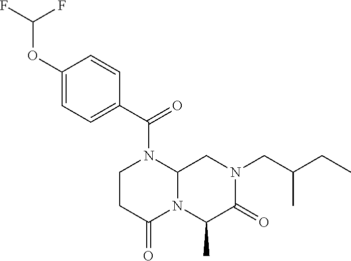 [SMILES: CCC(C)CN1CC2N(C(=O)c3ccc(OC(F)F)cc3)CCC(=O)N2[C@H](C)C1=O] |
| 4a | 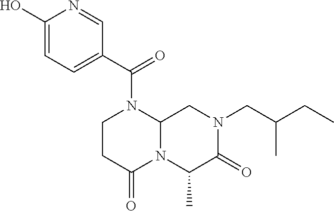 [SMILES: CCC(C)CN1CC2N(C(=O)c3ccc(O)nc3)CCC(=O)N2[C@@H](C)C1=O] |
| 4b | 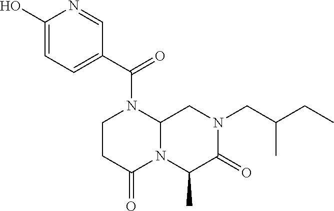 [SMILES: CCC(C)CN1CC2N(C(=O)c3ccc(O)nc3)CCC(=O)N2[C@H](C)C1=O] |
| 5a | 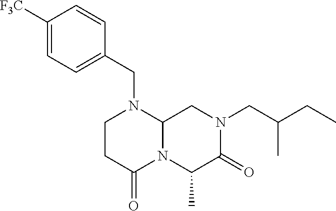 [SMILES: CCC(C)CN1CC2N(Cc3ccc(C(F)(F)F)cc3)CCC(=O)N2[C@@H](C)C1=O] |
| 5b | 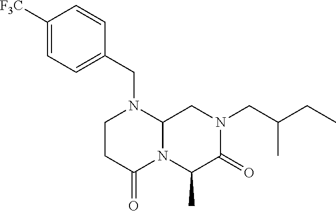 [SMILES: CCC(C)CN1CC2N(Cc3ccc(C(F)(F)F)cc3)CCC(=O)N2[C@H](C)C1=O] |
| 6a | 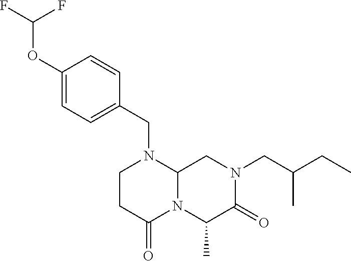 [SMILES: CCC(C)CN1CC2N(Cc3ccc(OC(F)F)cc3)CCC(=O)N2[C@@H](C)C1=O] |
| 6b | 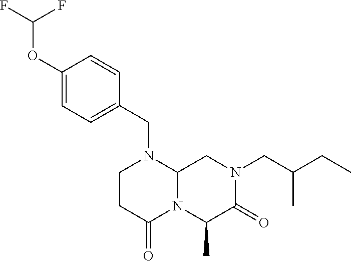 [SMILES: CCC(C)CN1CC2N(Cc3ccc(OC(F)F)cc3)CCC(=O)N2[C@H](C)C1=O] |
| 7a | 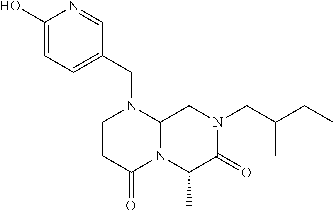 [SMILES: CCC(C)CN1CC2N(Cc3ccc(O)nc3)CCC(=O)N2[C@@H](C)C1=O] |
| 7b | 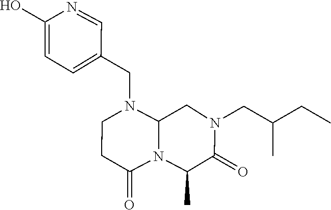 [SMILES: CCC(C)CN1CC2N(Cc3ccc(O)nc3)CCC(=O)N2[C@H](C)C1=O] |
| 8a | 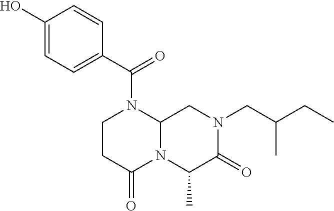 [SMILES: CCC(C)CN1CC2N(C(=O)c3ccc(O)cc3)CCC(=O)N2[C@@H](C)C1=O] |
| 8b | 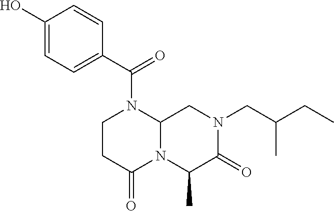 [SMILES: CCC(C)CN1CC2N(C(=O)c3ccc(O)cc3)CCC(=O)N2[C@H](C)C1=O] |
| 11 | 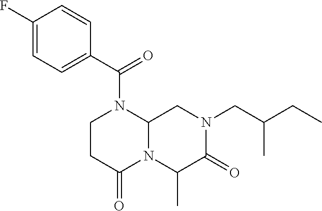 [SMILES: CCC(C)CN1CC2N(C(=O)c3ccc(F)cc3)CCC(=O)N2C(C)C1=O] |
| 12 | 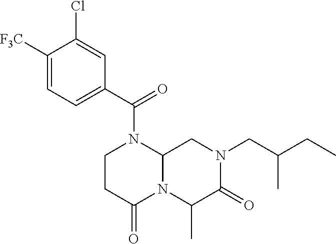 [SMILES: CCC(C)CN1CC2N(C(=O)c3ccc(C(F)(F)F)c(Cl)c3)CCC(=O)N2C(C)C1=O] |
| 14 | 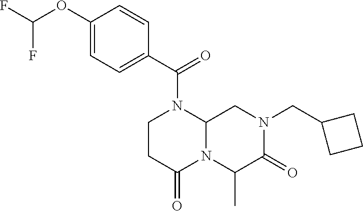 [SMILES: CC1C(=O)N(CC2CCC2)CC2N(C(=O)c3ccc(OC(F)F)cc3)CCC(=O)N12] |
| 17 | 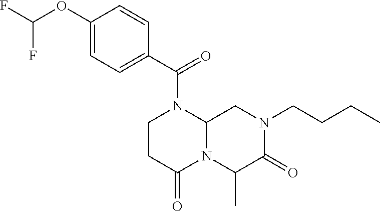 [SMILES: CCCCN1CC2N(C(=O)c3ccc(OC(F)F)cc3)CCC(=O)N2C(C)C1=O] |
| 19 | 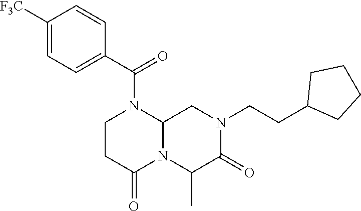 [SMILES: CC1C(=O)N(CCC2CCCC2)CC2N(C(=O)c3ccc(C(F)(F)F)cc3)CCC(=O)N12] |
| 20 | 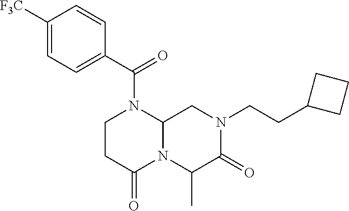 [SMILES: CC1C(=O)N(CCC2CCC2)CC2N(C(=O)c3ccc(C(F)(F)F)cc3)CCC(=O)N12] |
| 22 | 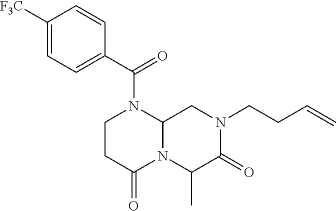 [SMILES: C=CCCN1CC2N(C(=O)c3ccc(C(F)(F)F)cc3)CCC(=O)N2C(C)C1=O] |
| 30 | 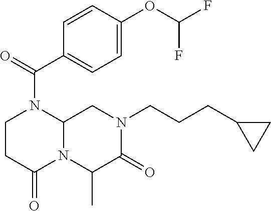 [SMILES: CC1C(=O)N(CCCC2CC2)CC2N(C(=O)c3ccc(OC(F)F)cc3)CCC(=O)N12] |
| 42 | 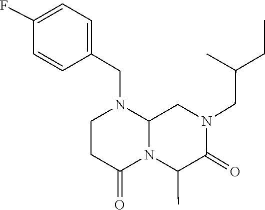 [SMILES: CCC(C)CN1CC2N(Cc3ccc(F)cc3)CCC(=O)N2C(C)C1=O] |
| 49 | 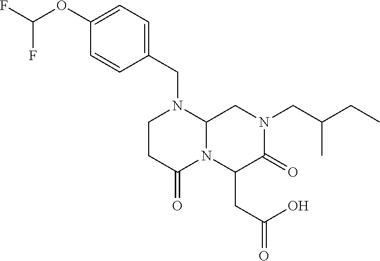 [SMILES: CCC(C)CN1CC2N(Cc3ccc(OC(F)F)cc3)CCC(=O)N2C(CC(=O)O)C1=O] |
| 51 | 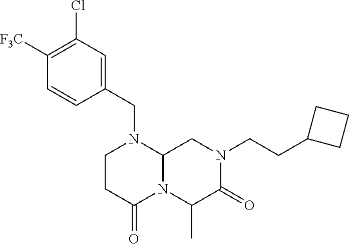 [SMILES: CC1C(=O)N(CCC2CCC2)CC2N(Cc3ccc(C(F)(F)F)c(Cl)c3)CCC(=O)N12] |
| 53 | 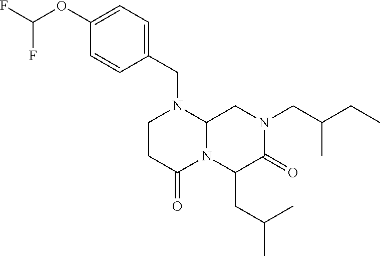 [SMILES: CCC(C)CN1CC2N(Cc3ccc(OC(F)F)cc3)CCC(=O)N2C(CC(C)C)C1=O] |

or a pharmaceutically acceptable salt thereof.

It is understood that in the present description, combinations of substituents and/or variables of the depicted formulae are permissible only if such contributions result in stable compounds.

Furthermore, all compounds of Formula (I) which exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

Compounds of Formula (I), or a pharmaceutically acceptable salt, isotopic form, or stereoisomer thereof, can be prepared by using organic chemistry synthesis methods known in the art. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

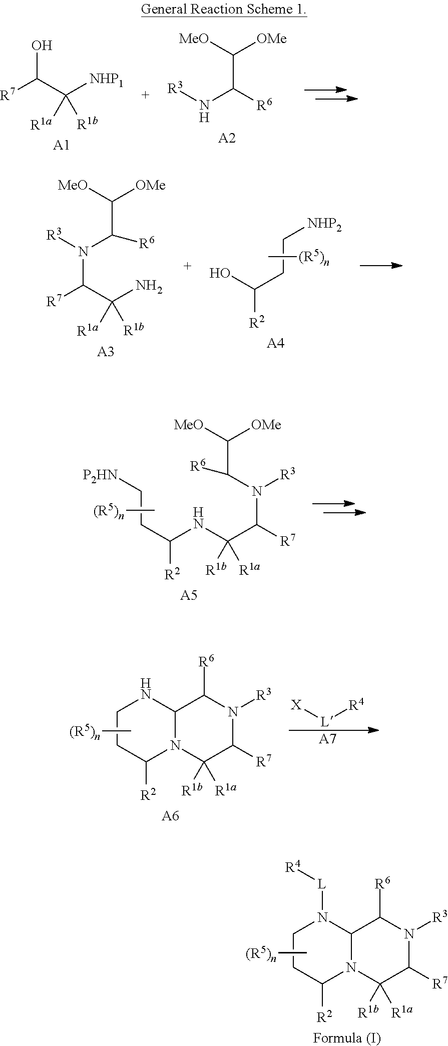

General Reaction Scheme 1 provides an exemplary method for preparation of compounds of Formula (I). R^{1a}, R^{1b}, R², R³, R⁴, R⁵, R⁶, R⁷, L, and n in General Reaction Scheme 1 are as defined herein. X is a reactive moiety selected to facilitate the desired reaction (e.g., halo). P₁ and P₂ are suitable protecting groups. L′ is selected such that a desired L moiety results from the reaction between L′-R⁴ and the secondary amine. Compounds of structure A1 are purchased or prepared according to methods known in the art. Reaction of A1 with A2 under appropriate coupling conditions (e.g., T3P and base) yields the product of the coupling reaction between A1 and A2, A3. A3 is then reacted with A4 under suitable coupling conditions (e.g., T3P and base) to afford compound A5. Compound A5 is then cyclized (e.g., using formic acid) and deprotected (e.g., using piperidine) to afford compound A6. Compound A6 is then reacted with compound A7 to afford the final compound of Formula (I) as shown.

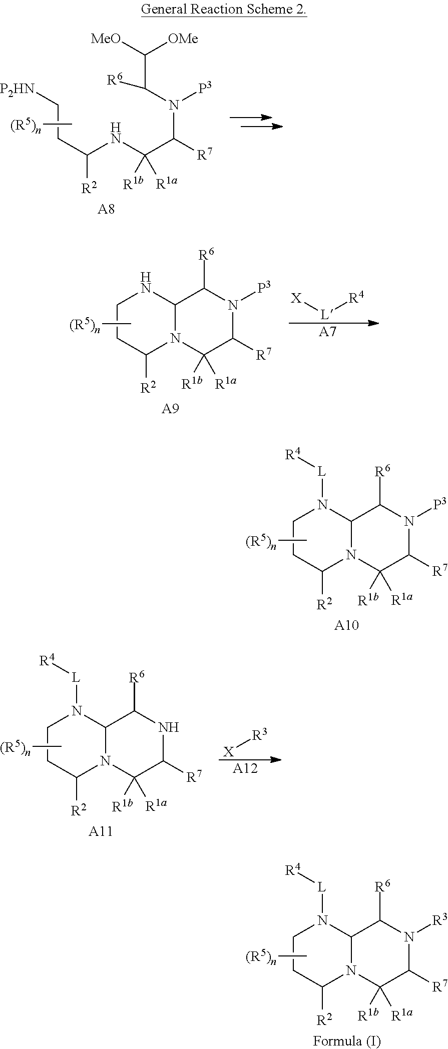

An alternative method for the synthesis of compounds of Formula (I) is depicted in General Reaction Scheme 2. R^{1a}, R^{1b}, R², R³, R⁴, R⁵, R⁶, R⁷, L, and n in General Reaction Scheme 2 are as defined herein. P₂ is a suitable protecting group. Each X is a reactive moiety selected to facilitate the desired reaction (e.g., halo). L′ is selected such that a desired L moiety results from the reaction between L′-R⁴ and the secondary amine. Intermediate A5 is prepared with a removable protecting group P³ (e.g. para-methoxybenzyl) as the R³ group giving intermediate A8. A8 is then cyclized (e.g., using formic acid) and deprotected (e.g., using piperidine) to afford compound A9. Compound A9 is then reacted with A7 to give compound A10. Compound A10 is then deprotected (e.g., with cerica ammonium nitrate) to give compound A11. Compound A11 is then reacted with A12 to provide the final compound of Formula (I).

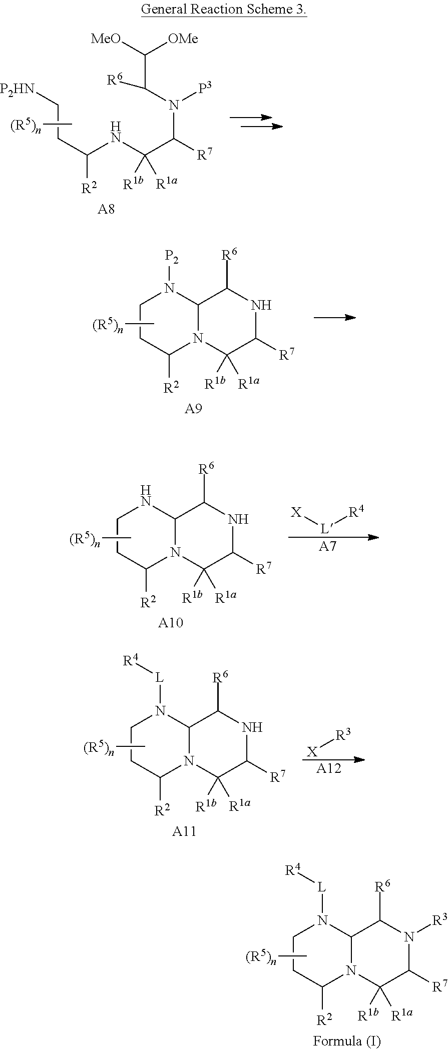

A related method to the one shown in General Reaction Scheme 2 is depicted in General Reaction Scheme 3. In this method, the two amine nitrogen atoms of the bicyclic core are deprotected to provide compound A10, then reacted with A7 to afford compound A11. Subsequent reaction with A12 provides the final compound of Formula (I).

It should be noted that various alternative strategies for preparation of compounds of Formula (I) are available to those of ordinary skill in the art. For example, other compounds of Formula (I) can be prepared according to analogous methods using the appropriate starting material.

It will also be appreciated by those skilled in the art that in the processes for preparing the compounds described herein the functional groups of intermediate compounds may need to be protected by suitable protecting groups. Such functional groups may include hydroxy, amino, and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino and amidino include t-butoxycarbonyl, benzyloxycarbonyl, and the like. Suitable protecting groups for carboxylic acid include alkyl, aryl, or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Pharmaceutical Compositions and Formulations

In a further aspect, provided herein are pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound of Formula (I), or a pharmaceutically acceptable salt, isotopic form, or stereoisomer thereof, and an additional therapeutic agent. Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound of Formula (I), or a pharmaceutically acceptable salt, isotopic form, or stereoisomer thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound of Formula (I), or a pharmaceutically acceptable salt, isotopic form, or stereoisomer thereof, is provided in the form of a rapid release formulation, in the form of an extended release formulation, or in the form of an intermediate release formulation. In yet other embodiments, the compound described herein is administered topically.

The compounds of Formula (I), or a pharmaceutically acceptable salt, isotopic form, or stereoisomer thereof, are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. An exemplary dosage is 10 to 30 mg per day. The exact dosage will depend upon the route of administration, the form in which the compound is administered, the subject to be treated, the body weight of the subject to be treated, and the preference and experience of the attending physician.

In some embodiments, a compound of Formula (I), or a pharmaceutically acceptable salt, isotopic form, or stereoisomer thereof, is administered in a single dose. Typically, such administration will be by injection, e.g., intravenous injection, in order to introduce the agent quickly. However, other routes are used as appropriate. A single dose of a compound of the disclosure may also be used for treatment of an acute condition (e.g., traumatic brain injury).

In some embodiments, a compound of Formula (I), or a pharmaceutically acceptable salt, isotopic form, or stereoisomer thereof, is administered in multiple doses. In some embodiments, dosing is about once, twice, three times, four times, five times, six times, or more than six times per day. In other embodiments, dosing is about once a month, once every two weeks, once a week, or once every other day. In another embodiment a compound of Formula (I), or a pharmaceutically acceptable salt, isotopic form, or stereoisomer thereof, and another therapeutic agent are administered together about once per day to about 6 times per day. In another embodiment the administration of a compound of Formula (I), or a pharmaceutically acceptable salt, isotopic form, or stereoisomer thereof, and a therapeutic agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of Formula (I), or a pharmaceutically acceptable salt, isotopic form, or stereoisomer thereof, may continue as long as necessary. In some embodiments, a compound of Formula (I), or a pharmaceutically acceptable salt, isotopic form, or stereoisomer thereof, is administered for more than 1, 2, 3, 4, 5, 6, 7, 14, or 28 days. In some embodiments, a compound of Formula (I), or a pharmaceutically acceptable salt, isotopic form, or stereoisomer thereof, is administered for less than 28, 14, 7, 6, 5, 4, 3, 2, or 1 day. In some embodiments, a compound of Formula (I), or a pharmaceutically acceptable salt, isotopic form, or stereoisomer thereof, is administered chronically on an ongoing basis, e.g., for the treatment of chronic effects (e.g., dementia).

In some embodiments, the compound of Formula (I), or a pharmaceutically acceptable salt, isotopic form, or stereoisomer thereof, is administered in dosages. It is known in the art that due to intersubject variability in compound pharmacokinetics, individualization of dosing regimen is necessary for optimal therapy. Dosing for a compound may be found by routine experimentation in light of the instant disclosure.

In some embodiments, the compounds Formula (I), or a pharmaceutically acceptable salt, isotopic form, or stereoisomer thereof, are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of Formula (I), or a pharmaceutically acceptable salt, isotopic form, or stereoisomer thereof, and a pharmaceutically acceptable diluent(s), excipient(s), or carrier(s). Also provided herein are methods for administering a pharmaceutical composition comprising a compound of Formula (I), or a pharmaceutically acceptable salt, isotopic form, or stereoisomer thereof, and a pharmaceutically acceptable diluent(s), excipient(s), or carrier(s).

In certain embodiments, the compounds are administered as pharmaceutical compositions in which compounds of Formula (I), or a pharmaceutically acceptable salt, isotopic form, or stereoisomer thereof, are mixed with other therapeutic agents, as in combination therapy. Encompassed herein are all combinations of active ingredients set forth in the methods section below and throughout this disclosure. In specific embodiments, the pharmaceutical compositions include one or more compounds of Formula (I), or a pharmaceutically acceptable salt, isotopic form, or stereoisomer thereof.

A pharmaceutical composition, as used herein, refers to a mixture of a compound of Formula (I), or a pharmaceutically acceptable salt, isotopic form, or stereoisomer thereof, with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Formula (I), or a pharmaceutically acceptable salt, isotopic form, or stereoisomer thereof, provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds of Formula (I), or a pharmaceutically acceptable salt, isotopic form, or stereoisomer thereof, are formulated in an aqueous solutions. In specific embodiments, the aqueous solution is selected from, by way of example only, a physiologically compatible buffer, such as Hank's solution, Ringer's solution, or physiological saline buffer. In other embodiments, one or more compound of Formula (I), or a pharmaceutically acceptable salt, isotopic form, or stereoisomer thereof, are formulated for transmucosal administration. In specific embodiments, transmucosal formulations include penetrants that are appropriate to the barrier to be permeated (e.g., the blood-brain barrier). In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds of Formula (I), or a pharmaceutically acceptable salt, isotopic form, or stereoisomer thereof, are formulated for oral administration. Compounds are formulated by combining the active compounds with, e.g., pharmaceutically acceptable carriers or excipients. In various embodiments, the compounds Formula (I), or a pharmaceutically acceptable salt, isotopic form, or stereoisomer thereof, are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with one or more of the compounds of Formula (I), or a pharmaceutically acceptable salt, isotopic form, or stereoisomer thereof, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds Formula (I), or a pharmaceutically acceptable salt, isotopic form, or stereoisomer thereof, are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds of Formula (I), or a pharmaceutically acceptable salt, isotopic form, or stereoisomer thereof, described herein are formulated for buccal or sublingual administration. Formulations suitable for buccal or sublingual administration include, by way of example only, tablets, lozenges, or gels. In still other embodiments, the compounds of Formula (I), or a pharmaceutically acceptable salt, isotopic form, or stereoisomer thereof, are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, a suspension of an active compound or compounds (e.g., compounds of Formula (I), or a pharmaceutically acceptable salt, isotopic form, or stereoisomer thereof,) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of Formula (I), or a pharmaceutically acceptable salt, isotopic form, or stereoisomer thereof, are administered topically. The compounds are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of Formula (I), or a pharmaceutically acceptable salt, isotopic form, or stereoisomer thereof, are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Formula (I), or a pharmaceutically acceptable salt, isotopic form, or stereoisomer thereof, is accomplished by means of iontophoretic patches and the like. In certain embodiments, transdermal patches provide controlled delivery of the compounds of Formula (I), or a pharmaceutically acceptable salt, isotopic form, or stereoisomer thereof. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of Formula (I), or a pharmaceutically acceptable salt, isotopic form, or stereoisomer thereof, are formulated for administration by inhalation. Various forms suitable for administration by inhalation include, but are not limited to, aerosols, mists or powders. Pharmaceutical compositions of any of compound of Formula (I), or a pharmaceutically acceptable salt, isotopic form, or stereoisomer thereof, are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, the compounds of Formula (I), or a pharmaceutically acceptable salt, isotopic form, or stereoisomer thereof, are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of Formula (I), or a pharmaceutically acceptable salt, isotopic form, or stereoisomer thereof, are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of Formula (I), or a pharmaceutically acceptable salt, isotopic form, or stereoisomer thereof, described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds Formula (I), or a pharmaceutically acceptable salt, isotopic form, or stereoisomer thereof, encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds of Formula (I), or a pharmaceutically acceptable salt, isotopic form, or stereoisomer thereof, include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound of Formula (I), or a pharmaceutically acceptable salt, isotopic form, or stereoisomer thereof. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of Formula (I), or a pharmaceutically acceptable salt, isotopic form, or stereoisomer thereof, illustratively takes the form of a liquid where the agents are present in solution, in suspension or both. Typically, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of Formula (I), or a pharmaceutically acceptable salt, isotopic form, or stereoisomer thereof. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite, and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds of Formula (I), or a pharmaceutically acceptable salt, isotopic form, or stereoisomer thereof, are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of the compound of Formula (I) provided in the pharmaceutical compositions of the present disclosure is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the compound of Formula (I) provided in the pharmaceutical compositions of the present disclosure is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the compound of Formula (I), or a pharmaceutically acceptable salt, isotopic form, or stereoisomer thereof, provided in the pharmaceutical compositions ranges from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, or approximately 1% to approximately 10% w/w, w/v or v/v.

In some embodiments, the concentration of the compound of Formula (I), or a pharmaceutically acceptable salt, isotopic form, or stereoisomer thereof, provided in the pharmaceutical compositions ranges from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, or approximately 0.1% to approximately 0.9% w/w, w/v or v/v.

In some embodiments, the amount the compound of Formula (I), or a pharmaceutically acceptable salt, isotopic form, or stereoisomer thereof, provided in the pharmaceutical compositions is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the compound of Formula (I), or a pharmaceutically acceptable salt, isotopic form, or stereoisomer thereof, provided in the pharmaceutical compositions of the present disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of the compound of Formula (I), or a pharmaceutically acceptable salt, isotopic form, or stereoisomer thereof, provided in the pharmaceutical compositions ranges from 0.0001-10 g, 0.0005-9 g, 0.001-8 g, 0.005-7 g, 0.01-6 g, 0.05-5 g, 0.1-4 g, 0.5-4 g, or 1-3 g.

### Kits/Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound of Formula (I), or a pharmaceutically acceptable salt, isotopic form, or stereoisomer thereof. Non-limiting examples of such materials include, but not limited to, buffers, diluents, filters, needles, syringes, carrier, package, container, vial, and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound of Formula (I), or a pharmaceutically acceptable salt, isotopic form, or stereoisomer thereof, formulated in a compatible pharmaceutical carrier are prepared, placed in an appropriate container, and labeled for treatment of an indicated condition.

### Methods of Use/Treatments

Embodiments of the present disclosure provide a method for modulating hepatocyte growth factor in a subject in need thereof, the method comprising administering to the subject an effective amount of a compound as disclosed herein (e.g., a compound of Formula (I), or a pharmaceutically acceptable salt, isotopic form, or stereoisomer thereof). In some embodiments, a compound described herein activates hepatocyte growth factor. Modulation (e.g., inhibition or activation) of hepatocyte growth factor can be assessed and demonstrated by a wide variety of ways known in the art. Kits and commercially available assays can be utilized for determining whether and to what degree hepatocyte growth factor has been modulated (e.g., inhibited or activated).

In some embodiments, provided herein are compounds of Formula (I), or a pharmaceutically acceptable salt thereof, for use in modulating hepatocyte growth factor in a subject in need thereof. In some embodiments, provided herein are compounds of Formula (I), or a pharmaceutically acceptable salt thereof, for the manufacture of a medicament for modulating hepatocyte growth factor in a subject in need thereof.

Applicant has discovered that the compounds Formula (I) show promising activity related to certain diseases of interest. Accordingly, in one aspect, provided herein is a method for modulating hepatocyte growth factor in a subject in need thereof, the method comprising administering to the subject an effective amount of a compound of Formula (I), or a pharmaceutically acceptable salt, isotopic form, or stereoisomer thereof. In some embodiments, provided herein is a method for activating hepatocyte growth factor in a subject in need thereof, the method comprising administering to the subject an effective amount of a compound of Formula (I), or a pharmaceutically acceptable salt, isotopic form, or stereoisomer thereof.

In certain more specific embodiments, the modulating comprises treating a disease, condition or injury.

In some embodiments, the disease, condition or injury is mild cognitive impairment. Mild cognitive impairment may be a precursor to dementia due to neurodegenerative disease, or may result from other factors, such as, for example, liver problems.

In some embodiments, a method of treating mild cognitive impairment in a subject in need thereof is provided, comprising administering to the subject an effective amount of a compound of Formula (I), or a pharmaceutically acceptable salt, isotopic form, or stereoisomer thereof.

Also provided herein is a method for treating or slowing progression of mild cognitive impairment in a subject in need thereof, the method comprising administering to the subject an effective amount of a compound of Formula (I), or a pharmaceutically acceptable salt, isotopic form, or stereoisomer thereof. Also provided herein is a method for treating or slowing progression of dementia in a subject with mild cognitive impairment, the method comprising administering to the subject an effective amount of a compound of Formula (I), or a pharmaceutically acceptable salt, isotopic form, or stereoisomer thereof.

In a further aspect, provided herein is a method for preventing cognitive dysfunction in a subject with mild cognitive impairment, the method comprising administering to the subject an effective amount of a compound of Formula (I), or a pharmaceutically acceptable salt, isotopic form, or stereoisomer thereof. In some embodiments, provided herein is a method for improving cognitive function and/or slowing progression of cognitive dysfunction in a subject with mild cognitive impairment, the method comprising administering to the subject an effective amount of a compound of Formula (I), or a pharmaceutically acceptable salt, isotopic form, or stereoisomer thereof.

Embodiments of the methods described above comprise administering to the mammal a therapeutically effective amount of a compound of Formula (I), or a pharmaceutically acceptable salt, isotopic form, or stereoisomer thereof. The methods disclosed herein are generally directed to administration of compounds of Formula (I), or a pharmaceutically acceptable salt, isotopic form, or stereoisomer thereof, to treat, protect from or reverse disease and injury associated with nerve cells or the nervous system. That is, embodiments of the present disclosure are directed to treatment, prevention or reversal of neurodegenerative diseases including treatment of dementia; repair of traumatic injury; and/or to prevent cognitive dysfunction. Certain embodiments of the present disclosure are directed to treatment, prevention or reversal of mild cognitive impairment, including treatment of dementia; repair of traumatic injury; improvement of cognitive function; and/or slowing progression of cognitive dysfunction.

In some embodiments, the disclosure provides methods of modulating protein activity (e.g., hepatocyte growth factor) in subject including but not limited to rodents and mammal (e.g., human) by administering into the subject an effective amount of a compound of Formula (I), or a pharmaceutically acceptable salt, isotopic form, or stereoisomer thereof. In some embodiments, modulation of hepatocyte growth factor is activation of hepatocyte growth factor. In some embodiments, the percentage modulation exceeds 25%, 30%, 40%, 50%, 60%, 70%, 80%, or 90%. In some embodiments, the percentage of inhibiting exceeds 25%, 30%, 40%, 50%, 60%, 70%, 80%, or 90%.

In some embodiments, the disclosure provides methods of modulating hepatocyte growth factor activity in a cell by contacting said cell with an amount of a compound of Formula (I), or a pharmaceutically acceptable salt, isotopic form, or stereoisomer thereof, sufficient to modulate the activity of hepatocyte growth factor. In some embodiments, the disclosure provides methods of modulating hepatocyte growth factor activity in a tissue by contacting said tissue with an amount of a compound of Formula (I), or a pharmaceutically acceptable salt, isotopic form, or stereoisomer thereof, sufficient to modulate the activity of hepatocyte growth factor in the tissue. In some embodiments, the disclosure provides methods of modulating hepatocyte growth factor activity in an organism by contacting said organism with an amount of a compound of Formula (I), or a pharmaceutically acceptable salt, isotopic form, or stereoisomer thereof, sufficient to modulate the activity of hepatocyte growth factor in the organism. In some embodiments, the disclosure provides methods of modulating hepatocyte growth factor activity in an animal by contacting the animal with an amount of a compound of Formula (I), or a pharmaceutically acceptable salt, isotopic form, or stereoisomer thereof, sufficient to modulate the activity of hepatocyte growth factor in the animal. In some embodiments, the disclosure provides methods of modulating hepatocyte growth factor activity in a mammal by contacting the mammal with an amount of a compound of Formula (I), or a pharmaceutically acceptable salt, isotopic form, or stereoisomer thereof, sufficient to modulate the activity of hepatocyte growth factor in the mammal. In some embodiments, the disclosure provides methods of modulating hepatocyte growth factor activity in a human by contacting the human with an amount of a compound of Formula (I), or a pharmaceutically acceptable salt, isotopic form, or stereoisomer thereof, sufficient to modulate the activity of hepatocyte growth factor in the human. In other embodiments, the present disclosure provides methods of treating a disease mediated by hepatocyte growth factor activity in a subject in need of such treatment. In some variations, modulation of hepatocyte growth factor by a compound of Formula (I), or a pharmaceutically acceptable salt, isotopic form, or stereoisomer thereof, involves activation of hepatocyte growth factor.

Other embodiments provide methods for combination therapies in which a therapeutic agent known to modulate other pathways, or other components of the same pathway, or even overlapping sets of target enzymes are used in combination with a compound of Formula (I), or a pharmaceutically acceptable salt, isotopic form, or stereoisomer thereof. In one aspect, such therapy includes but is not limited to the combination of one or more compounds of Formula (I), or a pharmaceutically acceptable salt, isotopic form, or stereoisomer thereof, with therapeutic agents, therapeutic antibodies, and other forms of treatment, to provide a synergistic or additive therapeutic effect.

Many therapeutic agents are presently known in the art and can be used in combination with the compounds of Formula (I), or a pharmaceutically acceptable salt, isotopic form, or stereoisomer thereof. In some embodiments, the therapeutic agent is selected from memantine, cholinesterase inhibitors, antidepressants, anxiolytics, and/or antipsychotic medicines. Some embodiments include use of therapies that include reminiscent therapy, cognitive stimulation therapy, reality orientation training, physical activity, and the like.

Exemplary cholinesterase inhibitors may include donepenzil, galantamine, and rivastigmine, which help to slow the breakdown of a brain chemical involved in memory and judgment. Memantine may help to control a different brain chemical needed for learning and memory. In certain aspects, memantine may also be used with donepezil in a combination drug for moderate to severe dementia. Antidepressants may include, but are not limited to, selective serotonin reuptake inhibitors (SSRIs). Anxiolytics may include, but are not limited to, lorazepam (Ativan) or oxazepam (Serax). Some embodiments of the methods described herein may include use or administration of antipsychotic medicines such as aripiprazole (Abilify), haloperidol (Haldol), olanzapine (Zyprexa), and risperidone (Risperdal).

In some embodiments, the compounds of Formula (I), or a pharmaceutically acceptable salt, isotopic form, or stereoisomer thereof, are formulated or administered in conjunction with liquid or solid tissue barriers also known as lubricants. Examples of tissue barriers include, but are not limited to, polysaccharides, polyglycans, seprafilm, interceed and hyaluronic acid.

In some embodiments, therapeutic agents that are administered in conjunction with the compounds of Formula (I), or a pharmaceutically acceptable salt, isotopic form, or stereoisomer thereof, include any suitable therapeutic agent usefully delivered by inhalation for example, analgesics, e.g. codeine, dihydromorphine, ergotamine, fentanyl, or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon. It will be clear to a person skilled in the art that, where appropriate, the therapeutic agents are used in the form of salts (e.g., as alkali metal or amine salts or as acid addition salts) or as esters (e.g., lower alkyl esters) or as solvates (e.g., hydrates) to optimize the activity and/or stability of the therapeutic agent.

Further therapeutic agents that can be combined with a compound of Formula (I), or a pharmaceutically acceptable salt, isotopic form, or stereoisomer thereof, are found in Goodman and Gilman's “The Pharmacological Basis of Therapeutics” Tenth Edition edited by Hardman, Limbird and Gilman or the Physician's Desk Reference, both of which are incorporated herein by reference in their entirety.

The compounds of Formula (I), or a pharmaceutically acceptable salt, isotopic form, or stereoisomer thereof, can be used in combination with the therapeutic agents disclosed herein depending on the condition being treated. Hence, in some embodiments the one or more compounds of Formula (I), or a pharmaceutically acceptable salt, isotopic form, or stereoisomer thereof, will be co-administered with other therapeutic agents as described above. When used in combination therapy, the compounds of Formula (I), or a pharmaceutically acceptable salt, isotopic form, or stereoisomer thereof, are administered with the second therapeutic agent simultaneously or separately. This administration in combination can include simultaneous administration in the same dosage form, simultaneous administration in separate dosage forms, and separate administration. That is, a compound of Formula (I), or a pharmaceutically acceptable salt, isotopic form, or stereoisomer thereof, and any of the therapeutic agents described above can be formulated together in the same dosage form and administered simultaneously. Alternatively, a compound of Formula (I), or a pharmaceutically acceptable salt, isotopic form, or stereoisomer thereof, and any of the therapeutic agents described above can be simultaneously administered, wherein both are present in separate formulations. In another alternative, a compound of Formula (I), or a pharmaceutically acceptable salt, isotopic form, or stereoisomer thereof, can be administered just followed by and any of the therapeutic agents described above, or vice versa. In some embodiments of the separate administration protocol, a compound of Formula (I), or a pharmaceutically acceptable salt, isotopic form, or stereoisomer thereof, and any of the therapeutic agents described above are administered a few minutes apart, or a few hours apart, or a few days apart.

The examples and preparations provided below further illustrate and exemplify the compounds of Formula (I), or a pharmaceutically acceptable salt, isotopic form, or stereoisomer thereof, and methods of preparing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

### Examples

The following example are provided for exemplary purposes. Methods for preparation of compounds of Formula (I), or a pharmaceutically acceptable salt, isotopic form, or stereoisomer thereof, are provided herein or can be derived by one of ordinary skill in the art.

The examples and preparations provided below further illustrate and exemplify the compounds of the present disclosure and methods for testing such compounds. It is to be understood that the scope of the present disclosure is not limited in any way by the scope of the following examples.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be performed by modifications apparent to those skilled in the art, for example by appropriately protecting interfering groups, by utilizing other suitable reagents known in the art other than those described, or by making routine modification of reaction conditions, reagents, and starting materials. Alternatively, other reactions disclosed herein or known in the art will be recognized as having applicability for preparing other compounds of the present disclosure.

Unless indicated otherwise in the following Examples, the compounds are isolated as a racemic mixture.

The following abbreviations may be relevant for the application.

### Abbreviations

AcOH: acetic acid
CAN: ceric ammonium nitrate
DAST: diethylaminosulfur trifluoride
DCM: dichloromethane
DIPEA: N,N-diisopropylethylamine
DMEM: Dulbecco's Modified Eagle Medium
DMF: dimethylformamide
DMSO: dimethylsulfoxide
EMEM: Eagle's Minimum Essential Medium
EtOAc: ethyl acetate
EtOH: ethanol
FBS: fetal bovine serum
Fmoc: fluorenylmethoxycarbonyl
HATU: (1-[bis(dimethylamino)methylene]-1H-1,2,3-triazolo[4,5-b]pyridinium 3-oxide hexafluorophosphate
LC/MS: liquid chromatography-mass spectrometry
Me: methyl
MeOH: methanol
PBS: phosphate buffered saline
Pic-BH₃: picoline borane
PMB: para-methoxybenzyl ether
Prep HPLC: preparative high performance liquid chromatography
rt or RT: room temperature
TFA: trifluoroacetic acid
TLC: thin layer chromatography
T3P: Propanephosphonic acid anhydride

### Synthetic Examples

### Example S1. Synthesis of (6S)-6-methyl-8-(2-methylbutyl)hexahydro-4H-pyrazino[1,2-a]pyrimidine-4,7(6H)-dione

The synthetic route for preparing this starting material compound is shown in Scheme 1.

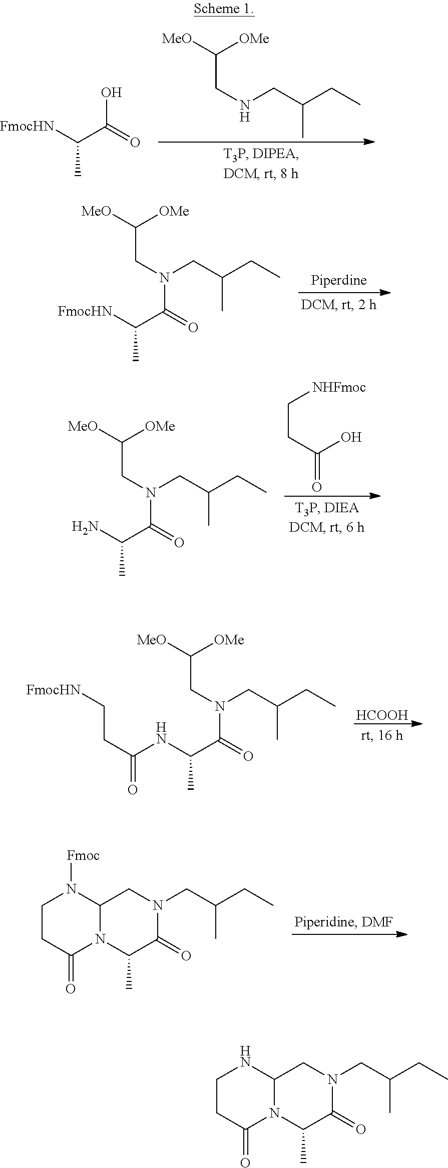

Step 1: Synthesis of (9H-fluoren-9-yl)methyl (2S)-1-((2,2-dimethoxyethyl)(2-methylbutyl)amino)-1-oxopropan-2-ylcarbamate. To a stirred solution of compound (S)-2-(((9H-fluoren-9-yl)methoxy)carbonylamino)propanoic acid (5.0 g, 16.07) in dichloromethane (100 mL) was added T3P (15.2 mL, 24.1) and DIPEA (5.6 mL, 32.1 mmol) at room temperature. The reaction mixture was stirred at room temperature for 15 min and N-(2,2-dimethoxyethyl)-2-methylbutan-1-amine (2.81 g, 32.1 mmol.) was added, and stirring was continued at room temperature for 8 hours. The reaction was monitored by TLC. After completion, the reaction mixture was quenched with ice cold water (100 mL) and extracted with dichloromethane (2×100 mL). The combined organic layers were dried over anhydrous Na₂SO₄, filtered and concentrated under reduced pressure to give crude compound. The crude compound was purified by flash column chromatography (100-200 mesh silica gel, eluted with 40% ethyl acetate in petroleum ether) to afford pure compound (9H-fluoren-9-yl)methyl (2S)-1-((2,2-dimethoxyethyl)(2-methylbutyl)amino)-1-oxopropan-2-ylcarbamate (5.2 g, 69.1%) as a gummy compound.

Step 2: Synthesis of (2S)-2-amino-N-(2,2-dimethoxyethyl)-N-(2-methylbutyl)propenamide. To a stirred solution of (9H-fluoren-9-yl)methyl (2S)-1-((2,2-dimethoxyethyl)(2-methylbutyl)amino)-1-oxopropan-2-ylcarbamate (34.0 g, 72.6 mmol) in DMF (230 mL) was added 20% piperidine in DMF (70 mL) at 0° C. The reaction mixture was stirred at room temperature for 2 hours. The reaction was monitored by TLC. After completion of the reaction, excess DMF (100 mL) was added, then washed with excess n-hexane (3×200 mL). The DMF layer was collected and poured in ice cold water (1000 mL), then extracted with 10% methanol-dichloromethane (3×500 mL). The combined organic layers was dried over anhydrous Na₂SO₄, filtered and concentrated under reduced pressure to give (2S)-2-amino-N-(2,2-dimethoxyethyl)-N-(2-methylbutyl)propanamide (20.4 g, 68.4%) as a gummy solid.

Step 3: Synthesis of (9H-fluoren-9-yl)methyl3-((2S)-1-((2,2-dimethoxyethyl)(2-methylbutyl)amino)-1-oxopropan-2-ylamino)-3-oxopropylcarbamate. To a stirred solution of 3-(((9H-fluoren-9-yl) methoxy)carbonylamino)propanoic acid (20.2 g, 81.2 mmol) stirred in dichloromethane at room temperature (500 mL) was added T3P (80 mL, 121.8 mmol) and DIPEA (28.6 mL, 160.4 mmol), and the mixture was stirred for 10 minutes. To this (2S)-2-amino-N-(2,2-dimethoxyethyl)-N-(2-methylbutyl)propanamide (25.53 81.2 mmol) was added and stirring was continued at room temperature for 16 hours. The reaction progress was monitored by TLC. After completion, the reaction mixture was quenched with water (500 mL) and the mixture was extracted with dichloromethane (2×500 mL). The combined organic layers were dried over Na₂SO₄, filtered and concentrated under reduced pressure to give crude product. The crude compound was purified by flash column chromatography (100-200 mesh Silica gel, eluted with 70% ethyl acetate in petroleum ether) to afford pure compound (9H-fluoren-9-yl)methyl3-((2S)-1-((2,2-dimethoxyethyl)(2-methylbutyl)amino)-1-oxopropan-2-ylamino)-3-oxopropylcarbamate (21.2 g, 78.6%) as a gummy compound.

Step 4: Synthesis of (6S)-(9H-fluoren-9-yl)methyl 6-methyl-8-(2-methylbutyl)-4,7-dioxooctahydro-1H-pyrazino[1,2-a]pyrimidine-1-carboxylate. To a stirred solution of (9H-fluoren-9-yl)methyl 3-((2S)-1-((2,2-dimethoxyethyl)(2-methylbutyl)amino)-1-oxopropan-2-ylamino)-3-oxopropylcarbamate (21.0 g, 38.9 mmol) was added formic acid (105 mL). The reaction mixture was stirred at room temperature for 12 hours. The reaction progress was monitored by TLC. After completion, the reaction mixture was concentrated under reduced pressure to give crude compound. The crude compound was taken up in saturated aqueous NaHCO₃ (200 mL) solution, then extracted with ethyl acetate (3×500 mL). The combined organic layers were washed with brine solution (500 mL), then the combined organic layers were dried over anhydrous Na₂SO₄, filtered and concentrated under reduced pressure. The crude compound was purified by flash column chromatography (100-200 mesh silica gel, eluted with 50% ethyl acetate in petroleum ether) to afford pure compound (6S)-(9H-fluoren-9-yl)methyl 6-methyl-8-(2-methylbutyl)-4,7-dioxooctahydro-1H-pyrazino[1,2-a]pyrimidine-1-carboxylate (25 g, 69.0%) as a gum.

Step 5: Synthesis of (6S)-6-methyl-8-(2-methylbutyl)tetrahydro-1H-pyrazino[1,2-a]pyrimidine-4,7(6H,8H)-dione. To a stirred solution of (6S)-(9H-fluoren-9-yl)methyl 6-methyl-8-(2-methylbutyl)-4,7-dioxooctahydro-1H-pyrazino[1,2-a]pyrimidine-1-carboxylate (14.0 g, 29.4 mmol) at 0° C. in DMF (70 mL) was added 20% piperidine in DMF (30 mL). The reaction mixture was allowed to warm to room temperature and stirred for 2 hours. The reaction was monitored by TLC. After complete consumption of starting material, additional DMF was added (50 mL), then the mixture was washed with excess n-hexane (3×200 mL). The DMF layer was poured into ice cold water (1000 mL) and extracted with 10% methanol-dichloromethane (3×500 mL). The combined organic layers were dried over anhydrous Na₂SO₄, filtered and concentrated under reduced pressure to provide the desired crude compound (6S)-6-methyl-8-(2-methylbutyl)tetrahydro-1H-pyrazino[1,2-a]pyrimidine-4,7(6H,8H)-dione (6.25 g, 83.8%) as a solid.

### Example S2. Synthesis of Compound 1a

The synthetic route for preparing Compound 1a is shown in Scheme 2.

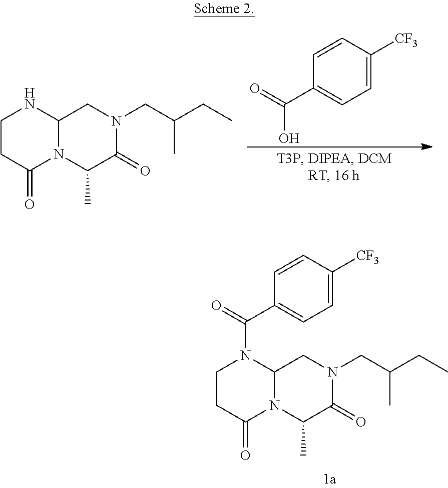

To a solution of 4-(trifluoromethyl)benzoic acid (0.232 g, 0.91 mmol) stirred in dichloromethane (20 mL) at room temperature was added T3P (1.2 mL, 1.37 mmol) and DIPEA (0.42 mL, 1.82 mmol), and the mixture was stirred for 15 minutes. To this (6S)-6-methyl-8-(2-methylbutyl)tetrahydro-1H-pyrazino[1,2-a]pyrimidine-4,7(6H,8H)-dione (0.310 g, 0.91 mmol) was added and stirring was continued for 8 hours. The reaction progress was monitored by TLC. After reaction completion, the mixture was quenched with water (50 mL) and extracted with dichloromethane (2×50 mL). The combined organic layers were dried over Na₂SO₄, filtered and concentrated under reduced pressure. The crude compound was purified by Prep HPLC. The pure fractions were combined and concentrated under reduced pressure, then lyophilized to afford 1a (0.340 g, 65.3%) as a solid. Prep HPLC method: Mobile phase A: 10 mM ammonium bicarbonate in water; Mobile phase B: acetonitrile; Column: X-Select phenyl hexyl (150×19 mm 5μ); Flow: 16 mL/min. MS (ESI) m/z [M+H]⁺: 426.05.

### Example S3. Synthesis of Compound 2a

The synthetic route for preparing Compound 2a is shown in Scheme 3.

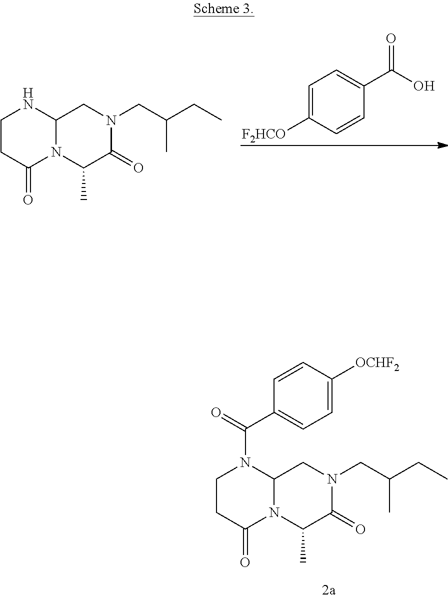 [SMILES: CCC(C)CN1CC2NCCC(=O)N2[C@@H](C)C1=O ; O=C(O)c1ccc(OC(F)F)cc1 ; CCC(C)CN1CC2N(C(=O)c3ccc(OC(F)F)cc3)CCC(=O)N2[C@@H](C)C1=O]

To a solution of 4-(difluoromethoxy) benzoic acid (0.37 g, 1.968 mmol) in dichloromethane (15 mL) at room temperature was added DIPEA (0.8 ml, 5.904 mmol) and T3P (2.0 mL, 3.936 mmol). The mixture was stirred for 30 min, then (6S)-6-methyl-8-(2-methylbutyl)hexahydro-4H-pyrazino[1,2-a]pyrimidine-4,7(6H)-dione (0.4 g, 1.578 mmol) was added, and stirring was continued for 16 hours. Progress of the reaction was monitored by TLC and LC/MS. The reaction mixture was diluted with dichloromethane (100 mL) and washed with water (50 mL) and saturated sodium chloride solution (50 mL), then dried over sodium sulfate, filtered and concentrated under reduced pressure. The crude product was purified by Prep HPLC. The pure fractions were collected and lyophilized to afford 2a (380 mg 46%) as a solid. Prep HPLC condition: Mobile phase A: 10 mM ammonium bicarbonate in water; Mobile phase B: Acetonitrile; Column: Kromosil phenyl (150×25 mm 10μ); Flow: 25 mL/min. MS (ESI) m/z [M+H]⁺: 424.11.

### Example S4. Synthesis of Compound 3a

The synthetic route for preparing Compound 3a is shown in Scheme 4.

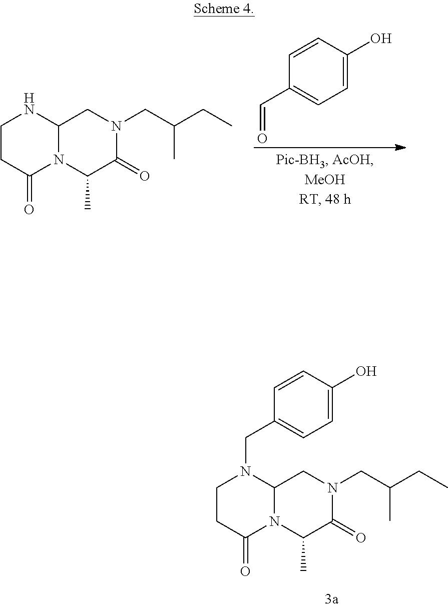

To a solution of (6S)-6-methyl-8-(2-methylbutyl)tetrahydro-1H-pyrazino[1,2-a]pyrimidine-4,7(6H,8H)-dione (0.500 g, 1.97 mmol) stirred in methanol (20 mL) at room temperature was added 4-hydroxybenzaldehyde (0.289 g, 1.97 mmol) and acetic acid (0.23 mL, 3.95 mmol). The reaction mixture was stirred at room temperature for 5 minutes. To this picoline borane (0.253 g, 2.37 mmol) was added, and stirring was continued for 48 hr. The reaction progress was monitored by TLC. After completion, the reaction mixture was quenched with ice cold water (50 mL), and the mixture was extracted with 10% methanol-dichloromethane (3×40 mL). The combined organic layers were dried over Na₂SO₄, filtered and concentrated under reduced pressure. The crude compound was purified by Prep HPLC. The pure fractions were combined and concentrated under reduced pressure, then lyophilized to give 3a (0.180 g, 46.09%) as a solid. Prep HPLC Method: Mobile Phase A: 10 mM ammonium bicarbonate in water; Mobile Phase B: Acetonitrile; Column: Kromosil Phenyl (150×25 mm 10μ); Flow: 25 mL/min. MS (ESI) m/z [M+H]⁺: 360.11.

### Example S5. Synthesis of Compound 4a

The synthetic route for preparing Compound 4a is shown in Scheme 5.

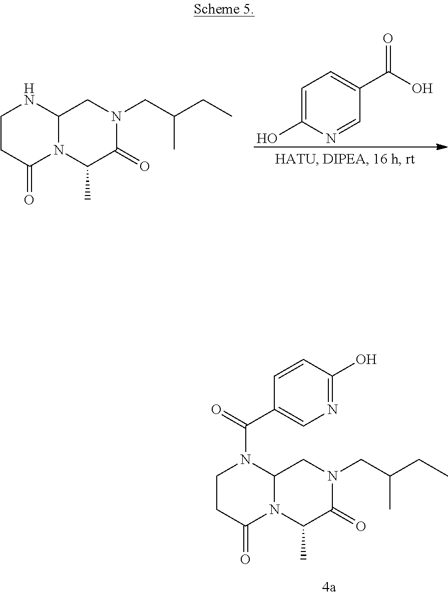

To a solution of 6-hydroxynicotinic acid (0.340 g 2.446 mmol) in DMF (15 mL) at room temperature was added DIPEA (1.30 mL, 7.338 mmol) and HATU (1.39 g, 3.669 mmol). The resulting reaction mixture was stirred for 30 min, then (6S)-6-methyl-8-(2-methylbutyl)hexahydro-4H-pyrazino[1,2-a]pyrimidine-4,7(6H)-dione (0.495 g, 1.956 mmol.) was added, and the mixture was stirred for 16 hours. Progress of the reaction was monitored by TLC and LC/MS (TLC system: 10% methanol/dichloromethane, Rf: 0.15, Detection: UV). The reaction mixture was quenched with cold water (100 mL) and extracted with 10% methanol/dichloromethane (3×100 mL).The combined organic layers were washed with cold water (50 mL) and cold brine solution (50 mL), dried over sodium sulfate, filtered and concentrated under reduced pressure. The crude product was purified by Prep HPLC. The pure fractions were collected and lyophilized to afford 4a (160 mg, 21.8%) as a solid. Prep HPLC Method: Mobile Phase A: 0.01 mM ammonium bicarbonate in water; Mobile Phase B: acetonitrile; Column: X-Select phenyl hexyl (150×19 mm, 5μ); Flow: 15 mL/min. MS (ESI) m/z [M+H]⁺: 375.05.

### Example S6. Synthesis of Compound 5a

The synthetic route for preparing Compound 5a is shown in Scheme 6.

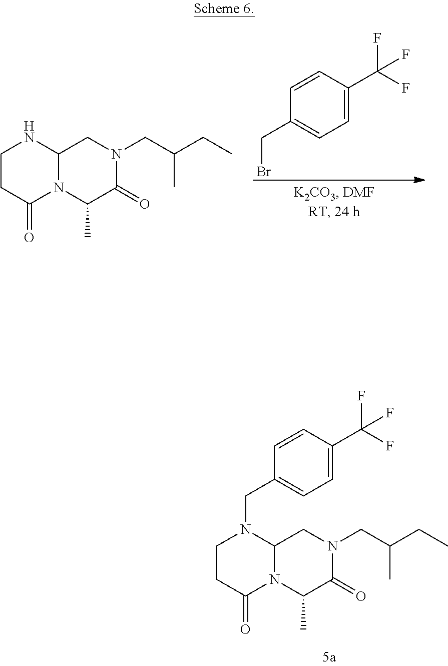

To a solution of (6S)-6-methyl-8-(2-methylbutyl)tetrahydro-1H-pyrazino[1,2-a]pyrimidine-4,7(6H,8H)-dione (0.5 g, 1.97 mmol) and 1-(bromomethyl)-4-(trifluoromethyl)benzene (0.470 g, 1.97 mmol) stirred in DMF (20 mL) at room temperature was added K₂CO₃ (0.546 g, 3.95 mmol), and the mixture was stirred for 8 hr. The reaction progress was monitored by TLC. After completion, the mixture was quenched with water (100 mL) and extracted with ethyl acetate (3×50 mL). The combined organic layers were dried over Na₂SO₄, filtered and concentrated under reduced pressure. The crude compound was purified by Prep HPLC. The pure fractions were combined and concentrated under reduced pressure, then lyophilized to afford 5a (0.270 g, 63.8%) as a gum. Prep HPLC Method: Mobile Phase A: 10 mM ammonium bicarbonate in water; Mobile Phase B: Acetonitrile; Column: Kromosil Cis (150×25 mm 10μ); Flow: 25 mL/min. MS (ESI) m/z [M+H]⁺: 412.2.

### Example S7. Synthesis of Compound 6a

The synthetic route for preparing Compound 6a is shown in Scheme 7.

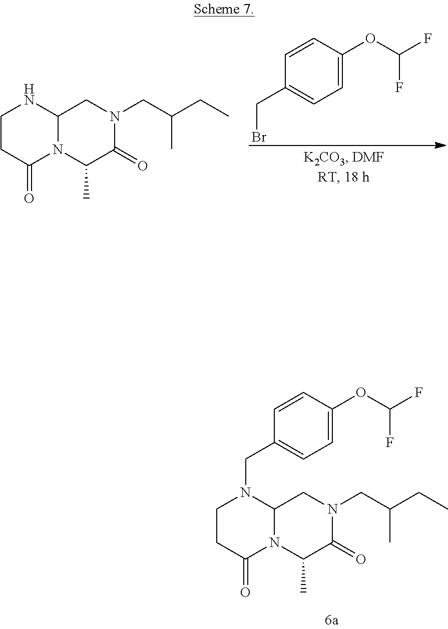

To a solution of (6S)-6-methyl-8-(2-methylbutyl)tetrahydro-1H-pyrazino[1,2-a]pyrimidine-4,7(6H,8H)-dione (0.500 g, 1.97 mmol) and 1-(bromomethyl)-4-(difluoromethoxy)benzene (0.466 g, 1.97 mmol) stirred in DMF (20 mL) at room temperature was added K₂CO₃ (0.546 g, 9.95 mmol). The reaction mixture was stirred at room temperature for 18 hr. The reaction progress was monitored by TLC. After completion, the reaction mixture was quenched with water (100 mL) and extracted with ethyl acetate (3×50 mL). The combined organic layers were dried over Na₂SO₄, filtered and concentrated under reduced pressure. The crude compound was purified by Prep HPLC. The pure fractions were combined and concentrated under reduced pressure, then lyophilized to afford 6a (0.178 g, 41.5%) as a semi-solid. Prep HPLC Method: Mobile Phase A: 10 mM ammonium bicarbonate in water; Mobile Phase B: acetonitrile; Column: X-Select Cis (250×19 mm, 5μ); Flow: 18 mL/min. MS (ESI) m/z [M+H]⁺: 410.11.

### Example S8. Synthesis of Compound 7a

The synthetic route for preparing Compound 7a is shown in Scheme 8.

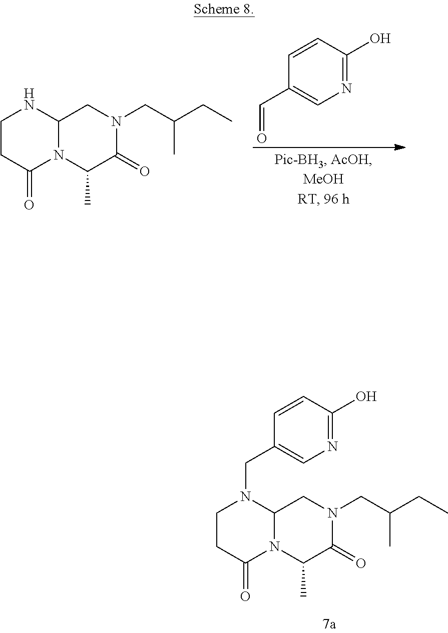

To a solution of compound (6S)-6-methyl-8-(2-methylbutyl)tetrahydro-1H-pyrazino[1,2-a]pyrimidine-4,7(6H,8H)-dione (0.500 g, 1.97 mmol) stirred in methanol (20 mL) at room temperature was added 6-hydroxynicotinaldehyde (0.243 g, 1.97 mmol) and acetic acid (0.25 mL, 3.95 mmol), and the mixture was stirred for 5 min. To this picoline borane (0.318 g, 2.96 mmol) was added and stirring was continued for 96 hours. The reaction progress was monitored by TLC. After completion, the reaction mixture was quenched with ice cold water (50 mL) and extracted with 10% methanol-dichloromethane (3×40 mL). The combined organic layers were dried over Na₂SO₄, filtered and concentrated under reduced pressure. The crude compound was purified by Prep HPLC. The pure fractions were collected and concentrated under reduced pressure, then lyophilized to afford 7a (0.164 g, 42%) as a solid. Prep HPLC Method: Mobile Phase A: 10 mM ammonium bicarbonate in water; Mobile Phase B: acetonitrile; Column: X-BRIDGE C₁₈ (250×19 mm, 5μ); Flow: 18 mL/min. MS (ESI) m/z [M+H]⁺: 361.11.

### Example S9. Synthesis of Compound 8a

The synthetic route for preparing Compound 8a is shown in Scheme 9.

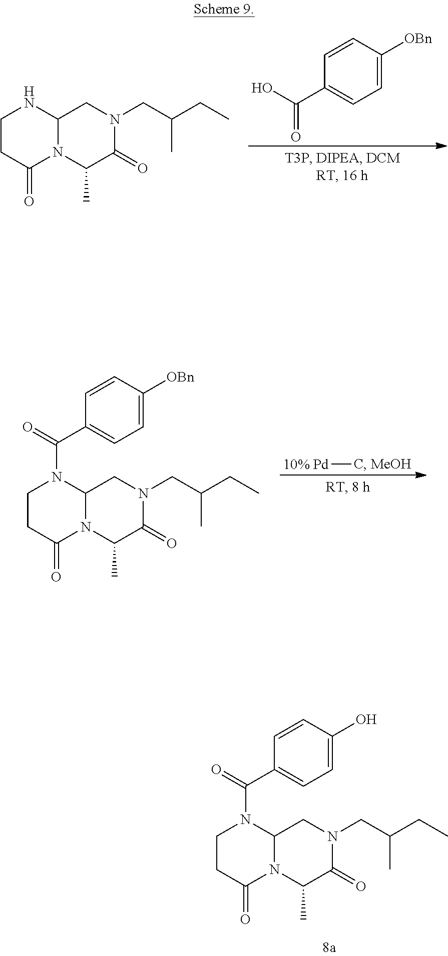

Step 1: Synthesis of (6S)-1-(4-(benzyloxy)benzoyl)-6-methyl-8-(2-methylbutyl)hexahydro-4H-pyrazino[1,2-a]pyrimidine-4,7(6H)-dione. To a solution of 4-(benzyloxy)benzoic acid (0.360 g, 1.42 mmol) stirred in dichloromethane (20 mL) at room temperature was added T3P (1.2 mL, 1.7 mmol) and DIPEA (0.55 mL, 2.84 mmol), and the mixture was stirred for 15 min. To this (6S)-6-methyl-8-(2-methylbutyl)tetrahydro-1H-pyrazino[1,2-a]pyrimidine-4,7(6H,8H)-dione (0.400 g, 1.42 mmol) was added, and stirring was continued at room temperature for 16 hours. The reaction progress was monitored by TLC. After completion, the reaction mixture was quenched with water (50 mL) and extracted with dichloromethane (2×50 mL). The combined organic layers were dried over Na₂SO₄, filtered and concentrated under reduced pressure to give 0.9 g of crude material. Analysis of the crude material by LC/MS showed 54.59% of the desired product. The crude material was used in the next step without purification.

Step 2: Synthesis of Compound 8a. To a solution of (6S)-1-(4-(benzyloxy)benzoyl)-6-methyl-8-(2-methylbutyl)tetrahydro-1H-pyrazino[1,2-a]pyrimidine-4,7(6H,8H)-dione (0.900 g) stirred in methanol (20 mL) at room temperature was added 10% Pd—C(0.200 g), under N2 atmosphere. The reaction mixture was stirred at room temperature under an H₂ balloon for 8 hr. The reaction progress was monitored by TLC. After completion, the reaction mixture was filtered through Celite and evaporated under reduced pressure to afford the crude compound. The crude compound was dissolved in dichloromethane (50 mL), washed with aqueous NaHCO₃ solution (20 mL) and brine solution (20 mL). The filtrate was dried over Na₂SO₄, filtered and concentrated under reduced pressure. The crude compound was triturated with diethyl ether to afford 8a (0.330 g, 82%) as a solid. MS (ESI) m/z [M+H]⁺: 374.11.

### Example S10. Synthesis of Compound 9

The synthetic route for preparing Compound 9 is shown in Scheme 10.

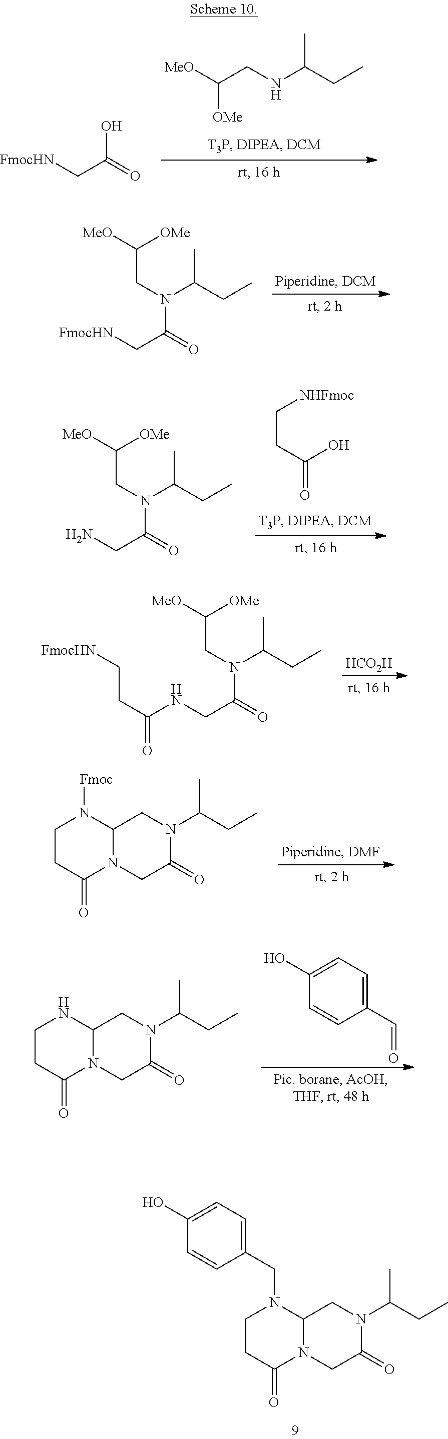

Step 1: Synthesis of (9H-fluoren-9-yl)methyl 2-(sec-butyl(2,2-dimethoxyethyl)amino)-2-oxoethylcarbamate. To a stirred solution of 2-(((9H-fluoren-9-yl)methoxy)carbonylamino)acetic acid (10 g, 33.6 mmol) in dichloromethane (100 mL), cooled to 0° C. were added DIPEA (11.88 mL, 67.3 mmol), N-(2,2-dimethoxyethyl)butan-2-amine (10.84 g, 67.3 mmol) and T3P (53.0 mL, 84.1 mmol), and the reaction mixture was stirred for 16 hours at room temperature. Reaction progress was monitored by TLC. After completion of the reaction, ice cold water (100 mL) was added and extracted with ethyl acetate (2×150 mL). The combined organic layers were dried over anhydrous Na₂SO₄, filtered and concentrated under reduced pressure to obtain the desired crude product. The crude compound was purified by flash column chromatography (100-200 mesh silica gel) and eluted with 20-25% ethyl acetate in petroleum ether to afford (9H-fluoren-9-yl)methyl 2-(sec-butyl(2,2-dimethoxyethyl)amino)-2-oxoethylcarbamate (10.8 g, 72.9%) as a solid.

Step 2: Synthesis of 2-amino-N-sec-butyl-N-(2,2-dimethoxyethyl)acetamide. To a solution of (9H-fluoren-9-yl)methyl 2-(sec-butyl(2,2-dimethoxyethyl)amino)-2-oxoethylcarbamate (10.8 g, 24.5 mmol) in DMF (20 mL), cooled to 0° C., was added piperidine (2.4 mL) and the reaction mixture was stirred at room temperature for 2 hours. Progress of the reaction was monitored by TLC. After TLC indicated complete consumption of starting material, the reaction mixture was diluted with petroleum ether (2×100 mL), then water was added and the mixture was separated. The aqueous layer was extracted with dichloromethane (2×150 mL). The combined organic layers were dried over anhydrous Na₂SO₄, filtered and concentrated under reduced pressure to obtain the desired pure product 2-amino-N-sec-butyl-N-(2,2-dimethoxyethyl)acetamide (3.6 g, 67.2%) as a solid.

Step 3: Synthesis of (9H-fluoren-9-yl)methyl-3-(2-(sec-butyl(2,2-dimethoxyethyl)amino)-2-oxoethylamino)-3-oxopropylcarbamate. To a stirred solution of 2-amino-N-sec-butyl-N-(2,2-dimethoxyethyl)acetamide (3.6 g, 16.5 mmol) in dichloromethane (40 mL) were added DIPEA (31.91 mL, 49.5 mmol), 3-(((9H-fluoren-9-yl)methoxy)carbonylamino)propanoic acid (5.14 g, 16.5 mmol) and T3P (39.13 g, 33 mmol) at 0° C. The reaction mixture was stirred at room temperature for 16 hours. Progress of the reaction was monitored by TLC. After completion of the reaction, the reaction water (100 mL) was added and the organic phase was separated. The aqueous phase was extracted with dichloromethane (2×150 mL). The combined organic layers were dried over Na₂SO₄, filtered and concentrated under reduced pressure to give the crude product. The crude product was purified by column chromatography using silica (230-400 mesh; 23-25% ethyl acetate/petroleum ether as eluent). Collected pure fractions were concentrated under reduced pressure to give (9H-fluoren-9-yl)methyl-3-(2-(sec-butyl(2,2-dimethoxyethyl)amino)-2-oxoethylamino)-3-oxopropylcarbamate (4.1 g, 48.6%) as a gum.

Step 4: Synthesis of (9H-fluoren-9-yl)methyl 8-sec-butyl-4,7-dioxooctahydro-1H-pyrazino[1,2-a]pyrimidine-1-carboxylate. To a solution of (9H-fluoren-9-yl)methyl-3-(2-(sec-butyl(2,2-dimethoxyethyl)amino)-2-oxoethylamino)-3-oxopropylcarbamate (4.1 g, 8.01 mmol) in acetic acid (2 mL) was stirred for 16 hours at room temperature. Progress of the reaction was monitored by TLC. After TLC indicated complete consumption of the starting material, the reaction mixture was concentrated and the resulting mass was diluted with water and extracted with dichloromethane (2×100 mL). The combined organic layers were dried over Na₂SO₄, filtered and concentrated under reduced pressure to give the product (9H-fluoren-9-yl)methyl 8-sec-butyl-4,7-dioxooctahydro-1H-pyrazino[1,2-a]pyrimidine-1-carboxylate. (3.2 g, 89.3%) as a gum.

Step 5: Synthesis of 8-sec-butyltetrahydro-1H-pyrazino[1,2-a]pyrimidine-4,7(6H,8H)-dione. To a solution of (9H-fluoren-9-yl)methyl 8-sec-butyl-4,7-dioxooctahydro-1H-pyrazino[1,2-a]pyrimidine-1-carboxylate (3.2 g, 7.1 mmol) in DMF (20 mL), cooled to 0° C., was added piperidine (0.7 mL, 1.0 eq) and the reaction mixture was stirred at room temperature for 2 hours. Progress of the reaction was monitored by TLC. After TLC indicated complete consumption of starting material, the reaction mixture was washed with petroleum ether (2×50 mL) to remove the non-polar impurities. Cold water was added and extracted with dichloromethane (2×100 mL). The combined organic layers were dried over Na₂SO₄, filtered and concentrated under reduced pressure to give the pure product 8-sec-butyltetrahydro-1H-pyrazino[1,2-a]pyrimidine-4,7(6H,8H)-dione (900 mg, 55.9%) as a solid.

Step 6: Synthesis of Compound 9. To a stirred solution of (8-(sec-butyl)hexahydro-4H-pyrazino[1,2-a]pyrimidine-4,7(6H)-dione (0.500 g, 2.2 mmol) and 4-hydroxybenzaldehyde (0.271 g, 2.2 mmol) in methanol (10 mL) was added acetic acid (0.27 mL, 2.0 eq.) and picoline borane (0.285 g, 2.6 mmol) at room temperature. The reaction mixture was stirred at room temperature for 48 hr. The reaction progress was monitored by TLC. After completion, the reaction mixture was quenched with ice cold water (10 mL) and extracted with ethyl acetate (2×20 mL). The combined organic layers were dried over Na₂SO₄, filtered and concentrated under reduced pressure to give crude product. The crude compound was analyzed by LC/MS. The crude LC/MS data showed 8.28% desired mass. The crude compound was purified by column chromatography over silica gel (100-200), and 50-70% ethyl acetate in petroleum ether eluted the desired compound. The LC/MS of the eluted fractions showed 72.16% desired mass, which was further purified by Prep HPLC. After Prep HPLC purification, the fractions were collected and concentrated under reduced pressure, then lyophilized to afford 9 (0.168 g, 22.8%) as a solid. Prep HPLC Method: Mobile Phase A: 10 mM ammonium bicarbonate in water; Mobile Phase B: acetonitrile; Column: X-BRIDGE Cis (150×19 mm 5μ); Flow: 18 mL/min. MS (ESI) m/z [M+H]⁺: 332.2.

### Example 511. Synthesis of Compound 10

The synthetic route for preparing Compound 10 is shown in Scheme 11.

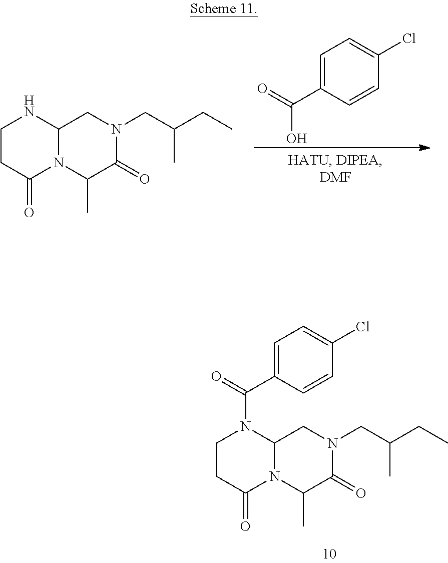 [SMILES: CCC(C)CN1CC2NCCC(=O)N2C(C)C1=O ; O=C(O)c1ccc(Cl)cc1 ; CCC(C)CN1CC2N(C(=O)c3ccc(Cl)cc3)CCC(=O)N2C(C)C1=O]

To a solution of 6-methyl-8-(2-methylbutyl)hexahydro-4H-pyrazino[1,2-a]pyrimidine-4,7(6H)-dione (250 mg, 0.98 mmol) and 4-chlorobenzoic acid (170 mg, 1.09 mmol) in DMF (4 mL) at 0° C. was added HATU (413 mg, 1.08 mmol) followed by DIPEA (0.35 mL, 1.97 mmol). The reaction mixture was stirred at room temperature for 12 h. After completion, the reaction mixture was quenched with ice cold water (50 mL) and the aqueous layer was extracted with EtOAc (50 mL×2). The organic layer was washed with cold H₂O (30 mL) followed by saturated brine (30 mL), dried over Na₂SO₄ and concentrated under reduced pressure. The crude obtained was purified by column chromatography (Silica 100-200 mesh; 30% EtOAc in hexanes) to afford 1-(4-chlorobenzoyl)-6-methyl-8-(2-methylbutyl)hexahydro-4H-pyrazino[1,2-a]pyrimidine-4,7(6H)-dione 10 (150 mg, 0.383 mmol, 39.2% yield) as a solid. MS (ESI) m/z [M+H]⁺: 392.05. ¹H NMR (400 MHz, DMSO-d6) δ 0.66-0.89 (m, 6H) 0.91-1.42 (m, 4H) 1.57-1.78 (m, 1H) 2.16-2.35 (m, 2H) 2.55-2.65 (m, 2H) 3.08-3.23 (m, 2H) 3.28-3.40 (m, 1H) 3.51-3.64 (m, 2H) 4.76-4.89 (m, 1H) 5.88-6.02 (m, 1H) 7.46-7.56 (m, 4H). Example S12. Synthesis of Compound 11. The synthetic route for preparing Compound 11 is shown in Scheme 12.

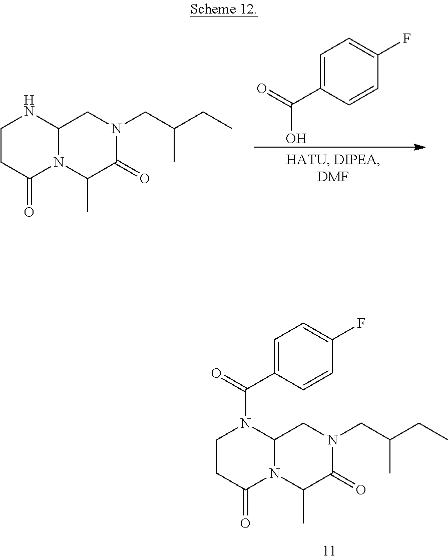 [SMILES: CCC(C)CN1CC2NCCC(=O)N2C(C)C1=O ; O=C(O)c1ccc(F)cc1 ; CCC(C)CN1CC2N(C(=O)c3ccc(F)cc3)CCC(=O)N2C(C)C1=O]

To a solution of 6-methyl-8-(2-methylbutyl)hexahydro-4H-pyrazino[1,2-a]pyrimidine-4,7(6H)-dione (250 mg, 0.98 mmol) and 4-fluorobenzoic acid (153 mg, 1.09 mmol) in DMF (4 mL) at 0° C. was added HATU (413 mg, 1.08 mmol) followed by DIPEA (0.35 mL, 1.97 mmol). The reaction mixture was stirred at room temperature for 12 h. After completion, the reaction mixture was quenched with ice cold water (50 mL) and the aqueous layer was extracted with EtOAc (50 mL×2). The organic layer was washed with cold H₂O (30 mL) followed by saturated brine (30 mL), dried over Na₂SO₄ and concentrated under reduced pressure. The crude obtained was purified by column chromatography (Silica 100-200 mesh; 30% EtOAc in hexanes) to afford 1-(4-fluorobenzoyl)-6-methyl-8-(2-methylbutyl)hexahydro-4H-pyrazino[1,2-a]pyrimidine-4,7(6H)-dione 11 (140 mg, 0.37 mmol, 38.0% yield) as a solid. MS (ESI) m/z [M+H]⁺: 376.05. ¹H NMR (400 MHz, DMSO-d6) δ 0.69-0.81 (m, 3H) 0.86 (t, J=7.23 Hz, 3H) 0.95-1.14 (m, 2H) 1.20-1.43 (m, 4H) 1.59-1.80 (m, 2H) 2.26 (d, J=16.95 Hz, 1H) 2.55-2.72 (m, 1H) 3.20-3.31 (m, 2H) 3.35-3.39 (m, 1H) 3.52-3.70 (m, 2H) 4.73-4.89 (m, 1H) 7.33 (t, J=8.73 Hz, 2H) 7.61 (dd, J=8.23, 5.73 Hz, 2H).

### Example S13. Synthesis of Compound 12

The synthetic route for preparing Compound 12 is shown in Scheme 13.

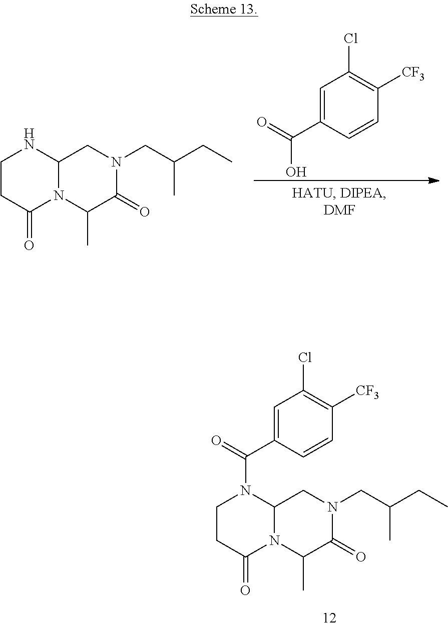 [SMILES: CCC(C)CN1CC2NCCC(=O)N2C(C)C1=O ; O=C(O)c1ccc(C(F)(F)F)c(Cl)c1 ; CCC(C)CN1CC2N(C(=O)c3ccc(C(F)(F)F)c(Cl)c3)CCC(=O)N2C(C)C1=O]

To a solution of 6-methyl-8-(2-methylbutyl)hexahydro-4H-pyrazino[1,2-a]pyrimidine-4,7(6H)-dione (250 mg, 0.98 mmol) and 3-chloro-4-(trifluoromethyl)benzoic acid (242 mg, 1.09 mmol) in DMF (4 mL) at 0° C. was added HATU (413 mg, 1.08 mmol) followed by DIPEA (0.35 mL, 1.97 mmol). The reaction mixture was stirred at room temperature for 12 h. After completion, the reaction mixture was quenched with ice cold water (50 mL) and the aqueous layer was extracted with EtOAc (50 mL×2). The organic layer was washed with cold H₂O (30 mL) followed by saturated brine (30 mL), dried over Na₂SO₄ and concentrated under reduced pressure. The crude obtained was purified by column chromatography (Silica 100-200 mesh; 30% EtOAc in hexanes) to afford 1-(3-chloro-4-(trifluoromethyl)benzoyl)-6-methyl-8-(2-methylbutyl)hexahydro-4H-pyrazino[1,2-a]pyrimidine-4,7(6H)-dione 12 (250 mg, 0.55 mmol, 55.2% yield) as a solid. MS (ESI) m/z [M+H]⁺: 460.0. ¹H NMR (400 MHz, DMSO-d6) δ 0.74-0.93 (m, 6H) 0.98-1.19 (m, 2H) 1.28-1.46 (m, 3H) 1.64-1.81 (m, 1H) 2.22 (d, J=17.45 Hz, 1H) 2.57-2.70 (m, 1H) 3.14 (dd, J=13.21, 6.23 Hz, 1H) 3.25-3.31 (m, 2H) 3.44-3.57 (m, 1H) 3.61-3.87 (m, 2H) 4.78-4.90 (m, 1H) 5.89-6.05 (m, 1H) 7.72 (d, J=7.98 Hz, 1H) 7.90-8.02 (m, 2H).

### Example S14. Synthesis of Intermediate Compound 8-(4-methoxybenzyl)-6-methylhexahydro-4H-pyrazino[1,2-a]pyrimidine-4,7(6H)-dione

The synthetic route for preparing this intermediate compound is shown in Scheme 14.

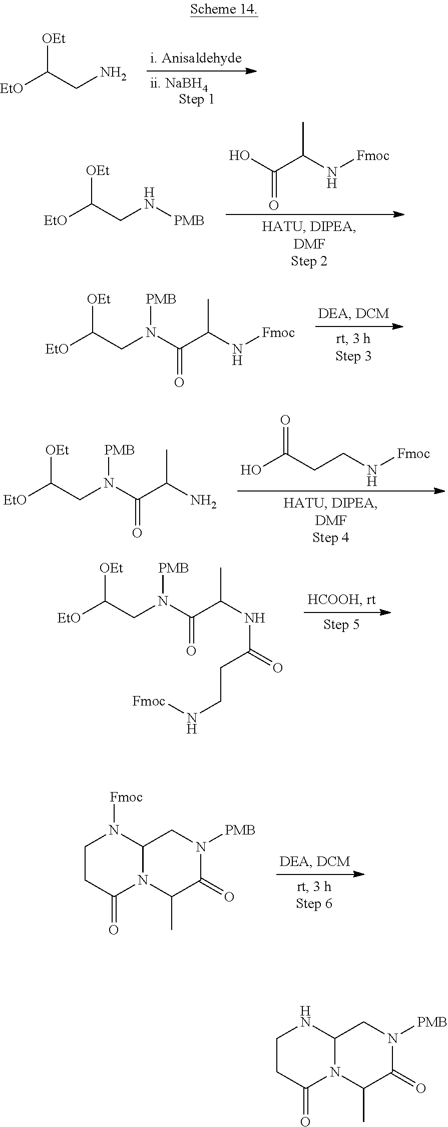

Step 1: Synthesis of 2,2-diethoxy-N-(4-methoxybenzyl)ethan-1-amine. A 500 mL round bottom flask was charged with anisaldehyde (12 mL, 90.22 mmol) and 2,2-diethoxyethanamine (10 g, 75.18 mmol). The reaction mixture was heated at 100° C. for 1 h. The reaction mixture was allowed to cool at room temperature and to this was added EtOH (100 mL) followed by NaBH₄ (4.28 g, 112.7 mmol). The resulting reaction mixture was stirred at room temperature for 16 h. After complete consumption of starting material (monitored by TLC), the reaction mixture was concentrated under reduced vacuum. The crude obtained was dissolved in EtOAc (300 mL). The organic layer was washed with brine (100 mL), dried over Na₂SO₄ and concentrated under vacuum to give crude product. The crude product obtained was purified by column chromatography (silica 100-200 mesh; 70% EtOAc in hexanes) to obtain 2,2-diethoxy-N-(4-methoxybenzyl)ethan-1-amine (15 g, 59.28 mmol, 78% yield) as a liquid. MS (ESI) m/z [M+H]⁺: 254.3.

Step 2: (9H-fluoren-9-yl)methyl (1-((2,2-diethoxyethyl)(4-methoxybenzyl)amino)-1-oxopropan-2-yl)carbamate. To a stirred solution of (((9H-fluoren-9-yl)methoxy)carbonyl)alanine (32 g, 102.76 mmol) in dry DMF (140 mL) maintained at 0° C. was added HATU (42 g, 110.67 mmol), DIPEA (21.06 mL, 118.57 mmol), followed by 2,2-diethoxy-N-(4-methoxybenzyl)ethan-1-amine (20 g, 79.05 mmol). The reaction mixture was stirred at room temperature for 16 h. After complete consumption of starting material, the reaction mixture was quenched with ice cold water (300 mL) and the aqueous layer was extracted with EtOAc (200 mL×2). The organic layer was washed with cold H₂O (200 mL) followed by brine (100 mL), dried over Na₂SO₄ and concentrated under reduced pressure to give crude product. The crude obtained was purified by column chromatography (Silica 100-200 mesh; 50% EtOAc in hexanes) to afford (9H-fluoren-9-yl)methyl (1-((2,2-diethoxyethyl)(4-methoxybenzyl)amino)-1-oxopropan-2-yl)carbamate (28 g, 51.22 mmol, 64.8% yield) as a gummy liquid. MS (ESI) m/z [M+H-EtOH]⁺: 501.2.

Step 3: Synthesis of 2-amino-N-(2,2-diethoxyethyl)-N-(4-methoxybenzyl)propanamide. To a solution of (9H-fluoren-9-yl) methyl (1-((2,2-diethoxyethyl)(4-methoxybenzyl)amino)-1-oxopropan-2-yl)carbamate (28 g, 51.22 mmol) in CH₂Cl₂ (30 mL) was added diethylamine (200 mL). The reaction mixture was stirred at room temperature for 3 h. After complete consumption of the starting material (monitored by TLC), the reaction mixture was concentrated and the crude obtained was purified by column chromatography (Silica 100-200 mesh; 5% MeOH in DCM) to afford 2-amino-N-(2,2-diethoxyethyl)-N-(4-methoxybenzyl)propanamide (14.5 g, 44.75 mmol, 87% yield) as a viscous liquid. MS (ESI) m/z [M+H-EtOH]*: 279.05.

Step 4: Synthesis of (9H-fluoren-9-yl)methyl (3-((1-((2,2-diethoxyethyl)(4-methoxybenzyl)amino)-1-oxopropan-2-yl)amino)-3-oxopropyl)carbamate. To a stirred solution of 3-((((9H-fluoren-9-yl)methoxy)carbonyl)amino)propanoic acid (14.78 g, 47.53 mmol) in dry DMF (120 mL) maintained at 0° C. was added HATU (18.06 g, 47.53 mmol), DIPEA (9.21 mL, 51.85 mmol) followed by 2-amino-N-(2,2-diethoxyethyl)-N-(4-methoxybenzyl)propanamide (14 g, 43.20 mmol). The reaction mixture was stirred for 16 h at room temperature. After completion, the reaction mixture was quenched with ice cold water (200 mL) and the aqueous layer was extracted with EtOAc (200 mL×2). The organic layer was washed with cold H₂O (500 mL) followed by saturated brine (200 mL), dried over Na₂SO₄ and concentrated under reduced pressure. The crude obtained was purified by column chromatography (Silica 100-200 mesh; 30% EtOAc in hexanes) to afford (9H-fluoren-9-yl)methyl (3-((1-((2,2-diethoxyethyl)(4-methoxybenzyl)amino)-1-oxopropan-2-yl)amino)-3-oxopropyl)carbamate (18 g, 29.14 mmol, 67.44% yield) as a viscous liquid. MS (ESI) m/z [M+H-EtOH]+: 572.

Step 5: Synthesis of (9H-fluoren-9-yl)methyl 8-(4-methoxybenzyl)-6-methyl-4,7-dioxohexahydro-2H-pyrazino[1,2-a]pyrimidine-1(6H)-carboxylate. A solution of (9H-fluoren-9-yl)methyl (3-((1-((2,2-diethoxyethyl)(4-methoxybenzyl)amino)-1-oxopropan-2-yl)amino)-3-oxopropyl)carbamate (18 g, 29.14 mmol) in formic acid (120 mL) was stirred at room temperature for 12 h. After completion, the reaction mixture was concentrated and the crude obtained was purified by column chromatography (Silica 100-200 mesh; 30% EtOAc in hexanes) to afford (9H-fluoren-9-yl)methyl 8-(4-methoxybenzyl)-6-methyl-4,7-dioxohexahydro-2H-pyrazino[1,2-a]pyrimidine-1(6H)-carboxylate (14.5 g, 27.58 mmol, 94% yield) as a solid. MS (ESI) m/z [M+H]⁺: 526.

Step 6: Synthesis of 8-(4-methoxybenzyl)-6-methylhexahydro-4H-pyrazino[1,2-a]pyrimidine-4,7(6H)-dione. To a solution of (9H-fluoren-9-yl)methyl 8-(4-methoxybenzyl)-6-methyl-4,7-dioxohexahydro-2H-pyrazino[1,2-a]pyrimidine-1(6H)-carboxylate (14 g, 26.63 mmol) in CH₂Cl₂ (150 mL) was added diethyl amine (100 mL) and the reaction mixture was stirred at room temperature for 3 h. After complete consumption of the starting material (monitored by TLC), the reaction mixture was concentrated and the crude obtained was purified by column chromatography (Silica 100-200 mesh; 5% MeOH in DCM) to afford 8-(4-methoxybenzyl)-6-methylhexahydro-4H-pyrazino[1,2-a]pyrimidine-4,7(6H)-dione (7 g, 23.07 mmol, 87% yield) as a sticky solid. MS (ESI) m/z [M+H]⁺: 304.

### Example S15. Synthesis of Intermediate Compound 8-(4-methoxybenzyl)-6-methylhexahydro-4H-pyrazino[1,2-a]pyrimidine-4,7(6H)-dione

The synthetic route for preparing this intermediate compound is shown in Scheme 15.

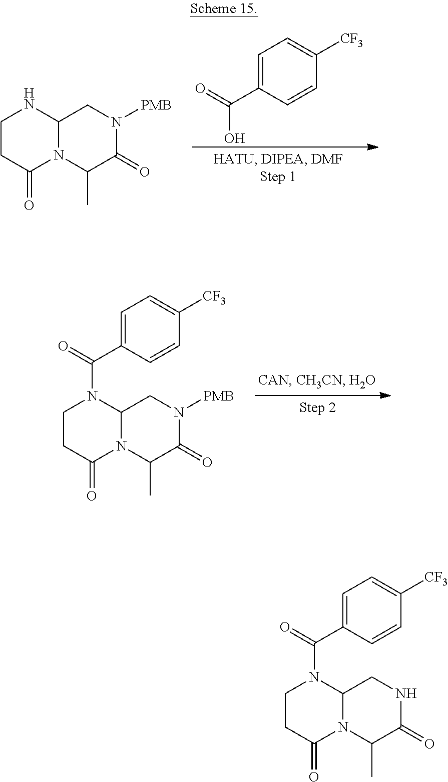

Step 1: Synthesis of 8-(4-methoxybenzyl)-6-methyl-1-(4-(trifluoromethyl)benzoyl)hexahydro-4H-pyrazino[1,2-a]pyrimidine-4,7(6H)-dione. To a solution of 4-(trifluoromethyl)benzoic acid (5.26 g, 27.69 mmol) in DMF (100 mL) maintained at 0° C. was added HATU (10.52 g, 27.69 mmol), DIPEA (12.30 mL, 69.23 mmol) followed by 8-(4-methoxybenzyl)-6-methylhexahydro-4H-pyrazino[1,2-a]pyrimidine-4,7(6H)-dione (7 g, 23.07 mmol), and the reaction mixture was stirred at room temperature for 12 h. After completion, the reaction mixture was quenched with ice cold water (200 mL) and the aqueous layer was extracted with EtOAc (200 mL×2). The organic layer was washed with cold H₂O (200 mL) followed by saturated brine (150 mL), dried over Na₂SO₄ and concentrated under reduced pressure. The crude obtained was purified by column chromatography (Silica 100-200 mesh; 30% EtOAc in hexanes) to afford 8-(4-methoxybenzyl)-6-methyl-1-(4-(trifluoromethyl)benzoyl)hexahydro-4H-pyrazino[1,2-a]pyrimidine-4,7(6H)-dione (9 g, 18.92 mmol, 82.04% yield) as a solid. MS (ESI) m/z [M+H]⁺: 476.15 and MS (ESI) m/z [M+Na]*: 498.05.

Step 2: Synthesis of 6-methyl-1-(4-(trifluoromethyl)benzoyl)hexahydro-4H-pyrazino[1,2-a]pyrimidine-4,7(6H)-dione. To a solution of 8-(4-methoxybenzyl)-6-methyl-1-(4-(trifluoromethyl)benzoyl)hexahydro-4H-pyrazino[1,2-a]pyrimidine-4,7(6H)-dione (9 g, 18.92 mmol) in CH₃CN:H₂O (2:1, 150 mL) maintained at 0° C., was added CAN (31.15 g, 56.82 mmol) and the reaction mixture was allowed to stir at room temperature for 3 h. Progress of the reaction was monitored by TLC. After completion, the reaction mixture was quenched with saturated solution of aq. NaHCO₃ (200 mL) and extracted with EtOAc (200 mL×2). Combined organic layer was washed with H₂O (200 mL) followed by saturated brine solution (150 mL), dried over Na₂SO₄ and concentrated under reduced pressure. The crude obtained was purified by column chromatography (Silica 100-200 mesh; 10% MeOH in DCM) to afford 6-methyl-1-(4-(trifluoromethyl)benzoyl)hexahydro-4H-pyrazino[1,2-a]pyrimidine-4,7(6H)-dione (3.5 g, 9.85 mmol, 52.8% yield) as a solid. MS (ESI) m/z [M+H+CH₃CN]⁺: 397.0. ¹H NMR (400 MHz, DMSO-d6) δ 1.25-1.46 (m, 3H) 2.15-2.30 (m, 1H) 2.56-2.69 (m, 1H) 3.16 (d, J=4.99 Hz, 1H) 3.22-3.30 (m, 1H) 3.42-3.72 (m, 2H) 4.70-4.87 (m, 1H) 5.85-5.95 (m, 1H) 7.75 (d, J=7.98 Hz, 2H) 7.86 (d, J=7.98 Hz, 2H) 8.11 (brs, 1H).

### Example S16. General Procedure A for the Synthesis of Final Compounds

To a solution of 6-methyl-1-(4-(trifluoromethyl)benzoyl)hexahydro-4H-pyrazino[1,2-a]pyrimidine-4,7(6H)-dione (200 mg, 0.56 mmol) in DMF (2 mL) was added KO^{t}Bu (1M in THF, 1.69 mmol, 1.69 mL) followed by alkyl halide (1.12 mmol), and the reaction mixture was exposed to microwave irradiation at 120° C. for 1 h. The reaction mixture was cooled to room temperature and quenched with H₂O (25 mL). The aqueous layer was extracted with EtOAc (10 mL×3). The combined organic layers were washed with brine and concentrated. The crude product obtained was purified by CombiFlash.

### Example S17. Synthesis of Compound 15

Compound 15 was synthesized by General Procedure A using (bromomethyl)cyclopentane as the alkyl halide. MS (ESI) m/z [M+H]⁺: 438.65. ¹H NMR (400 MHz, DMSO-d6) δ 1.02-1.26 (m, 3H) 1.28-1.42 (m, 2H) 1.44-1.76 (m, 6H) 1.80-2.08-2.33 (m, 2H) 2.55-2.71 (m, 1H) 3.22 (dd, J=12.96, 7.48 Hz, 1H) 3.26-3.32 (m, 1H) 3.39 (d, J=6.98 Hz, 1H) 3.49-3.57 (m, 1H) 3.59-3.74 (m, 1H) 3.76-3.91 (m, 1H) 4.80-4.90 (m, 1H) 5.95-6.05 (m, 1H) 7.72-7.79 (m, 2H) 7.84-7.91 (m, 2H).

### Example S18. Synthesis of Compound 16

Compound 16 was synthesized by General Procedure A using bromomethylcyclobutane as the alkyl halide. MS (ESI) m/z [M+H]⁺: 424.15. ¹H NMR (400 MHz, DMSO-d6) δ 1.29-1.44 (m, 2H) 1.58-1.89 (m, 4H) 1.90-2.08 (m, 2H) 2.16-2.31 (m, 1H) 2.55-2.70 (m, 2H) 3.18-3.31 (m, 1H) 3.25-3.26 (m, 1H) 3.34-3.42 (m, 1H) 3.36-3.57 (m, 2H) 3.60-3.69 (m, 1H) 3.71-3.83 (m, 1H) 4.75-4.89 (m, 1H) 5.90-6.05 (m, 1H) 7.70-7.79 (m, 2H) 7.87 (d, J=8.31 Hz, 2H).

### Example S19. Synthesis of Compound 19

Compound 19 was synthesized by General Procedure A using (2-bromoethyl)cyclopentane as the alkyl halide. MS (ESI) m/z [M+H]⁺: 452.35. ¹H NMR (400 MHz, DMSO-d6) δ 0.94-1.18 (m, 3H) 1.26-1.61 (m, 9H) 1.66-1.83 (m, 2H) 2.16-2.31 (m, 1H) 2.56-2.70 (m, 1H) 3.16-3.28 (m, 1H) 3.35-3.56 (m, 3H) 3.60-3.73 (m, 1H) 3.77-3.90 (m, 1H) 4.72-4.92 (m, 1H) 5.94-6.06 (m, 1H) 7.77 (d, J=7.98 Hz, 2H) 7.87 (d, J=7.98 Hz, 2H).

### Example S20. Synthesis of Compound 20

Compound 20 was synthesized by General Procedure A using (2-bromoethyl)cyclobutane as the alkyl halide. MS (ESI) m/z [M+H]⁺: 438.25. ¹H NMR (400 MHz, DMSO-d6) δ 1.27-1.44 (m, 3H) 1.50-1.71 (m, 4H) 1.71-1.88 (m, 2H) 1.93-2.09 (m, 2H) 2.13-2.34 (m, 2H) 2.56-2.70 (m, 2H) 3.25-3.32 (m, 1H) 3.35-3.42 (m, 1H) 3.45-3.55 (m, 1H) 3.59-3.72 (m, 1H) 3.74-3.90 (m, 1H) 4.75-4.89 (m, 1H) 5.94-6.05 (m, 1H) 7.71-7.79 (m, 2H) 7.87 (d, J=8.31 Hz, 2H).

### Example S21. Synthesis of Compound 21

Compound 21 was synthesized by General Procedure A using 1-bromobutane as the alkyl halide. MS (ESI) m/z [M+H]⁺: 412.20. ¹H NMR (400 MHz, DMSO-d6) δ 0.81-0.97 (m, 3H) 1.15-1.57 (m, 7H) 2.15-2.31 (m, 1H) 2.57-2.69 (m, 1H) 3.14-3.28 (m, 1H) 3.35-3.60 (m, 3H) 3.62-3.73 (m, 1H) 3.74-3.92 (m, 1H) 4.75-4.91 (m, 1H) 5.94-6.06 (m, 1H) 7.76 (d, J=7.34 Hz, 2H) 7.87 (d, J=7.83 Hz, 2H).

### Example S22. Synthesis of Compound 22

Compound 22 was synthesized by General Procedure A using 4-bromobut-1-ene as the alkyl halide. MS (ESI) m/z [M+H]⁺: 410.20. ¹H NMR (400 MHz, DMSO-d6) δ 1.28-1.45 (m, 3H) 2.14-2.38 (m, 3H) 2.55-2.69 (m, 1H) 3.36-3.57 (m, 4H) 3.58-3.72 (m, 1H) 3.75-3.89 (m, 1H) 4.75-4.90 (m, 1H) 4.98-5.19 (m, 2H) 5.69-5.84 (m, 1H) 5.93-6.05 (m, 1H) 7.76 (d, J=7.98 Hz, 2H) 7.88 (d, J=7.98 Hz, 2H).

### Example S23. Synthesis of Compound 23

Compound 23 was synthesized by General Procedure A using 1-bromo-2-methylpropane as the alkyl halide. MS (ESI) m/z [M+H]⁺: 412.25. ¹H NMR (400 MHz, DMSO-d6) δ 0.80-0.96 (m, 6H) 1.30-1.48 (m, 3H) 1.85-2.03 (m, 1H) 2.15-2.31 (m, 1H) 2.57-2.70 (m, 1H) 3.06-3.16 (m, 1H) 3.18-3.28 (m, 1H) 3.36-3.45 (m, 1H) 3.44-3.57 (m, 1H) 3.60-3.74 (m, 1H) 3.73-3.87 (m, 1H) 4.77-4.92 (m, 1H) 5.93-6.07 (m, 1H) 7.76 (d, J=7.48 Hz, 2H) 7.87 (d, J=7.48 Hz, 2H).

### Example S24. Synthesis of Compound 24

Compound 24 was synthesized by General Procedure A using 2-bromopropane as the alkyl halide. MS (ESI) m/z [M+H]⁺: 398.55. ¹H NMR (400 MHz, DMSO-d6) δ 1.10 (d, J=5.49 Hz, 6H) 1.28-1.45 (m, 3H) 2.16-2.24 (m, 1H) 2.56-2.71 (m, 1H) 3.34-3.40 (m, 1H) 3.44-3.79 (m, 3H) 4.59-4.72 (m, 1H) 4.75-4.90 (m, 1H) 5.86-6.00 (m, 1H) 7.79 (d, J=7.98 Hz, 2H) 7.83-7.92 (m, 2H).

### Example S25. Synthesis of Intermediate Compound 1-(4-(difluoromethoxy)benzoyl)-6-methylhexahydro-4H-pyrazino[1,2-a]pyrimidine-4,7(6H)-dione

The synthetic route for preparing this intermediate compound is shown in Scheme 16.

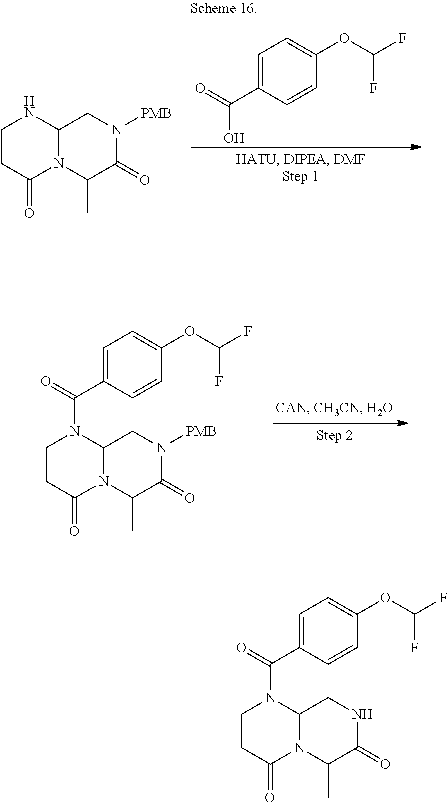

Step 1: Synthesis of 1-(4-(difluoromethoxy)benzoyl)-8-(4-methoxybenzyl)-6-methylhexahydro-4H-pyrazino[1,2-a]pyrimidine-4,7(6H)-dione. To a solution of 4-(difluoromethoxy)benzoic acid (1.71 g, 9.08 mmol) in DMF (25 mL) maintained at 0° C. was added HATU (3.45 g, 9.08 mmol), DIPEA (4.34 mL, 24.8 mmol) followed by 8-(4-methoxybenzyl)-6-methylhexahydro-4H-pyrazino[1,2-a]pyrimidine-4,7(6H)-dione (2.5 g, 8.25 mmol) and reaction mixture was stirred at room temperature for 12 h. After completion, the reaction mixture was quenched with ice cold water (50 mL) and the aqueous layer was extracted with EtOAc (100 mL×2). The organic layer was washed with cold H₂O (100 mL) followed by saturated brine (100 mL), dried over Na₂SO₄ and concentrated under reduced pressure. The crude obtained was purified by column chromatography (Silica 100-200 mesh; 30% EtOAc in hexanes) to afford 1-(4-(difluoromethoxy)benzoyl)-8-(4-methoxybenzyl)-6-methylhexahydro-4H-pyrazino[1,2-a]pyrimidine-4,7(6H)-dione (3.5 g, 7.38 mmol, 89.5% yield) as a solid. MS (ESI) m/z [M+H]⁺: 474.12.

Step 2: Synthesis of 1-(4-(difluoromethoxy)benzoyl)-6-methylhexahydro-4H-pyrazino[1,2-a]pyrimidine-4,7(6H)-dione. To a solution of 1-(4-(difluoromethoxy)benzoyl)-8-(4-methoxybenzyl)-6-methylhexahydro-4H-pyrazino[1,2-a]pyrimidine-4,7(6H)-dione (3.0 g, 6.34 mmol) in CH₃CN:H₂O (2:1, 45 mL) maintained at 0° C., was added CAN (12.0 g, 21.90 mmol) and the reaction mixture was allowed to stir at room temperature for 3 h. Progress of the reaction was monitored by TLC. After completion, the reaction mixture was quenched with saturated solution of aq. NaHCO₃ (100 mL) and extracted with EtOAc (200 mL×2). The combined organic layer was washed with H₂O (250 mL) followed by saturated brine solution (250 mL), dried over Na₂SO₄ and concentrated under reduced pressure. The crude obtained was purified by column chromatography (Silica 100-200 mesh; 10% MeOH in DCM) to afford 1-(4-(difluoromethoxy)benzoyl)-6-methylhexahydro-4H-pyrazino[1,2-a]pyrimidine-4,7(6H)-dione (2.0 g, 5.66 mmol, 89.6% yield) as a solid. MS (ESI) m/z [M+H]⁺: 353.95. ¹H NMR (400 MHz, DMSO-d6) δ 1.10-1.39 (m, 3H) 2.17-2.18 (m, 1H) 2.52-2.68 (m, 1H) 3.18-3.27 (m, 2H) 3.44-3.71 (m, 2H) 4.69-4.83 (m, 1H) 5.75-5.92 (m, 1H) 7.24 (d, J=7.83 Hz, 2H) 7.32 (t, J=72.0 Hz, 1H) 7.57 (d, J=8.31 Hz, 2H) 8.04 (brs, 1H).

### Example S26. General Procedure B for the Synthesis of Final Compounds

To a solution of 1-(4-(difluoromethoxy)benzoyl)-6-methylhexahydro-4H-pyrazino[1,2-a]pyrimidine-4,7(6H)-dione (200 mg, 0.56 mmol) in DMF (4 mL) maintained at 0° C. was added NaH (122 mg, 2.8 mmol, 55% dispersion in mineral oil) and the reaction mixture was stirred at the same temperature for 15 minutes. To this reaction mixture was added alkyl halide (1.6 mmol) and the reaction mixture was allowed to warm to room temperature and stirred for 3 h. After completion, the reaction mixture was quenched with ice cold H₂O (15 mL) and aqueous layer was extracted with EtOAc (15 mL×3). The combined organic layer was washed with brine and concentrated. The crude product obtained was purified by CombiFlash.

### Example S27. Synthesis of Compound 13

Compound 13 was synthesized by General Procedure B using (bromomethyl)cyclopentane as the alkyl halide. MS (ESI) m/z [M+H]⁺: 436.05. ¹H NMR (400 MHz, DMSO-d6) δ 1.07-1.16 (m, 3H) 1.32 (d, J=6.48 Hz, 3H) 1.41-1.73 (m, 7H) 2.06-2.21 (m, 1H) 2.21-2.34 (m, 1H) 2.54-2.70 (m, 1H) 3.14-3.29 (m, 1H) 3.35-3.45 (m, 1H) 3.52-3.69 (m, 1H) 3.75-3.93 (m, 1H) 4.75-4.91 (m, 1H) 5.88-5.99 (m, 1H) 7.27 (d, J=8.48 Hz, 2H) 7.35 (t, J=72.0 Hz, 1H) 7.61 (d, J=8.98 Hz, 2H).

### Example S28. Synthesis of Compound 14

Compound 14 was synthesized by General Procedure B using (bromomethyl)cyclobutane as the alkyl halide. MS (ESI) m/z [M+H]⁺: 421.14. ¹H NMR (400 MHz, DMSO-d6) δ 1.16-1.25 (m, 1H) 1.27-1.43 (m, 3H) 1.54-1.73 (m, 2H) 1.73-1.86 (m, 2H) 1.89-2.03 (m, 2H) 2.24 (d, J=17.12 Hz, 1H) 2.53-2.69 (m, 2H) 3.20-3.28 (m, 1H) 3.29-3.40 (m, 1H) 3.40-3.66 (m, 2H) 3.69-3.87 (m, 1H) 4.75-4.86 (m, 1H) 5.74-6.02 (m, 1H) 7.26 (d, J=8.31 Hz, 2H)) 7.33 (t, J=72.0 Hz, 1H) 7.59 (d, J=8.31 Hz, 2H).

### Example S29. Synthesis of Compound 17

Compound 20 was synthesized by General Procedure B using 1-bromobutane as the alkyl halide. MS (ESI) m/z [M+H]⁺: 410.0. ¹H NMR (400 MHz, DMSO-d6) δ 0.81-0.96 (m, 3H) 1.15-1.39 (m, 4H) 1.40-1.55 (m, 2H) 2.26 (d, J=16.95 Hz, 1H) 2.53-2.70 (m, 2H) 3.12-3.30 (m, 2H) 3.38-3.46 (m, 1H) 3.56-3.74 (m, 2H) 3.75-3.92 (m, 1H) 4.84 (q, J=6.81 Hz, 1H) 5.86-6.06 (m, 1H) 7.28 (d, J=7.98 Hz, 2H) 7.36 (t, J=72.0 Hz, 1H) 7.62 (d, J=8.48 Hz, 2H).

### Example S30. Synthesis of Compound 18

Compound 18 was synthesized by General Procedure B using 4-bromobut-1-ene as the alkyl halide. MS (ESI) m/z [M+H]⁺: 408.06. ¹H NMR (400 MHz, DMSO-d6) δ 1.16-1.45 (m, 3H) 2.18-2.33 (m, 3H) 2.53-2.70 (m, 1H) 3.36-3.46 (m, 3H) 3.51-3.72 (m, 2H) 3.74-3.90 (m, 1H) 4.84 (q, J=6.65 Hz, 1H) 4.91-5.15 (m, 2H) 5.67-5.84 (m, 1H) 5.86-6.03 (m, 1H) 7.29 (d, J=8.48 Hz, 2H) 7.36 (t, J=72.0 Hz, 1H) 7.61 (d, J=8.48 Hz, 2H).

### Example S31. Synthesis of Compound 27

Compound 27 was synthesized by General Procedure B using 2-(bromomethyl)tetrahydrofuran as the alkyl halide. MS (ESI) m/z [M+H]⁺: 438.1. ¹H NMR (400 MHz, CDCl₃) δ 7.48-7.55 (m, 2H), 7.20-7.30 (m, 2H), 6.40-6.76 (m, 1H), 5.90-6.20 (m, 1H), 5.16-5.26 (m, 1H), 4.06-4.17 (m, 2H), 3.82-3.92 (m, 4H), 3.61-3.77 (m, 2H), 2.83-2.99 (m, 1H), 2.47-2.59 (m, 2H), 2.01-2.12 (m, 4H), 1.49 (s, 3H).

### Example S32. Synthesis of Compound 28

Compound 28 was synthesized by General Procedure B using (2-bromoethyl)benzene as the alkyl halide. MS (ESI) m/z [M+H]⁺: 458.10. ¹H NMR (400 MHz, CDCl₃) δ 7.40-7.50 (m, 2H), 7.20-7.28 (m, 2H), 7.33-7.43 (m, 5H), 6.40-6.76 (m, 1H), 5.90-6.20 (m, 1H), 5.16-5.26 (m, 1H), 3.72-3.96 (m, 2H), 3.44-3.52 (m, 1H), 3.25-3.35 (m, 2H), 2.83-2.99 (m, 2H), 2.47-2.59 (m, 1H), 2.42-2.60 (m, 1H), 2.30-2.57 (m, 1H), 1.49 (s, 3H).

### Example S33. Synthesis of Compound 29

Compound 29 was synthesized by General Procedure B using 4-(2-bromoethyl)pyridine as the alkyl halide. MS (ESI) m/z [M+H]⁺: 459.10. ¹H NMR (400 MHz, CDCl₃) δ 8.50-8.58 (m, 2H), 7.24-7.46 (m, 4H), 7.18 (d, J=7.99 Hz, 2H), 6.40-6.76 (m, 1H), 5.90-6.20 (m, 1H), 5.16-5.26 (m, 1H), 3.72-3.96 (m, 2H), 3.44-3.52 (m, 1H), 3.25-3.35 (m, 2H), 2.83-2.99 (m, 2H), 2.47-2.59 (m, 1H), 2.42-2.60 (m, 1H), 2.30-2.57 (m, 1H), 1.49 (s, 3H).

### Example S34. Synthesis of Compound 30

Compound 30 was synthesized by General Procedure B using (3-bromopropyl)cyclopropane as the alkyl halide. MS (ESI) m/z [M+H]⁺: 459.10. ¹H NMR (400 MHz, CDCl₃) δ 8.50-8.58 (m, 2H), 7.24-7.46 (m, 4H), 7.18 (d, J=7.99 Hz, 2H), 6.40-6.76 (m, 1H), 5.90-6.20 (m, 1H), 5.16-5.26 (m, 1H), 3.72-3.96 (m, 2H), 3.44-3.52 (m, 1H), 3.25-3.35 (m, 2H), 2.83-2.99 (m, 2H), 2.47-2.59 (m, 1H), 2.42-2.60 (m, 1H), 2.30-2.57 (m, 1H), 1.49 (s, 3H).

### Example S35. Synthesis of Compound 31

Compound 31 was synthesized by General Procedure B using (2-bromoethyl)cyclopropane as the alkyl halide. MS (ESI) m/z [M+H]⁺: 422.2. ¹H NMR (400 MHz, CDCl₃) δ 7.48 (d, J=8.01 Hz, 2H), 7.20-7.28 (m, 2H), 6.40-6.76 (m, 1H), 5.90-6.20 (m, 1H), 5.16-5.26 (m, 1H), 3.72-3.96 (m, 1H), 3.46-3.64 (m, 5H), 2.46-2.64 (m, 2H), 1.43-1.56 (m, 5H), 0.43-0.65 (m, 2H), 0.75-0.85 (m, 2H).

### Example S36. Synthesis of Compound 32

Compound 32 was synthesized by General Procedure B using 1-bromo-2-methoxyethane as the alkyl halide. MS (ESI) m/z [M+H]⁺: 412.1. ¹H NMR (400 MHz, DMSO-d6) δ 7.52-7.62 (m, 2H), 7.16-7.34 (m, 3H), 5.85-5.95 (m, 1H), 4.80-4.90 (m, 1H), 3.85-3.95 (m, 1H), 3.70-3.80 (m, 2H), 3.25-3.46 (m, 5H), 3.22 (s, 3H), 2.62-2.72 (m, 1H), 2.20-2.30 (m, 1H), 1.49 (s, 3H).

### Example S37. Synthesis of Compound 33

Compound 33 was synthesized by General Procedure B using 1-bromo-3-methoxypropane as the alkyl halide. MS (ESI) m/z [M+H]⁺: 426.20. ¹H NMR (400 MHz, DMSO-d6) δ 7.52-7.62 (m, 2H), 7.16-7.34 (m, 3H), 5.85-5.95 (m, 1H), 4.80-4.90 (m, 1H), 3.85-3.95 (m, 1H), 3.70-3.80 (m, 2H), 3.58-3.68 (m, 2H), 3.45-3.55 (m, 4H), 3.22 (s, 3H), 2.62-2.72 (m, 1H), 2.20-2.30 (m, 2H), 1.49 (s, 3H).

### Example S38. Synthesis of Compound 36

Compound 36 was synthesized by General Procedure B using (2-bromoethyl)methylsulfone as the alkyl halide. MS (ESI) m/z [M+H]⁺: 459.95. ¹H NMR (400 MHz, CHLOROFORM) δ 7.49 (d, J=8.01 Hz, 2H), 7.15-7.26 (m, 2H), 6.40-6.76 (m, 1H), 5.90-6.20 (m, 1H), 5.15-5.25 (m, 1H), 3.86-3.97 (m, 3H), 3.66-3.77 (m, 2H), 3.38-3.49 (m, 3H), 2.97 (s, 3H), 2.59-2.69 (m, 2H), 1.49 (s, 3H).

### Example S39. Synthesis of Compound 34

Step 1. To a solution of 1-(4-(difluoromethoxy)benzoyl)-6-methylhexahydro-4H-pyrazino[1,2-a]pyrimidine-4,7(6H)-dione (0.300 g, 0.849 mmol) in DMF (6 mL) was added Cs₂CO₃ (0.827 g, 2.547 mmol) followed by (2-bromoethoxy)(tert-butyl)dimethylsilane (0.243 g, 1.018 mmol) at 0° C. and the reaction mixture was heated at 120° C. in sealed tube for 1 h. Progress of the reaction was monitored by TLC. After completion, the reaction mixture was slowly quenched with ice cold water (30 mL) and extracted with EtOAc (50 mL). Combined organic layer was washed with ice cold brine solution (3×30 mL), dried over Na₂SO₄ and concentrated under reduced pressure to afford 8-(2-((tert-butyldimethylsilyl)oxy)ethyl)-1-(4-(difluoromethoxy)benzoyl)-6-methylhexahydro-4H-pyrazino[1,2-a]pyrimidine-4,7(6H)-dione (0.250 g, crude). The crude compound was as such used for next reaction without carried out further purification. MS (ESI) m/z [M+H]⁺: 512.10.

Step 2. To a solution of 8-(2-((tert-butyldimethylsilyl)oxy)ethyl)-1-(4-(difluoromethoxy)benzoyl)-6-methylhexahydro-4H-pyrazino[1,2-a]pyrimidine-4,7(6H)-dione (0.250 g, 0.4886 mmol) in THE (5 mL) was added TBAF (3 mL) 0° C. temperature. The reaction mixture was allowed to attain room temperature and stirred for 6 h. Progress of the reaction was monitored by TLC. After completion, the reaction mixture was slowly quenched with ice cold water (5 mL) and extracted with EtOAc (2×10 mL). Combined organic layer was washed with ice cold brine solution (10 mL), dried over Na₂SO₄ and concentrated under reduced pressure to get crude compound. The crude compound obtained was purified by column chromatography (Silicagel 60-120 mesh; 10% MeOH in DCM) to afford 1-(4-(difluoromethoxy)benzoyl)-8-(2-hydroxyethyl)-6-methylhexahydro-4H-pyrazino[1,2-a]pyrimidine-4,7(6H)-dione (0.102 g, 52% yield) white solid. MS (ESI) m/z [M+H]⁺: 398.2. ¹H NMR (400 MHz, DMSO-d6) δ 7.52-7.62 (m, 2H), 7.16-7.34 (m, 3H), 5.92-6.02 (m, 1H), 6.78-6.88 (m, 2H), 3.86-3.92 (m, 1H), 3.47-3.62 (m, 6H), 3.21-3.31 (m, 1H), 2.57-2.67 (m, 1H), 2.25-3.35 (m, 1H), 1.49 (s, 3H).

### Example S40. Synthesis of Compound 35

Step 1. To a solution of 1-(4-(difluoromethoxy)benzoyl)-6-methylhexahydro-4H-pyrazino[1,2-a]pyrimidine-4,7(6H)-dione (0.300 g, 0.849 mmol) in DMF (6 mL) was added NaH (0.050 g, 1.274 mmol) followed by 2-bromoacetonitrile (0.112 g, 0.933 mmol) at 0° C. and the reaction mixture was allowed to stand for room temperature for 1 h. Progress of the reaction was monitored by TLC. After completion, the reaction mixture was slowly quenched with ice cold water (70 mL) and extracted with EtOAc (100 mL). Combined organic layer was washed with ice cold brine solution (100 mL), dried over Na₂SO₄ and concentrated under reduced pressure to get crude compound. The crude compound obtained was purified by column chromatography (Silicagel 60-120 mesh; 10% MeOH in DCM) to afford 2-(1-(4-(difluoromethoxy)benzoyl)-6-methyl-4,7-dioxooctahydro-8H-pyrazino[1,2-a]pyrimidin-8-yl)acetonitrile (0.120 g, 36% yield) white solid. MS (ESI) m/z [M+H]⁺: 393.05.

Step 2. To a solution of 2-(1-(4-(difluoromethoxy)benzoyl)-6-methyl-4,7-dioxooctahydro-8H-pyrazino[1,2-a]pyrimidin-8-yl)acetonitrile (0.120 g, 0.305 mmol) in ethanol (5 mL) was added Conc. HCl (0.100 mL) followed by Platinum oxide (0.012 g, 0.030 mmol) at room temperature and the reaction mixture was heated under Hydrogen gas atmosphere for 3 h. Progress of the reaction was monitored by TLC. After completion, the reaction mixture was filtered through a pad of Celite. The Celite pad was washed with ethanol (20 mL) and filtrate was concentrated under reduced pressure to get crude compound. The crude compound was triturated with n pentane to afford 8-(2-aminoethyl)-1-(4-(difluoromethoxy)benzoyl)-6-methylhexahydro-4H-pyrazino[1,2-a]pyrimidine-4,7(6H)-dione (0.110 g, 90%) yield) white solid. MS (ESI) m/z [M+H]⁺: 397.05. ¹H NMR (400 MHz, DMSO d6) δ 7.96 (s, 2H), 7.55-7.65 (m, 2H), 7.20-7.35 (m, 3H), 5.90-6.20 (m, 1H), 4.85-4.95 (m, 1H), 3.82-3.92 (m, 1H), 3.55-3.85 (m, 2H), 3.35-3.45 (m, 3H), 2.95-3.05 (m, 2H), 2.60-2.70 (m, 1H), 2.20-2.30 (m, 1H), 1.35 (s, 3H).

### Example S41. General Procedure C for the Synthesis of Final Compounds

To a solution of 1-(4-(difluoromethoxy)benzoyl)-6-methylhexahydro-4H-pyrazino[1,2-a]pyrimidine-4,7(6H)-dione (0.200 g, 0.566 mmol) in DMF (5 mL) was added Cs₂CO₃ (0.735 g, 2.264 mmol, 4 eq) followed by alkyl halide (0.679 mmol, 1.2 eq) at 0° C. and the reaction mixture was heated at 50° C. under microwave irradiation for 1 h. Progress of the reaction was monitored by TLC. After completion, the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to get crude compound. The crude compound obtained was purified by column chromatography to provide the final compound.

### Example S42. Synthesis of Compound 25

Compound 25 was synthesized by General Procedure C using 2-(2-iodoethyl)furan as the alkyl halide. MS (ESI) m/z [M+H]⁺: 448.10. ¹H NMR: δ 7.40-7.50 (m, 2H), 7.28-7.38 (m, 1H), 7.15-7.25 (m, 2H), 6.39-6.78 (m, 1H), 6.25-6.35 (m, 1H), 5.90-6.12 (m, 2H), 5.25-5.35 (m, 1H), 5.10-5.20 (m, 1H), 3.70-3.80 (m, 1H), 3.50-3.60 (m, 1H), 3.20-3.40 (m, 2H), 2.95-3.05 (m, 3H), 2.45-2.60 (m, 2H), 1.59 (s, 3H).

### Example S43. Synthesis of Compound 26

Compound 26 was synthesized by General Procedure C using 2-(2-bromoethyl)thiophene as the alkyl halide. MS (ESI) m/z [M+H]⁺: 464.1. ¹H NMR (400 MHz, CDCl₃) δ 7.40-7.48 (m, 2H), 7.15-7.26 (m, 3H), 6.85-6.95 (m, 2H), 6.39-6.95 (m, 2H), 5.90-6.20 (m, 1H), 5.15-5.25 (m, 1H), 3.72-3.96 (m, 2H), 3.47-3.54 (m, 1H), 3.32-3.42 (m, 3H), 3.10-3.20 (m, 2H), 2.42-2.56 (m, 2H), 1.49 (s, 3H).

### Example S44. Synthesis of Intermediate Compound 1-(4-(difluoromethoxy)benzyl)-6-methylhexahydro-4H-pyrazino[1,2-a]pyrimidine-4,7(6H)-dione

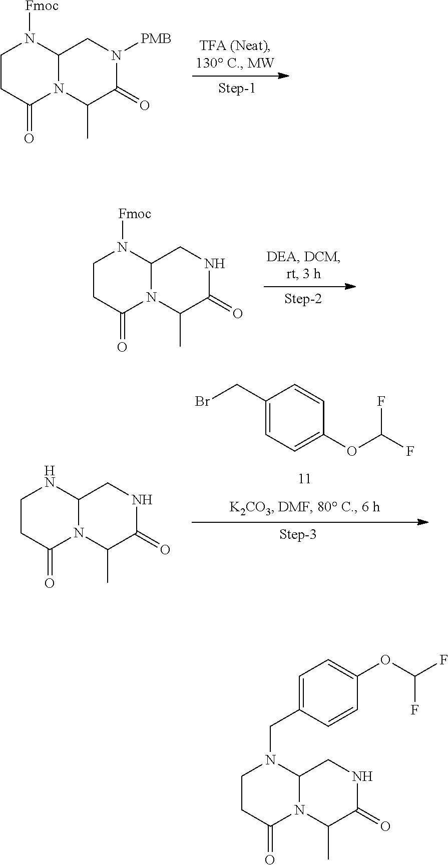

Step 1: Synthesis of (9H-fluoren-9-yl)methyl 6-methyl-4,7-dioxohexahydro-2H-pyrazino[1,2-a]pyrimidine-1(6H)-carboxylate. A solution of (9H-fluoren-9-yl)methyl 8-(4-methoxybenzyl)-6-methyl-4,7-dioxohexahydro-2H-pyrazino[1,2-a]pyrimidine-1(6H)-carboxylate (1.0 g, 26.63 mmol) in TFA (10 mL) was stirred at 130° C. for 2 h in microwave. After complete consumption of the starting material (monitored by TLC), the reaction mixture was concentrated under vacuum and the crude product was extracted with ethylacetate (100 ml) and saturated solution of sodium bicarbonate. The organic layer was dried over anhydrous Na₂SO₄ and concentrated under vacuum, and purified by column chromatography (Silica 100-200 mesh; 5% MeOH in DCM) to afford (9H-fluoren-9-yl)methyl 6-methyl-4,7-dioxohexahydro-2H-pyrazino[1,2-a]pyrimidine-1(6H)-carboxylate (300 mg, 42% yield) as a sticky solid. MS (ESI) m/z [M+H]⁺: 406.

Step 2: Synthesis of 6-methylhexahydro-4H-pyrazino[1,2-a]pyrimidine-4,7(6H)-dione. To a solution of (9H-fluoren-9-yl)methyl 6-methyl-4,7-dioxohexahydro-2H-pyrazino[1,2-a]pyrimidine-1(6H)-carboxylate (300 mg, 0.74 mmol) in CH₂C₂ (5 mL) was added diethylamine (6 mL). The reaction mixture was stirred at room temperature for 3 h. After complete consumption of the starting material (monitored by TLC), the reaction mixture was concentrated and the crude product was purified by column chromatography (Silica 100-200 mesh; 10% MeOH in DCM) to afford 6-methylhexahydro-4H-pyrazino[1,2-a]pyrimidine-4,7(6H)-dione (120 mg, 92% yield) as a white solid. MS (ESI) m/z [M+H]⁺: 184.

Step 3: Synthesis of 1-(4-(difluoromethoxy)benzyl)-6-methylhexahydro-4H-pyrazino[1,2-a]pyrimidine-4,7(6H)-dione. To a solution of 6-methylhexahydro-4H-pyrazino[1,2-a]pyrimidine-4,7(6H)-dione (0.700 g, 3.820 mmol) in DMF (8.0 mL) was added K₂CO₃ (1.58 g, 11.46 mmol) at room temperature and stirred for 10 min. To the resulting reaction mixture was added 1-(bromomethyl)-4-(difluoromethoxy)benzene (1.086 g, 4.584 mmol) and the reaction mixture was heated at 80° C. for 6 h. Progress of the reaction was monitored by TLC. After completion, the reaction mixture was cooled to room temperature, quenched with water (50 mL) and extracted with EtOAc (50 mL×2). The combined organic layers were washed with saturated brine solution (20 mL), dried over Na₂SO₄ and concentrated under reduced pressure. The crude product was purified by column chromatography (Silica 100-200 mesh; 5% MeOH in DCM) to afford 1-(4-(difluoromethoxy)benzyl)-6-methylhexahydro-4H-pyrazino[1,2-a]pyrimidine-4,7(6H)-dione (0.550 g, 43.0% yield) as an off-white solid. MS (ESI) m/z [M+H]⁺: 340.34.

### Example S45. General Procedure D for Synthesis of Final Compounds

To a solution of 1-(4-(difluoromethoxy)benzyl)-6-methylhexahydro-4H-pyrazino[1,2-a]pyrimidine-4,7(6H)-dione (0.100 g, 0.2949 mmol) in DMF (2 mL) was added NaH (0.021 g, 0.8847 mmol) at 0° C. followed by the appropriate alkyl halide (2 eq.) and the reaction mixture was allowed to warm to room temperature and stirred for 5 h. Progress of the reaction was monitored by TLC. After completion, the reaction mixture was slowly quenched with saturated solution of aq. NaHCO₃ (2 mL) and extracted with EtOAc (10 mL×2). The combined organic layers were washed with H₂O (5 mL) followed by saturated brine solution (5 mL), dried over Na₂SO₄ and concentrated under reduced pressure. The crude material was purified by combiflash column chromatography (5% MeOH in DCM) to afford the final product.

### Example S46. Synthesis of Compound 37

Compound 37 was synthesized by General Procedure D using 4-bromo-1,1,1-trifluorobutane as the alkyl halide. MS (ESI) m/z [M+H]⁺: 354.2. ¹H NMR (400 MHz, CDCl₃): δ 1.41 (d, J=7.13 Hz, 3H), 1.71-1.86 (m, 2H), 2.01-2.15 (m, 2H), 2.26-2.35 (m, 1H), 2.60-2.67 (m, 1H), 2.89-3.01 (m, 1H), 3.07-3.15 (m, 1H), 3.21-3.34 (m, 2H), 3.46-3.65 (m, 2H), 3.81-3.95 (m, 2H), 4.35-4.41 (m, 1H), 5.20-5.29 (m, 1H), 6.53 (t, J=72.0 Hz, 1H), 7.08-7.16 (m, 2H), 7.30-7.36 (m, 2H).

### Example S47. Synthesis of Compound 38

Compound 38 was synthesized by General Procedure D using (2-bromoethyl)cyclopentane as the alkyl halide. MS (ESI) m/z [M+H]⁺: 436.2. ¹H NMR (400 MHz, CDCl₃) δ 1.01-1.15 (m, 2H), 1.41 (d, J=7.13 Hz, 3H), 1.45-1.62 (m, 8H), 1.66-1.80 (m, 2H), 2.23-2.34 (m, 1H), 2.58-2.72 (m, 1H), 2.89-2.98 (m, 1H), 3.04-3.18 (m, 2H), 3.23-3.33 (m, 1H), 3.43-3.54 (m, 1H), 3.55-3.65 (m, 1H), 3.78-3.93 (m, 1H), 4.31-4.39 (m, 1H), 5.15-5.26 (m, 1H), 6.53 (t, J=72.0 Hz, 1H), 7.13 (d, J=8.50 Hz, 2H), 7.34 (d, J=8.50 Hz, 2H).

### Example S48. Synthesis of Compound 39

Compound 39 was synthesized by General Procedure D using 4-bromobut-1-ene as the alkyl halide. MS (ESI) m/z [M+H]⁺: 394.2. ¹H NMR (400 MHz, CDCl₃) δ 1.41 (d, J=7.13 Hz, 3H), 2.23-2.35 (m, 3H), 2.60-2.71 (m, 1H), 2.92-3.01 (m, 1H), 3.06-3.14 (m, 1H), 3.22-3.46 (m, 3H), 3.53-3.64 (m, 1H), 3.79-3.93 (m, 2H), 4.28-4.38 (m, 1H), 4.91-5.00 (m, 2H), 5.16-5.26 (m, 1H), 5.64-5.76 (m, 1H), 6.52 (t, J=72.0 Hz, 1H), 7.10-7.16 (m, 2H), 7.30-7.36 (m, 2H).

### Example S49. Synthesis of Compound 40

Compound 40 was synthesized by General Procedure D using (2-bromoethyl)cyclobutene as the alkyl halide. MS (ESI) m/z [M+H]⁺: 422.25 ¹H NMR (400 MHz, CDCl₃) δ 1.41 (d, J=7.13 Hz, 3H), 1.56-1.65 (m, 4H), 1.73-1.92 (m, 2H), 1.95-2.07 (m, 2H), 2.14-2.25 (m, 1H), 2.26-2.35 (m, 1H), 2.59-2.72 (m, 1H), 2.91-2.99 (m, 1H), 3.04-3.14 (m, 2H), 3.23-3.43 (m, 2H), 3.53-3.63 (m, 1H), 3.87 (q, J=13.38 Hz, 2H), 4.29-4.39 (m, 1H), 5.17-5.24 (m, 1H), 6.53 (t, J=72.0 Hz, 1H), 7.13 (d, J=8.63 Hz, 2H), 7.30-7.37 (m, 2H).

### Example S50. Synthesis of Compound 41

Compound 41 was synthesized by General Procedure D using 1-bromobutane as the alkyl halide. MS (ESI) m/z [M+H]⁺: 396.05. ¹H NMR (400 MHz, DMSO-d6) δ 0.86 (t, J=7.34 Hz, 3H), 1.14-1.24 (m, 2H), 1.24-1.30 (m, 2H), 1.38-1.50 (m, 2H), 1.98-2.10 (m, 1H), 2.53-2.61 (m, 2H), 2.64-2.77 (m, 2H), 3.07-3.25 (m, 3H), 3.32-3.41 (m, 1H), 3.62-3.73 (m, 1H), 3.87-3.93 (m, 2H), 4.49-4.58 (m, 1H), 4.84-4.94 (m, 1H), 7.15 (d, J=8.56 Hz, 2H), 7.22 (t, J=72.0 Hz, 1H), 7.43 (d, J=8.56 Hz, 1H).

### Example S51. Synthesis of Compound 52

Compound 52 was synthesized by General Procedure D using 2-trifluromethyl-1-bromoethane as the alkyl halide. MS (ESI) m/z [M+H]⁺: 420.16. ¹H NMR (400 MHz, CDCl₃) δ ppm 7.31-7.38 (m, 2H), 7.11-7.16 (m, 2H), 6.31-6.73 (m, 1H), 5.26 (q, J=7.21 Hz, 1H), 4.23-4.44 (m, 2H), 3.98-4.13 (m, 1H), 3.80-3.93 (m, 3H), 3.59 (t, J=11.07 Hz, 1H), 3.10 (dd, J=11.51, 3.75 Hz, 1H), 2.90-2.99 (m, 1H), 2.62-2.72 (m, 1H), 2.32 (dd, J=4.38, 2.38 Hz, 1H), 2.28 (dd, J=4.31, 2.31 Hz, 1H), 1.48 (d, J=7.25 Hz, 1H), 1.41 (d, J=7.13 Hz, 3H).

### Example S52. General Procedure E for the Synthesis of Final Compounds

To a solution of 6-methyl-8-(2-methylbutyl)hexahydro-4H-pyrazino[1,2-a]pyrimidine-4,7(6H)-dione (0.300 g, 1.184 mmol) in DMF (6 mL) stirred in a flask immersed in an ice/water bath was added cesium carbonate (0.771 g, 2.368 mmol, 2 eq,) followed by the appropriate alkyl halide (1.1 eq.). The flask was removed from the bath and stirred until TLC indicated complete consumption of starting material. The reaction mixture was poured in ice-cold water (70 mL) and aqueous layer was extracted with EtOAc (100 mL). The organic layer was washed with ice cold brine (50 mL×3), dried over anhydrous Na₂SO₄ and concentrated under reduced pressure. The crude product was purified by preparative HPLC to afford to give the final compound.

### Example S53. Synthesis of Compound 42

Compound 42 was synthesized by General Procedure E using 4-(bromomethyl)-2-chloro-1-(trifluoromethyl)benzene as the alkyl halide. MS (ESI) m/z [M+H]⁺: 362.2. ¹H NMR (400 MHz, DMSO-d6) δ 0.75-0.89 (m, 3H), 0.82-0.87 (m, 3H), 0.96-1.13 (m, 1H), 1.23-1.31 (m, 4H), 1.64-1.75 (m, 1H), 2.06-2.09 (m, 1H), 2.55-2.62 (m, 1H), 2.65-2.76 (m, 1H), 3.05-3.15 (m, 1H), 3.15-3.26 (m, 3H), 3.64-3.74 (m, 1H), 3.84-3.95 (m, 2H), 4.52-4.60 (m, 1H), 4.86-4.94 (m, 1H), 7.17 (t, J=8.76 Hz, 2H), 7.41 (dd, J=8.19, 5.82 Hz, 2H).

### Example S54. Synthesis of Compound 43

Compound 43 was synthesized by General Procedure E using 4-(bromomethyl)-2-chloro-1-(trifluoromethyl)benzene as the alkyl halide. MS (ESI) m/z [M+H]⁺: 446.2. ¹H NMR (400 MHz, DMSO-d6) 0.72-0.80 (m, 3H), 0.80-0.87 (m, 3H), 0.96-1.10 (m, 1H), 1.21-1.27 (m, 1H), 1.28-1.34 (m, 3H), 1.62-1.79 (m, 1H), 2.00-2.13 (m, 1H), 2.53-2.65 (m, 1H), 2.66-2.76 (m, 1H), 3.00-3.10 (m, 1H), 3.17-3.29 (m, 3H), 3.62-3.72 (m, 1H), 4.00-4.08 (m, 2H), 4.55-4.65 (m, 1H), 4.85-4.95 (m, 1H), 7.52-7.60 (m, 1H), 7.73 (s, 1H), 7.80-7.88 (m, 1H).

### Example S55. Synthesis of Compound 44

To a solution of 6-methyl-8-(2-methylbutyl)hexahydro-4H-pyrazino[1,2-a]pyrimidine-4,7(6H)-dione (0.420 g, 1.657 mmol) and 1H-indole-3-carbaldehyde (0.264 g, 1.823 mmol) in DCE (15 mL) was added acetic acid (1 mL, 1.657 mmol) and heated the reaction mixture at 80° C. for 1 h. To the resulting reaction mixture was added portion wise NaBH₄ (0.188 g, 4.973 mmol) and the reaction mixture was heated at 80° C. and stirred for 4 h. When TLC analysis (5% MeOH in DCM) indicated complete consumption of the starting material the reaction mixture was diluted with water (40 mL) and aqueous layer was extracted with DCM (100 mL). The organic layer was washed with brine (50 mL), dried over anhydrous Na₂SO₄ and concentrated under reduced pressure. The crude obtained was purified by column chromatography (Silica 100-200 mesh; 5% MeOH in DCM) followed by washing with water (30 mL) and dried under reduced pressure to afford compound 44 (0.250 g, 39% yield) as an off white solid. MS (ESI) m/z [M+H]⁺: 383.4. ¹H NMR (400 MHz, DMSO-d6) δ 0.70 (t, J=7.09 Hz, 3H), 0.75-0.82 (m, 3H), 0.91-1.11 (m, 1H), 1.22-1.31 (m, 3H), 1.57-1.72 (m, 1H), 1.97-2.07 (m, 1H), 2.55-2.70 (m, 2H), 2.83 (dt, J=10.91, 2.74 Hz, 1H), 2.95-3.07 (m, 1H), 3.10-3.26 (m, 3H), 3.54-3.69 (m, 1H), 3.96-4.04 (m, 1H), 4.06-4.15 (m, 1H), 4.54-4.64 (m, 1H), 4.84-4.95 (m, 1H), 6.94-7.02 (m, 1H), 7.04-7.13 (m, 1H), 7.29-7.40 (m, 2H), 7.65 (d, J=7.95 Hz, 1H), 10.95 (s, 1H).

### Example S55. Synthesis of Intermediate Compound 1-(4-fluorobenzyl)-6-methylhexahydro-4H-pyrazino[1,2-a]pyrimidine-4,7(6H) dione

To a solution of 6-methylhexahydro-4H-pyrazino[1,2-a]pyrimidine-4,7(6H)-dione (250 mg, 1.40 mmol) in DMF (3 mL) was added potassium carbonate (580 mg, 4.20 mmol) followed by 4-fluorobenzylbromide (0.320 g, 1.70 mmol) and stirred at 80° C. temperature for 3 h. After completion, the reaction mixture was monitored by TLC (5% MeOH in DCM). The reaction mixture was poured in ice-cold water (50 mL) and aqueous layer was extracted with EtOAc (50 mL). The organic layer was dried over anhydrous Na₂SO₄ and concentrated under reduced pressure. The crude obtained was purified by column chromatography (Silica 100-200 mesh; 5% MeOH in DCM) to afford 1-(4-fluorobenzyl)-6-methylhexahydro-4H-pyrazino[1,2-a]pyrimidine-4,7(6H) dione (160 mg, 70% yield) as a white solid. MS (ESI) m/z [M+H]⁺: 292.

### Example S56. Synthesis of Compound 45

To a solution of 1-(4-fluorobenzyl)-6-methylhexahydro-4H-pyrazino[1,2-a]pyrimidine-4,7(6H) dione (80 mg, 0.2739 mmol) in DMF (3 mL) under an ice cold bath at 0° C. was added NaH (20 mg, 0.2739 mmol) and stirred for 20 min then added (2-bromoethyl)cyclobutane (67 mg, 0.41 mmol) after 3 h. After complete consumption of starting material (monitored by TLC), the reaction mixture was quenched with ice cold water and extracted with ethyl acetate. The organic layer was dried over anhydrous Na₂SO₄ and concentrated under reduced pressure. The crude obtained was purified by column chromatography (Silica 100-200 mesh; 5% MeOH in DCM) to afford 8-(2-cyclobutylethyl)-1-(4-fluorobenzyl)-6-methylhexahydro-4H-pyrazino[1,2-a]pyrimidine-4,7(6H)-dione (13 mg, 16% yield) as a gummy liquid. MS (ESI) m/z [M+H]⁺: 374. ¹H NMR (400 MHz, CD₃Cl₃): δ 7.30-7.40 (m, 2H), 7.00-7.10 (m, 2H), 5.15-5.25 (m, 1H), 4.25-4.35 (m, 1H), 3.80-3.95 (m, 2H), 3.55-3.65 (m, 1H), 3.25-3.45 (m, 2H), 3.05-3.20 (m, 2H), 2.90-3.0 (m, 1H), 2.60-2.70 (m, 1H), 2.15-2.40 (m, 2H), 1.75-2.10 (m, 4H), 1.55-1.65 (m, 4H), 1.20-1.30 (m, 3H).

### Example S57. Synthesis of Compound 46

To a solution of 1-(4-fluorobenzyl)-6-methylhexahydro-4H-pyrazino[1,2-a]pyrimidine-4,7(6H) dione (80 mg, 0.2739 mmol) in DMF (3 mL) under an ice cold bath at 0° C. was added NaH (20 mg, 0.2739 mmol) and stirred for 20 min, then was added (2-bromoethyl)cyclopentane (72 mg, 0.41 mmol) after 3 hours, completion of starting material monitored by TLC, the reaction mixture was quenched with ice cold water and extracted with ethyl acetate. The organic layer was dried over anhydrous Na₂SO₄ and concentrated under reduced pressure. The crude obtained was purified by column chromatography (Silica 100-200 mesh; 5% MeOH in DCM) to afford 8-(2-cyclopentylethyl)-1-(4-fluorobenzyl)-6-methylhexahydro-4H-pyrazino[1,2-a]pyrimidine-4,7(6H)-dione as a gummy liquid. Example S58. Synthesis of Intermediate Compound 6-(fluoromethyl)-8-(2-methylbutyl)hexahydro-4H-pyrazino[1,2-a]pyrimidine-4,7(6H)-dione Hydrochloride salt.

Step 1: Synthesis of N-(2,2-diethoxyethyl)-2-methylbutan-1-amine. To stirred neat 2,2-diethoxyethan-1-amine (20.0 g, 0.137 mmol) was added 2-methylbutanal (11.60 g, 0.137 mmol) at room temperature and the reaction mixture was heated to 100° C. for 3 h. To the resulting reaction mixture was slowly added ethanol (200 mL) followed by NaBH₄ (15.40 g, 0.413 mmol) at room temperature and the reaction mixture was stirred for 16 h. After complete consumption of starting material (monitored by TLC). The reaction mixture was cooled to room temperature and slowly quenched with a saturated solution of NH₄C₁ (100 mL). The aq. layer was extracted with EtOAc (200 mL×2). The combined organic layer was washed with brine (400 mL), dried over Na₂SO₄, filtered and concentrated under reduced pressure to get crude compound. The crude obtained was purified by column chromatography (silica 100-200 mesh; 10% MeOH in DCM) to obtain N-(2,2-diethoxyethyl)-2-methylbutan-1-amine (25.8 g, 88% yield) colorless liquid. MS (ESI) m/z [M+H]⁺: 204.3. ¹H NMR (400 MHz, DMSO-d6) δ 0.80-0.89 (m, 6H) 1.11 (t, J=6.98 Hz, 6H) 1.35-1.48 (m, 2H) 2.28-2.32 (m, 1H) 2.41-2.45 (m, 1H) 2.55 (d, J=5.49 Hz, 2H) 3.42-3.52 (m, 2H) 3.57-3.65 (m, 2H) 4.49 (t, J=5.49 Hz, 1H).

Step 2: (9H-fluoren-9-yl)methyl (1-((2,2-diethoxyethyl)(2-methylbutyl)amino)-3-hydroxy-1-oxopropan-2-yl)carbamate. To a stirred solution of (((9H-fluoren-9-yl)methoxy)carbonyl)serine (15.0 g, 45.81 mmol) in dry DMF (150 mL) maintained at 0° C. was added HATU (26.0 g, 68.80 mmol), DIPEA (23.92 mL, 137.61 mmol) followed by N-(2,2-diethoxyethyl)-2-methylbutan-1-amine (12.10 g, 59.63 mmol). The reaction mixture was stirred at room temperature for 4 h. After complete consumption of starting material, the reaction mixture was quenched with ice cold water (500 mL) and the aqueous layer was extracted with EtOAc (250 mL×2). The combined organic layer was washed with cold H₂O (200 mL) followed by brine (200 mL), dried over Na₂SO₄ and concentrated under reduced pressure to provide the crude product. The crude material was purified by column chromatography (Silica 100-200 mesh; 80% EtOAc in hexanes) to afford (9H-fluoren-9-yl)methyl (1-((2,2-diethoxyethyl)(2-methylbutyl)amino)-3-hydroxy-1-oxopropan-2-yl)carbamate (21.0 g, 89.43% yield) as yellow sticky solid. MS (ESI) m/z [M+Na]+: 535.35.

Step 3: Synthesis of 2-amino-N-(2,2-diethoxyethyl)-3-hydroxy-N-(2-methylbutyl)propenamide. To a stirred solution of (9H-fluoren-9-yl)methyl (1-((2,2-diethoxyethyl)(2-methylbutyl)amino)-3-hydroxy-1-oxopropan-2-yl)carbamate (21.0 g, 41.01 mmol) in dry DCM (110 mL) maintained at 0° C. was added diethylamine (58 mL, 2.80 volume) and reaction mixture was stirred at room temperature for 3 h. After complete consumption of starting material (monitored by TLC), the reaction mixture was concentrated under reduced pressure to get crude product. The crude obtained was purified by column chromatography (Silica 100-200 mesh; 5% MeOH in DCM) to afford 2-amino-N-(2,2-diethoxyethyl)-3-hydroxy-N-(2-methylbutyl)propenamide (9.50 g, 80% yield) as yellow sticky solid. MS (ESI) m/z [M+H]⁺: 291.4.

Step 4: Synthesis of (9H-fluoren-9-yl)methyl (3-((1-((2,2-diethoxyethyl)(2-methylbutyl)amino)-3-hydroxy-1-oxopropan-2-yl)amino)-3-oxopropyl)carbamate. To a stirred solution of 3-((((9H-fluoren-9-yl)methoxy)carbonyl)amino)propanoic acid (9.50 g, 30.54 mmol) in dry DMF (95 mL) maintained at 0° C. was added HATU (17.40 g, 45.81 mmol), DIPEA (16.0 mL, 91.62 mmol) followed by 2-amino-N-(2,2-diethoxyethyl)-3-hydroxy-N-(2-methylbutyl)propanamide (13.20 g, 45.81 mmol) at room temperature and the reaction mixture was stirred for 16 h. After completion, the reaction mixture was quenched with ice cold water (200 mL) and the aqueous layer was extracted with EtOAc (200 mL×2). The organic layer was washed with cold H₂O (500 mL) followed by saturated brine (200 mL), dried over Na₂SO₄ and concentrated under reduced pressure. The crude compound was purified by column chromatography (Silica 100-200 mesh; 80% EtOAc in Hexanes) to afford (9H-fluoren-9-yl)methyl (3-((1-((2,2-diethoxyethyl)(2-methylbutyl)amino)-3-hydroxy-1-oxopropan-2-yl)amino)-3-oxopropyl)carbamate (8.0 g, 31.0% yield) as a viscous yellow oil. MS (ESI) m/z [M−H]: 582.2.

Step 5: Synthesis of (9H-fluoren-9-yl)methyl 6-(hydroxymethyl)-8-(2-methylbutyl)-4,7-dioxohexahydro-2H-pyrazino[1,2-a]pyrimidine-1(6H)-carboxylate. A stirred solution of (9H-fluoren-9-yl)methyl (3-((1-((2,2-diethoxyethyl)(2-methylbutyl)amino)-3-hydroxy-1-oxopropan-2-yl)amino)-3-oxopropyl)carbamate (8.0 g, 13.77 mmol) in formic acid (48.0 mL, 6.0 volume) at room temperature and reaction mixture was stirred for 16 h. After completion, the reaction mixture was concentrated under reduced pressure to afford (9H-fluoren-9-yl)methyl 6-(hydroxymethyl)-8-(2-methylbutyl)-4,7-dioxohexahydro-2H-pyrazino[1,2-a]pyrimidine-1(6H)-carboxylate (6.0 g, crude) as brown semi-solid. The crude compound was used as such for next reaction without further purification. MS (ESI) m/z [M+H]⁺: 492.2.

Step 6: Synthesis of 6-(hydroxymethyl)-8-(2-methylbutyl)hexahydro-4H-pyrazino[1,2-a]pyrimidine-4,7(6H)-dione. To a solution of (9H-fluoren-9-yl)methyl 6-(hydroxymethyl)-8-(2-methylbutyl)-4,7-dioxohexahydro-2H-pyrazino[1,2-a]pyrimidine-1(6H)-carboxylate (6.0 g, 12.20 mmol) in CH₂Cl₂ (36.0 mL) was added diethylamine (18.0 mL) at 0° C. and the reaction mixture was stirred at room temperature for 3 h. After complete consumption of the starting material (monitored by TLC), the reaction mixture was concentrated under reduced pressure to obtain the crude compound. The crude material was purified by column chromatography (Silica 100-200 mesh; 5% MeOH in DCM) to afford 6-(hydroxymethyl)-8-(2-methylbutyl)hexahydro-4H-pyrazino[1,2-a]pyrimidine-4,7(6H)-dione (3.0 g, 93.75% yield) as a viscous colorless oil. MS (ESI) m/z [M+H]⁺: 270.20.

Step 7: Synthesis of tert-butyl 6-(hydroxymethyl)-8-(2-methylbutyl)-4,7-dioxohexahydro-2H-pyrazino[1,2-a]pyrimidine-1(6H)-carboxylate. To a solution of 6-(hydroxymethyl)-8-(2-methylbutyl)hexahydro-4H-pyrazino[1,2-a]pyrimidine-4,7(6H)-dione (3.0 g, 11.15 mmol) in CH₂Cl₂ (60 mL) was added triethylamine (4.5 mL, 33.45 mmol) followed by Boc anhydride (3.78 mL, 16.72 mmol) at 0° C. and the reaction mixture was stirred at room temperature for 16 h. After complete consumption of the starting material (monitored by TLC), the reaction mixture was slowly quenched with ice cold water (30 mL) and extracted with DCM (40 mL). The organic layer was washed with brine (30 mL), dried over Na₂SO₄ and concentrated under reduced pressure. The crude compound was purified by column chromatography (Silica 100-200 mesh; 10% MeOH in DCM) to afford tert-butyl 6-(hydroxymethyl)-8-(2-methylbutyl)-4,7-dioxohexahydro-2H-pyrazino[1,2-a]pyrimidine-1(6H)-carboxylate (8.0 g, 31.0% yield) as a viscous yellow oil. MS (ESI) m/z [M+H]⁺: 370.25.

Step 8: Synthesis of tert-butyl 6-(fluoromethyl)-8-(2-methylbutyl)-4,7-dioxohexahydro-2H-pyrazino[1,2-a]pyrimidine-1(6H)-carboxylate. To a solution of 6-(hydroxymethyl)-8-(2-methylbutyl)-4,7-dioxohexahydro-2H-pyrazino[1,2-a]pyrimidine-1(6H)-carboxylate (1.50 g, 4.065 mmol) in DCM (30 mL) was added DAST (1.97 g, 12.19 mmol) at −78° C. and stirred for 15 min. The reaction mixture was allowed to warm to room temperature and stirred for 3 h. After completion of the reaction (monitored by TLC), the reaction mixture was quenched with saturated NaHCO₃ solution (15 mL) and the aqueous layer was extracted with EtOAc (100 mL×2). The combined organic layer was washed with saturated brine (50 mL), dried over Na₂SO₄ and concentrated under reduced pressure to obtain crude compound. The crude material was purified by column chromatography (Silica 100-200 mesh; 5% MeOH in DCM) to afford tert-butyl 6-(fluoromethyl)-8-(2-methylbutyl)-4,7-dioxohexahydro-2H-pyrazino[1,2-a]pyrimidine-1(6H)-carboxylate (0.800 g, 72.0% yield) as a colorless viscous oil. MS (ESI) m/z [M+H]⁺: 372.2.

Step 9: Synthesis of 6-(fluoromethyl)-8-(2-methylbutyl)hexahydro-4H-pyrazino[1,2-a]pyrimidine-4,7(6H)-dione Hydrochloride salt. To a stirred solution of tert-butyl 6-(fluoromethyl)-8-(2-methylbutyl)-4,7-dioxohexahydro-2H-pyrazino[1,2-a]pyrimidine-1(6H)-carboxylate (1.0 g, 2.695 mmol) in 1,4-dioxane (5 mL) was added 4 M HCl in dioxane (5 mL) at 0° C. and the reaction mixture was stirred at room temperature for 3 h. After complete consumption of the starting material (monitored by TLC), the reaction mixture was quenched with saturated solution of sodium bicarbonate (10 mL) and extracted with ethyl acetate (20 mL×3). The combined organic layer was washed with saturated brine (10 mL), dried over Na₂SO₄ and concentrated under reduced pressure to afford 6-(fluoromethyl)-8-(2-methylbutyl)hexahydro-4H-pyrazino[1,2-a]pyrimidine-4,7(6H)-dione hydrochloride salt (0.630 g, crude) as a brown sticky oil. MS (ESI) m/z [M+H]*free base: 271.00.

### Example S59. Synthesis of Compound 47

To a solution of 6-(fluoromethyl)-8-(2-methylbutyl)hexahydro-4H-pyrazino[1,2-a]pyrimidine-4,7(6H)-dione hydrochloride salt (0.150 g, 0.550 mmol) in DMF (1.5 mL) was added K₂CO₃ (0.381 g, 2.760 mmol) followed by 1-(bromomethyl)-4-(difluoromethoxy)benzene (0.261 g, 1.100 mmol) and the reaction mixture was stirred at room temperature for 16 h. After completion (monitored by TLC), the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to get crude compound. The crude compound was purified by Prep HPLC to afford 1-(4-(difluoromethoxy)benzyl)-6-(fluoromethyl)-8-(2-methylbutyl)hexahydro-4H-pyrazino[1,2-a]pyrimidine-4,7(6H)-dione (0.040 g, 17.0% yield) as a white solid. MS (ESI) m/z [M+H]⁺: 428.10. ¹H NMR (400 MHz, CDCl₃) δ 7.34 (d, J=8.01, 2 H), 7.11 (d, J=8.01, 2H), 6.32-6.69 (m, 1H), 5.14-5.25 (m, 2H), 4.60-4.76 (m, 2H), 3.84-3.97 (m, 2H), 3.35-3.45 (m, 2H), 3.12-3.40 (m, 4H), 2.85-3.05 (m, 1H), 2.65-2.75 (m, 1H), 2.29-2.34 (m, 1H), 1.65-1.75 (m, 1H), 1.30-1.40 (m, 1H), 1.05-1.18 (m, 1H), 0.80-0.90 (m, 6H).

### Example S60. Synthesis of Compound 48

To a solution of 6-(fluoromethyl)-8-(2-methylbutyl)hexahydro-4H-pyrazino[1,2-a]pyrimidine-4,7(6H)-dione hydrochloride salt (0.340 g, 1.253 mmol) in DMF (3.4 mL) was added Cs₂CO₃ (0.814 g, 2.506 mmol) followed by 1-(bromomethyl)-4-(trifluoromethyl)benzene (0.598 g, 2.506 mmol), and reaction mixture was stirred at room temperature for 16 h. After completion (monitored by TLC), the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to get crude compound. The crude compound was purified by prep HPLC to afford 6-(fluoromethyl)-8-(2-methylbutyl)-1-(4-(trifluoromethyl)benzyl)hexahydro-4H-pyrazino[1,2-a]pyrimidine-4,7(6H)-dione (0.045 g, 8.0% yield) as a white solid. MS (ESI) m/z [M+H]*: 430.10. ¹H NMR (400 MHz, CDCl₃) δ 7.34 (d, J=8.01, 2H), 7.11 (d, J=8.01, 2H), 5.14-5.25 (m, 2H), 4.60-4.76 (m, 2H), 3.84-3.97 (m, 2H), 3.35-3.45 (m, 2H), 3.12-3.40 (m, 4H), 2.85-3.05 (m, 1H), 2.65-2.75 (m, 1H), 2.29-2.34 (m, 1H), 1.65-1.75 (m, 1H), 1.30-1.40 (m, 1H), 1.05-1.18 (m, 1H), 0.80-0.90 (m, 6H).

### Example S61. Synthesis of Intermediate Compound methyl 2-(1-(4-(difluoromethoxy)benzyl)-8-(2-methylbutyl)-4,7-dioxooctahydro-2H-pyrazino[1,2-a]pyrimidin-6-yl)acetate

Step 1: Synthesis of methyl 3-((((9H-fluoren-9-yl)methoxy)carbonyl)amino)-4-((2,2-diethoxyethyl)(2-methylbutyl)amino)-4-oxobutanoate. To a solution 2-((((9H-fluoren-9-yl)methoxy)carbonyl)amino)-4-methoxy-4-oxobutanoic acid (1.90 g, 9.475 mmol) stirred at 0° C. in dry DMF (30 mL) was added HATU (3.60 g, 1.137 mmol) followed by DIPEA (2.70 mL, 1.895 mmol), and the reaction mixture was stirred at same temperature for 10 min. To the resulting reaction mixture was added N-(2,2-diethoxyethyl)-2-methylbutan-1-amine (3.50 g, 9.475 mmol), then the mixture was allowed to warm to room temperature and stirred for 6 h. After complete consumption of the starting material (monitored by TLC), the reaction mixture was quenched with ice cold water (100 mL) and the aqueous layer was extracted with EtOAc (50 mL×2). The combined organic layer was washed with cold H₂O (50 mL) followed by brine (50 mL), dried over Na₂SO₄ and concentrated under reduced pressure to get crude product. The crude material was purified by CombiFlash column chromatography using 50% EtOAc in n-hexanes to afford methyl 3-((((9H-fluoren-9-yl)methoxy)carbonyl)amino)-4-((2,2-diethoxyethyl)(2-methylbutyl)amino)-4-oxobutanoate (4.30 g, 83.0% yield) as a white solid. MS (ESI) m/z [M+H-EtOH]*: 509.2.

Step 2: Synthesis of methyl 3-amino-4-((2,2-diethoxyethyl)(2-methylbutyl)amino)-4-oxobutanoate. To a solution of methyl 3-((((9H-fluoren-9-yl)methoxy)carbonyl)amino)-4-((2,2-diethoxyethyl)(2-methylbutyl)amino)-4-oxobutanoate (1.36 g, 2.451 mmol) in CH₂Cl₂ (27.0 mL) was added diethylamine (1.53 mL, 14.71 mmol) at room temperature and the reaction mixture was stirred for 3 h. After complete consumption of the starting material (monitored by TLC), the reaction mixture was concentrated under reduced pressure to obtained crude compound. The crude compound was purified by CombiFlash column chromatography using 5% MeOH in DCM to afford methyl 3-amino-4-((2,2-diethoxyethyl)(2-methylbutyl)amino)-4-oxobutanoate (0.700 g, 86% yield) as yellow viscous liquid. MS (ESI) m/z [M+H-EtOH]+: 287.68.

Step 3: Synthesis of methyl 3-(3-((((9H-fluoren-9-yl)methoxy)carbonyl)amino)propanamido)-4-((2,2-diethoxyethyl)(2-methylbutyl)amino)-4-oxobutanoate. To a stirred solution of 3-((((9H-fluoren-9-yl)methoxy)carbonyl)amino)propanoic acid (0.490 g, 1.594 mmol) in dry DMF (10 mL) maintained at 0° C. was added HATU (0.720 g, 1.913 mmol), DIPEA (0.555 mL, 3.188 mmol) followed by the addition of methyl 3-amino-4-((2,2-diethoxyethyl)(2-methylbutyl)amino)-4-oxobutanoate (0.530 g, 1.594 mmol). The reaction mixture was allowed to warm to room temperature and stirred for 6 h. After completion, the reaction mixture was quenched with ice cold water (20 mL) and the aqueous layer was extracted with EtOAc (20 mL×2). The organic layer was washed with cold H₂O (10 mL) followed by saturated brine (20 mL), dried over Na₂SO₄ and concentrated under reduced pressure. The crude compound was purified by Combiflash column chromatography using 5% MeOH in DCM to afford methyl 3-(3-((((9H-fluoren-9-yl)methoxy)carbonyl)amino)propanamido)-4-((2,2-diethoxyethyl)(2-methylbutyl)amino)-4-oxobutanoate (0.630 g, 70% yield) as an off-white solid. MS (ESI) m/z [M+H-EtOH]+: 580.20.

Step 4: Synthesis of (9H-fluoren-9-yl)methyl 6-(2-methoxy-2-oxoethyl)-8-(2-methylbutyl)-4,7-dioxohexahydro-2H-pyrazino[1,2-a]pyrimidine-1(6H)-carboxylate. To a stirred solution of methyl 3-(3-((((9H-fluoren-9-yl)methoxy)carbonyl)amino)propanamido)-4-((2,2-diethoxyethyl)(2-methylbutyl)amino)-4-oxobutanoate (0.300 g, 0.4794 mmol) was added formic acid (1.5 mL) at room temperature and the reaction mixture was stirred for 16 h. After completion, the reaction mixture was concentrated and the crude obtained was purified by column chromatography (Silica 100-200 mesh; 0-5% MeOH in DCM) to afford (9H-fluoren-9-yl)methyl 6-(2-methoxy-2-oxoethyl)-8-(2-methylbutyl)-4,7-dioxohexahydro-2H-pyrazino[1,2-a]pyrimidine-1(6H)-carboxylate (0.200 g, 80% yield) as a yellow solid. MS (ESI) m/z [M+H]*: 534.67.

Step 5: Synthesis of methyl 2-(8-(2-methylbutyl)-4,7-dioxooctahydro-2H-pyrazino[1,2-a]pyrimidin-6-yl)acetate. To a solution of (9H-fluoren-9-yl)methyl 6-(2-methoxy-2-oxoethyl)-8-(2-methylbutyl)-4,7-dioxohexahydro-2H-pyrazino[1,2-a]pyrimidine-1(6H)-carboxylate (0.240 g, 0.4499 mmol) in CH₂Cl₂ (0.5 mL) was added diethylamine (0.280 mL) and the reaction mixture was stirred at room temperature for 3 h. After complete consumption of the starting material (monitored by TLC), the reaction mixture was concentrated and the crude material was purified by combiflash column chromatography using 0-5% MeOH in DCM to afford methyl 2-(8-(2-methylbutyl)-4,7-dioxooctahydro-2H-pyrazino[1,2-a]pyrimidin-6-yl)acetate (0.130 g, 93% yield) as white solid. MS (ESI) m/z [M−H]+: 310.4.

Step 6: Synthesis of methyl 2-(1-(4-(difluoromethoxy)benzyl)-8-(2-methylbutyl)-4,7-dioxooctahydro-2H-pyrazino[1,2-a]pyrimidin-6-yl)acetate. To a solution of methyl 2-(8-(2-methylbutyl)-4,7-dioxooctahydro-2H-pyrazino[1,2-a]pyrimidin-6-yl)acetate (3.08 g, 9.890 mmol) in DMF (30 mL) was added K₂CO₃ (4.10 g, 29.66 mmol) at room temperature, and reaction mixture stirred at 80° C. for 15 min. To the resulting reaction mixture was added 1-(bromomethyl)-4-(difluoromethoxy)benzene (3.48 g, 14.36 mmol) and the stirred mixture was heated to 80° C. for 2 h. After completion, the reaction mixture was quenched with ice cold water (200 mL) and the aqueous layer was extracted with EtOAc (200 mL×2). The organic layer was washed with cold H₂O (200 mL) followed by saturated brine (150 mL), dried over Na₂SO₄ and concentrated under reduced pressure. The crude compound obtained was purified by Combiflash column chromatography (5% MeOH in DCM) to afford methyl 2-(1-(4-(difluoromethoxy)benzyl)-8-(2-methylbutyl)-4,7-dioxooctahydro-2H-pyrazino[1,2-a]pyrimidin-6-yl)acetate (2.20 g, 48% yield) as a yellow solid. MS (ESI) m/z [M-CH₃]⁺: 454.10.

### Example S61. Synthesis of Compound 49

To a solution of methyl 2-(1-(4-(difluoromethoxy)benzyl)-8-(2-methylbutyl)-4,7-dioxooctahydro-2H-pyrazino[1,2-a]pyrimidin-6-yl)acetate (2.20 g, 4.705 mmol) in THE (22.0 mL) was added NaOH (0.560 g, 14.11 mmol) followed by water (4 mL) and the reaction mixture was stirred at room temperature for 3 h. Progress of the reaction was monitored by TLC. After completion, the reaction mixture was concentrated under reduced pressure. The crude residue was dissolved in water (10 mL), slowly acidified with 6N HCl (10 mL) and stirred for 5 min. The obtained solid precipitate was filtered through a Buchner funnel and dried under reduced pressure to afford 2-(1-(4-(difluoromethoxy)benzyl)-8-(2-methylbutyl)-4,7-dioxooctahydro-2H-pyrazino[1,2-a]pyrimidin-6-yl)acetic acid (0.85 g, 40% yield) as a white solid. MS (ESI) m/z [M+H]⁺: 454.10. ¹H NMR (400 MHz, CDCl₃) δ 7.28-7.38 (m, 2H), 7.11 (d, J=7.99 Hz, 2H), 6.33-6.71 (m, 1H), 5.36-5.40 (m, 1H), 4.70-4.80 (m, 1H), 4.65-4.75 (m, 1H), 3.80-4.00 (m, 2H), 3.55-3.65 (m, 1H), 3.35-3.45 (m, 1H), 2.85-3.30 (m, 6H), 2.70-2.80 (m, 1H), 2.25-2.35 (m, 1H), 1.65-1.76 (m, 1H), 1.25-1.35 (m, 1H), 1.10-1.20 (m, 1H), 0.8-0.9 (m, 6H).

### Example S62. Synthesis of Compound 50

To a solution of 2-(1-(4-(difluoromethoxy)benzyl)-8-(2-methylbutyl)-4,7-dioxooctahydro-2H-pyrazino[1,2-a]pyrimidin-6-yl)acetic acid (0.470 g, 1.036 mmol) in THE (5 mL) was added 1,1′-carbonyldiimidazole (0.500 g, 3.109 mmol) at room temperature and the reaction mixture was stirred for 15 min. To the resulting reaction mixture was added aq. NH₃ (10 mL) and reaction mixture was stirred at room temperature for 3 h. Progress of the reaction was monitored by TLC. After completion, the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to provide the crude compound. The crude compound obtained was purified by Combiflash column chromatography using 5% MeOH in DCM followed by PREP HPLC to afford 2-(1-(4-(difluoromethoxy)benzyl)-8-(2-methylbutyl)-4,7-dioxooctahydro-2H-pyrazino[1,2-a]pyrimidin-6-yl)acetamide (0.070 g, 15% yield) as a white solid. MS (ESI) m/z [M+H]⁺: 453.20. ¹H NMR (400 MHz, CDCl₃) δ 7.30-7.40 (m, 2H), 7.05-7.15 (m, 2H), 6.39-6.70 (m, 1H), 5.20-5.40 (m, 2H), 4.75-4.85 (m, 1H), 3.95-4.05 (m, 1H), 3.75-3.85 (m, 1H), 3.50-3.60 (m, 1H), 3.30-3.40 (m, 1H), 3.05-3.25 (m, 2H), 2.85-2.95 (m, 2H), 2.55-2.70 (m, 1H), 2.25-2.35 (m, 1H), 1.70-1.80 (m, 2H), 1.30-1.40 (m, 2H), 1.05-1.20 (m, 2H), 0.75-0.90 (m, 6H).

### Example S63. Synthesis of Intermediate Compound 1-(3-chloro-4-(trifluoromethyl)benzyl)-6-methylhexahydro-4H-pyrazino[1,2-a]pyrimidine-4,7(6H)-dione

To a solution of 6-methylhexahydro-4H-pyrazino[1,2-a]pyrimidine-4,7(6H)-dione (500 mg, 2.732 mmol) in DMF (7 mL) was added potassium carbonate (1.13 g, 8.196 mmol) followed by 4-(bromomethyl)-2-chloro-1-(trifluoromethyl)benzene (0.894 g, 3.278 mmol) and stirred at 80° C. temperature for 12 h. After completion of the reaction, monitored by TLC (5% MeOH in DCM). The reaction mixture was poured in ice-cold water (50 mL) and the aqueous layer was extracted with EtOAc (50 mL). The organic layer was dried over anhydrous Na₂SO₄ and concentrated under reduced pressure. The crude obtained was purified by column chromatography (Silica 100-200 mesh; 5% MeOH in DCM) to afford 1-(3-chloro-4-(trifluoromethyl)benzyl)-6-methylhexahydro-4H-pyrazino[1,2-a]pyrimidine-4,7(6H)-dione (320 mg, 42% yield) as a white solid. MS (ESI) m/z [M+H]⁺: 376.34.

### Example S64. General Procedure F for the Synthesis of Final Compounds

To a solution of 1-(3-chloro-4-(trifluoromethyl)benzyl)-6-methylhexahydro-4H-pyrazino[1,2-a]pyrimidine-4,7(6H)-dione (150 mg, 0.400 mmol) in DMF (2 mL) at 0° C. was added Cs₂CO₃ (4 eq) and stirred for 20 min, then was added the appropriate alkyl halide (1.2 eq) at room temperature and the reaction mixture was heated at 80° C. and stirred for 12 h. After consumption of starting material (monitored by TLC), the reaction mixture was quenched with ice cold water and extracted with ethyl acetate. The organic layer was dried over anhydrous Na₂SO₄ and concentrated under reduced pressure. The crude obtained was purified by column chromatography (Silica 100-200 mesh; 5% MeOH in DCM) to give the final compounds.

### Example S65. Synthesis of Compound 51

Compound 51 was synthesized by General Procedure F using (2-bromoethyl)cyclobutane as the alkyl halide. MS (ESI) m/z [M+H]⁺: 458.2. ¹H NMR (400 MHz, CDCl₃) δ ppm 7.70 (d, J=8.07 Hz, 1H), 7.54 (s, 1H), 7.33 (d, J=8.68 Hz, 1H), 4.34 (dd, J=10.64, 3.55 Hz, 1H), 3.87-3.99 (m, 2H), 3.61 (t, J=11.13 Hz, 1H), 3.25-3.42 (m, 2H), 3.08-3.19 (m, 2H), 2.89-2.98 (m, 1H), 2.64-2.74 (m, 1H) 2.29-2.38 (m, 1H) 2.17-2.27 (m, 1H) 1.97-2.09 (m, 2H) 1.72-1.92 (m, 3H) 1.58-1.66 (m, 4H) 1.55 (br. s, 3H).

### Example S66. Synthesis of Compound 54

Compound 54 was synthesized by General Procedure F using (2-bromoethyl)cyclopentane as the alkyl halide. MS (ESI) m/z [M+H]⁺: 472.15. ¹H NMR (400 MHz, CDCl₃) δ ppm 7.69 (d, J=8.11 Hz, 1H) 7.52-7.56 (m, 1H) 7.33 (d, J=7.89 Hz, 1H), 5.23 (q, J=7.23 Hz, 1H), 4.36 (dd, J=10.52, 3.29 Hz, 1H), 3.87-3.98 (m, 2H), 3.63 (t, J=11.07 Hz, 1H), 3.46-3.56 (m, 1H), 3.25-3.35 (m, 1H), 3.12-3.23 (m, 2H), 2.87-2.97 (m, 1H), 2.60-2.70 (m, 1H), 2.29-2.37 (m, 1H), 1.66-1.82 (m, 2H), 1.54-1.63 (m, 1H), 1.51 (d, J=2.63 Hz, 2H), 1.42 (d, J=7.23 Hz, 3H), 1.26 (br. s, 2H) 1.04-1.16 (m, 2H) 0.80-0.92 (m, 2H).

### Example S67. Synthesis of Compound 53

Step 1: Synthesis of (9H-fluoren-9-yl)methyl (1-((2,2-diethoxyethyl)(2-methylbutyl)amino)-4-methyl-1-oxopentan-2-yl)carbamate. To a stirred solution of (((9H-fluoren-9-yl)methoxy)carbonyl)leucine (20.0 g, 56.58 mmol) in dry DMF (200 mL) was added HATU (21.50 g, 56.58 mmol) followed by DIPEA (10.62 mL, 61.10 mmol) at 0° C. and the reaction mixture was stirred at same temperature for 10 min. To the resulting reaction mixture was added N-(2,2-diethoxyethyl)-2-methylbutan-1-amine (11.48 g, 56.58 mmol) at room temperature and the reaction mixture was stirred for 3 h. After complete consumption of the starting material (monitored by TLC), the reaction mixture was quenched with ice cold water (100 mL) and the aqueous layer was extracted with EtOAc (50 mL×4). The combined organic layers were washed with cold H₂O (50 mL×2) followed by brine (50 mL), dried over Na₂SO₄ and concentrated under reduced pressure to get crude product. The crude product was purified by CombiFlash column chromatography using 5% MeOH in DCM to afford (9H-fluoren-9-yl)methyl (1-((2,2-diethoxyethyl)(2-methylbutyl)amino)-4-methyl-1-oxopentan-2-yl)carbamate (14.5 g, 47.57% yield) as a white solid. MS (ESI) m/z [M+H]⁺: 539.04.

Step 2: Synthesis of 2-amino-N-(2,2-diethoxyethyl)-4-methyl-N-(2-methylbutyl)pentanamide. To a solution of (9H-fluoren-9-yl)methyl (1-((2,2-diethoxyethyl)(2-methylbutyl)amino)-4-methyl-1-oxopentan-2-yl)carbamate (8.50 g, 15.77 mmol) in CH₂Cl₂ (50 mL) was added diethylamine (16 mL, 157.7 mmol) at room temperature and the reaction mixture was stirred for 3 h. After complete consumption of the starting material (monitored by TLC), the reaction mixture was concentrated under reduced pressure to obtained crude compound. The crude compound was purified by CombiFlash column chromatography using 5% MeOH in DCM to afford 2-amino-N-(2,2-diethoxyethyl)-4-methyl-N-(2-methylbutyl)pentanamide (3.60 g, 72% yield) as a yellow viscous liquid. MS (ESI) m/z [M+H-EtOH]+: 272.10.

Step 3: Synthesis of (9H-fluoren-9-yl)methyl (3-((1-((2,2-diethoxyethyl)(2-methylbutyl)amino)-4-methyl-1-oxopentan-2-yl)amino)-3-oxopropyl)carbamate. To a stirred solution of 3-((((9H-fluoren-9-yl)methoxy)carbonyl)amino)propanoic acid (3.80 g, 12.28 mmol) in dry DMF (35 mL) maintained at 0° C. was added HATU (6.48 g, 17.05 mmol) and DIPEA (4.90 mL, 28.42 mmol), followed by the addition of 2-amino-N-(2,2-diethoxyethyl)-4-methyl-N-(2-methylbutyl)pentanamide (3.60 g, 11.37 mmol). The reaction mixture was allowed to attain room temperature and stirred for 3 h. After completion, the reaction mixture was quenched with ice cold water (20 mL) and the aqueous layer was extracted with EtOAc (30 mL×2). The organic layer was washed with cold H₂O (10 mL) followed by saturated brine (20 mL), dried over Na₂SO₄ and concentrated under reduced pressure. The crude compound was purified by Combiflash column chromatography using 5% MeOH in DCM to afford (9H-fluoren-9-yl)methyl (3-((1-((2,2-diethoxyethyl)(2-methylbutyl)amino)-4-methyl-1-oxopentan-2-yl)amino)-3-oxopropyl)carbamate (3.8 g, 55% yield) as an off-white solid. MS (ESI) m/z [M+H-EtOH]*: 565.30.

Step 4: Synthesis of (9H-fluoren-9-yl)methyl 6-isobutyl-8-(2-methylbutyl)-4,7-dioxohexahydro-2H-pyrazino[1,2-a]pyrimidine-1(6H)-carboxylate. To a stirred solution of (9H-fluoren-9-yl)methyl 6-isobutyl-8-(2-methylbutyl)-4,7-dioxohexahydro-2H-pyrazino[1,2-a]pyrimidine-1(6H)-carboxylate (3.80 g, 6.231 mmol) was added formic acid (20 mL) at room temperature and the reaction mixture was stirred for 16 h. After completion, the reaction mixture was concentrated under reduced pressure. The crude compound was purified by column chromatography (Silica 100-200 mesh; 0-5% MeOH in DCM) to afford (9H-fluoren-9-yl)methyl 6-isobutyl-8-(2-methylbutyl)-4,7-dioxohexahydro-2H-pyrazino[1,2-a]pyrimidine-1(6H)-carboxylate (3.60 g, 94% yield) as a yellow solid. MS (ESI) m/z [M+H]⁺: 518.23.

Step 5: Synthesis of 6-isobutyl-8-(2-methylbutyl)hexahydro-4H-pyrazino[1,2-a]pyrimidine-4,7(6H)-dione. To a solution of (9H-fluoren-9-yl)methyl 6-isobutyl-8-(2-methylbutyl)-4,7-dioxohexahydro-2H-pyrazino[1,2-a]pyrimidine-1(6H)-carboxylate (3.60 g, 6.954 mmol) in CH₂Cl₂ (36 mL) was added diethylamine (6.8 mL, 69.54 mmol) and the reaction mixture was stirred at room temperature for 16 h. After complete consumption of the starting material (monitored by TLC), the reaction mixture was concentrated under reduced pressure and the crude product was purified by combiflash column chromatography using 10-50% ethyl acetate in n-hexane to afford 6-isobutyl-8-(2-methylbutyl)hexahydro-4H-pyrazino[1,2-a]pyrimidine-4,7(6H)-dione (1.20 g, 60% yield) as a white solid. MS (ESI) m/z [M+H]⁺: 296.10.

Step 6: Synthesis of 1-(4-(difluoromethoxy)benzyl)-6-isobutyl-8-(2-methylbutyl)hexahydro-4H-pyrazino[1,2-a]pyrimidine-4,7(6H)-dione. To a solution of 6-isobutyl-8-(2-methylbutyl)hexahydro-4H-pyrazino[1,2-a]pyrimidine-4,7(6H)-dione (0.170 g, 0.576 mmol) in DMF (5 mL) was added K₂CO₃ (0.159 g, 1.152 mmol) at 0° C., and the reaction mixture was stirred for 10 min. To the resulting reaction mixture was added 1-(bromomethyl)-4-(difluoromethoxy)benzene (0.150 g, 0.632 mmol) at room temperature and stirred for 3 h. After completion, the reaction mixture was quenched with ice cold water (200 mL) and the aqueous layer was extracted with EtOAc (20 mL×2). The organic layer was washed with cold H₂O (20 mL) followed by saturated brine (15 mL), dried over Na₂SO₄ and concentrated under reduced pressure. The resulting crude compound was purified by PREP HPLC to afford 1-(4-(difluoromethoxy)benzyl)-6-isobutyl-8-(2-methylbutyl)hexahydro-4H-pyrazino[1,2-a]pyrimidine-4,7(6H)-dione (0.103 g, 40% yield) as a white solid. MS (ESI) m/z [M+H]: 452.3. ¹H NMR (400 MHz, DMSO d6) δ 7.42 (d, J=8.8 Hz, 2H), 7.14-7.24 (m, 3H), 5.0-5.10 (m, 1H), 4.50-4.60 (m, 1H), 3.90-4.00 (m, 2H), 3.60-3.70 (m, 1H), 3.02-3.40 (m, 4H), 2.70-2.85 (m, 2H), 2.0-2.10 (m, 1H), 1.50-1.70 (m, 4H), 1.20-1.35 (m, 1H), 1.0-1.10 (m, 1H), 0.70-0.98 (m, 12H).

### Biological Examples

### Example B1. Phospho-MET ELISA

Compounds were screened for potency towards the HGF/MET system using phospho-MET (pMET) ELISA kits (Cell Signaling). pMET levels were detected in samples having low (1 ng/mL) and high (10 ng/mL) concentrations of HGF.

HEK293 cells were prepared by passage into 6-well multi-plates and grown at 37° C. at 5% CO₂ in DMEM+10% FBS until approximately 90% confluent. Cells were then starved for at least 8 hours in serum-free growth media.

Exemplary compounds were prepared in DMEM+0.1% FBS, diluted and added to treatment media with 1 ng/mL recombinant HGF protein (R&D Systems). Cells were incubated in triplicate at 37° C. and 5% CO₂ for 15 minutes. Samples were then treated with 180 μL ice-cold RIPA (radioimmunoprecipitation assay) buffer and cells were lysed on ice for 15 minutes. Lysates were cleared by centrifugation at 16,000-g for 15 minutes and the supernatant was retained. Samples were normalized using a BCA assay of lysates to determine protein concentrations across the samples.

Between 50 and 100 μg total protein lysate was loaded into ELISA wells in pMET Sandwich ELISA kit (Cell Signaling Catalog #7227C), ensuring equal protein load in each well. The ELISA was processed according to manufacturer's instructions. After color developed, absorbance was read on an optical plate reader at 450 nm.

Potency measurements were determined using peak efficacy by scaling test compound dose treatments along a scale of 1-10 between 1 ng/mL and 10 ng/mL HGF doses according to the following formula:

$$y = 1 + ( x - A ) * ( 10 - 1 ) / ( B - A )$$

where y is the normalized data-point, x is the raw data point, A is the mean HGF at 1 ng/mL, and B is the mean HGF at 10 ng/mL. The results for the calculated potency are shown in Table 2.

### Table 2. Potency of Exemplary Compounds.

TABLE 2
Potency of Exemplary Compounds.

| Compound | Potency | Compound No. | Potency |
|---|---|---|---|
| 1a | ++++ | 2a | ++ |
| 3a | − | 4a | + |
| 5a | + | 6a | + |
| 7a | ++ | 8a | + |
| 9 | − | 10 | − |
| 11 | +++ | 12 | ++ |
| 13 | − | 14 | ++++ |
| 15 | − | 16 | − |
| 17 | +++ | 18 | − |
| 19 | +++ | 20 | + |
| 21 | − | 22 | +++ |
| 23 | − | 24 | − |
| 25 | − | 26 | − |
| 27 | − | 28 | − |
| 29 | − | 30 | +++ |
| 31 | − | 32 | − |
| 33 | − | 34 | − |
| 35 | − | 36 | − |
| 37 | − | 38 | − |
| 39 | − | 40 | − |
| 41 | − | 42 | ++++ |
| 43 | − | 44 | − |
| 45 | − | 46 | − |
| 47 | − | 48 | − |
| 49 | ++++ | 50 | − |
| 51 | +++ | 52 | − |
| 53 | ++ | 54 | − |

− indicates that the compound failed to significantly augment MET phosphorylation
+ indicates maximum potency at or above 100 nM
++ indicates maximum potency at or above 10 nM
+++ indicates maximum potency at or above 1 nM
++++ indicates maximum potency at or above 0.1 nM

− indicates that the compound failed to significantly augment MET phosphorylation
+ indicates maximum potency at or above 100 nM
++ indicates maximum potency at or above 10 nM
+++ indicates maximum potency at or above 1 nM
++++ indicates maximum potency at or above 0.1 nM

### Example B2. Cell Scattering Behavior Assay

MDCK cells were grown under normal conditions and observed to spontaneously form tight colonies as they proliferate. MDCK cells respond to HGF treatment by moving away from each other (scattering), which is quantified to assess the amount of HGF/MET activation in the cell population. In this experiment, MDCK cells were plated in a 96-well format, treated with HGF and exemplary compounds, fluorescently stained, imaged in large fields, and scattering behavior was quantified. Quantification was determined by analyzing the number of continuous groups of cells compared to the total stained area imaged (normalized particle counts).

MDCK cells were plated at low density in black-walled imaging plates and allowed to attach overnight at 37° C. and 5% CO₂ in DMEM+10% FBS. Cells were then starved to 2 hours in DMEM without FBS (“starve media”). Samples containing exemplary compounds were prepared in DMEM without FBS and included 5 ng/mL HGF protein (“treatment media”). A control curve was also prepared for each plate using HGF concentrations of 0, 5, 10, and 20 ng/mL. Starve media was replaced with treatment media and cells were incubated for 24 hours at 37° C. and 5% CO₂.

After incubation, cells were fixed by replacing treatment media with cold ethanol and incubating for 20 minutes at 4° C. Cells were then rehydrated by washing with PBS and then with stain solution (fluorescent wheat germ agglutinin; WGA488 at 20 μg/mL in PBS). Cells were incubated with stain solution 30 minutes at room temperature after which stain solution was replaced with fresh PBS.

Fields of cells were imaged using an iCyte high content imager in the green wavelength. Images were converted to binary and analyzed for particle size and particle count. For the purpose of analysis, an individual cell touching no other cells or separated colonies of cells were identified as particles, and particle counts were normalized by the total signal area to account for differences in cell number. An increase in the number of particles indicated that individual cells moved away from each other in a scattering behavior response. Compound potency was assessed by statistical increase in normalized particle count compared to HGF treatment alone. The results are shown in Table 3.

### Table 3. Cell Scattering Assay Results of Exemplary Compounds.

TABLE 3
Cell Scattering Assay Results of Exemplary Compounds.

| Compound | Potency | Compound | Potency |
|---|---|---|---|
| 1a | ++++ | 2a | + |
| 3a | − | 4a | + |
| 5a | ++++ | 6a | +++ |
| 7a | ++ | 8a | +++ |
| 9 | − | 10 | − |
| 11 | ++ | 12 | ++ |
| 13 | − | 14 | +++ |
| 15 | − | 16 | − |
| 17 | − | 18 | − |
| 19 | +++ | 20 | +++ |
| 21 | − | 22 | ++ |
| 23 | − | 24 | − |
| 25 | − | 26 | − |
| 27 | NT | 28 | − |
| 29 | NT | 30 | ++ |
| 31 | − | 32 | − |
| 33 | − | 34 | − |
| 35 | − | 36 | − |
| 37 | − | 38 | NT |
| 39 | − | 40 | NT |
| 41 | − | 42 | +++ |
| 43 | − | 44 | − |
| 45 | NT | 46 | − |
| 47 | NT | 48 | NT |
| 49 | ++++ | 50 | − |
| 51 | +++ | 52 | − |
| 53 | +++ | 54 | − |

− indicates that the compound failed to significantly promote cell scattering behavior
+ indicates maximum potency at or above 100 nM
++ indicates maximum potency at or above 10 nM
+++ indicates maximum potency at or above 1 nM
++++ indicates maximum potency at or above 0.1 nM
NT indicates the compound was not tested

− indicates that the compound failed to significantly promote cell scattering behavior
+ indicates maximum potency at or above 100 nM
++ indicates maximum potency at or above 10 nM
+++ indicates maximum potency at or above 1 nM
++++ indicates maximum potency at or above 0.1 nM
NT indicates the compound was not tested

### Example B3. Solubility Assay

Aqueous solubility is a critical drug property that helps to predict bioavailability. Generally, compounds with aqueous solubility <100 μg/ml are poor drugs. To assess compound solubility, a turbidimetric solubility assay was performed with exemplary compounds at a concentration range from 3-300 μM.

To assess a compound's solubility by turbidity, test compounds were first dissolved in organic solvent (DMSO) at a concentration of 10 mM. This compound solution was then diluted in aqueous solvent (PBS) in a dilution series from 3 to 300 μM in a 96-well assay plate. Solutions were incubated at 37° C. for 2 hours.

In wells with test compounds over their solubility limit, the compound will precipitate, effectively blocking the passage of light and thus increasing the absorbance signal of UV light at a wavelength of 620 nm. Compounds were considered insoluble at a tested concentration if turbidity raises the absorbance more than 10% above control reads. The results are shown in Table 4.

### Table 4. Solubility of Exemplary Compounds.

TABLE 4
Solubility of Exemplary Compounds.

| Compound | Solubility | Compound | Solubility |
|---|---|---|---|
| 1a | ++++ | 2a | ++++ |
| 3a | ++++ | 4a | ++++ |
| 5a | ++++ | 6a | ++++ |
| 7a | ++++ | 8a | ++++ |
| 9 | ++++ | 10 | ++++ |
| 11 | ++++ | 12 | ++++ |
| 13 | ++++ | 14 | ++++ |
| 15 | ++++ | 16 | ++++ |
| 17 | ++++ | 18 | ++++ |
| 19 | ++++ | 20 | ++++ |
| 21 | ++++ | 22 | ++++ |
| 23 | ++++ | 24 | ++++ |
| 25 | ++++ | 26 | ++++ |
| 27 | +++ | 28 | +++ |
| 29 | ++++ | 30 | ++++ |
| 31 | ++++ | 32 | ++++ |
| 33 | ++++ | 34 | ++++ |
| 35 | ++++ | 36 | ++++ |
| 37 | ++++ | 38 | +++ |
| 39 | ++++ | 40 | ++++ |
| 41 | ++++ | 42 | ++++ |
| 43 | +++ | 44 | +++ |
| 45 | ++++ | 46 | ++++ |
| 47 | ++++ | 48 | +++ |
| 49 | ++++ | 50 | ++++ |
| 51 | +++ | 52 | +++ |
| 53 | +++ | 54 | ++ |

+ indicates solubility at 10 μM
++ indicates solubility at 30 μM
+++ indicates solubility at 100 μM
++++ indicates solubility at 300 μM

+ indicates solubility at 10 μM
++ indicates solubility at 30 μM
+++ indicates solubility at 100 μM
++++ indicates solubility at 300 μM

### Example B4. Permeability Assay

Bioavailable drugs must permeate the cellular membranes of the lining of the digestive tract. To estimate the penetrability of exemplary compounds, the in vitro parallel artificial membrane permeability assay (PAMPA) was utilized.

Test compounds must have a standard curve in the final read plate to determine partitioned concentration of each drug. A 6-point standard curve was prepared for each compound from 0 to 200 μM in phosphate-buffered saline (PBS).

Test compound solution (300 μL in PBS) was added to the donor (bottom) well of the PAMPA plate in 5 replicates and PBS vehicle (200 μL) was added to the acceptor (top) wells of appropriate wells to match the loading of the donor plate. The bottom and top of the PAMPA plates were then sandwiched together. The PAMPA plates were then incubated at room temperature for 5 hours. After incubation, 150 μL of donor solution was added to a UV compatible plate containing the corresponding standard curve. 150 μL of acceptor well solution was added adjacent to the corresponding standard curve and donor well samples for that compound. The plate was then read using a UV plate reader.

Permeability and membrane retention were then calculated based on the following formulas:

$$Permeability ( cm / s ) : ( Pe ) ( cm / s ) = \{ - ln [ 1 - C A ( t ) / Ceq ] \} / [ A * ( 1 / VD + 1 / VA ) * t ] \qquad ( equation 1 )$$

where:
A=filter area (0.3 cm²);
VD=donor well volume (0.3 mL);
VA=acceptor well volume (0.2 mL);
t=incubation time (seconds);
CA(t)=compound concentration in acceptor well at time t;
CD(t)=compound concentration in donor well at time t; and

$$Ceq = [ CD ( t ) * VD + CA ( t ) * VA ] / ( VD + V A ) . \qquad ( equation 2 )$$

$$Membrane Retention ( R ) = 1 - [ CD ( t ) * VD + CA ( t ) * VA ] / ( C 0 * VD )$$

where:
CD(t), VD, CA(t), and VA are as defined for equation 1, and
C₀=initial concentration in donor well (200 uM).

The results are shown in Table 5.

### Table 5. Permeability of Exemplary Compounds.

TABLE 5
Permeability of Exemplary Compounds.

| Compound | Permeability | Compound | Permeability |
|---|---|---|---|
| 1a | ++ | 2a | +++ |
| 3a | +++ | 4a | + |
| 5a | +++ | 6a | +++ |
| 7a | + | 8a | + |
| 9 | ++ | 10 | NT |
| 11 | ++ | 12 | +++ |
| 13 | NT | 14 | ++ |
| 15 | NT | 16 | NT |
| 17 | ++ | 18 | NT |
| 19 | +++ | 20 | +++ |
| 21 | NT | 22 | +++ |
| 23 | NT | 24 | NT |
| 25 | NT | 26 | NT |
| 27 | NT | 28 | NT |
| 29 | NT | 30 | +++ |
| 31 | NT | 32 | NT |
| 33 | NT | 34 | NT |
| 35 | NT | 36 | NT |
| 37 | NT | 38 | NT |
| 39 | NT | 40 | NT |
| 41 | NT | 42 | ++ |
| 43 | NT | 44 | NT |
| 45 | NT | 46 | NT |
| 47 | NT | 48 | NT |
| 49 | ++ | 50 | NT |
| 51 | +++ | 52 | NT |
| 53 | +++ | 54 | +++ |

+ indicates permeability above 1 × 10⁻⁵ cm/s
++ indicates permeability above 2 × 10⁻⁶ cm/s
+++ indicates permeability below 2 × 10⁻⁶ cm/s
NT indicates the compound was not tested

+ indicates permeability above 1×10⁻⁵ cm/s
++ indicates permeability above 2×10⁻⁶ cm/s
+++ indicates permeability below 2×10⁻⁶ cm/s
NT indicates the compound was not tested

### Example B5. Cytotoxicity Assay

This experiment was designed to obtain a preliminary assessment of cytotoxicity. Compounds were tested at high concentrations to determine if any cytotoxic effects were observed in hepatocyte (HepG2) cell cultures by measuring the release of lactate dehydrogenase (LDH) into the culture media as a measurement of lysed/dead cells.

HepG2 cells were plated in 96-well cell culture plates and allowed to attach overnight at 37° C., 5% CO₂ in EMEM+10% FBS. Treatments were made in complete media (EMEM+10% FBS) and included a dilution series of test compounds from 0.1 to 100 μM. Known cytotoxin cerivastatin was used as a positive assay control and prepared at a final concentration of 0.5 μM.

Growth media was replaced with treatment media (EMEM+10% FBS containing test compound dissolved in DMSO) and cells were incubated with test compounds for 48 hours. At the end of the incubation period, supernatant media from each well was transferred to a new plate and LDH assay working solution was added. LDH assay solution undergoes a colorimetric reaction in proportion to the amount of lactate dehydrogenase (an intracellular protein that was only found in the media in the presence of lysed cells) in the media. Color reaction was quantified by measurement of absorbance at a wavelength of 490 nm.

The signal range of the assay was determined by no manipulation in a negative control treatment and full lysis of all cells in a lysis control sample. Compounds that increase the level of cytotoxicity more than 20% above negative control samples were considered cytotoxic in this assay. Results are shown in Table 6.

### Table 6. Cytotoxicity of Exemplary Compounds.

TABLE 6
Cytotoxicity of Exemplary Compounds.

| Compound | Cytotoxicity | Compound | Cytotoxicity |
|---|---|---|---|
| 1a | ++++ | 2a | ++++ |
| 3a | NT | 4a | ++++ |
| 5a | ++++ | 6a | ++++ |
| 7a | ++++ | 8a | ++++ |
| 9 | ++++ | 10 | NT |
| 11 | ++++ | 12 | ++++ |
| 13 | NT | 14 | ++++ |
| 15 | NT | 16 | NT |
| 17 | ++++ | 18 | NT |
| 19 | ++++ | 20 | ++++ |
| 21 | NT | 22 | ++++ |
| 23 | NT | 24 | NT |
| 25 | NT | 26 | NT |
| 27 | NT | 28 | NT |
| 29 | NT | 30 | ++++ |
| 31 | NT | 32 | NT |
| 33 | NT | 34 | NT |
| 35 | NT | 36 | NT |
| 37 | NT | 38 | NT |
| 39 | NT | 40 | NT |
| 41 | NT | 42 | ++++ |
| 43 | NT | 44 | NT |
| 45 | NT | 46 | NT |
| 47 | NT | 48 | NT |
| 49 | ++++ | 50 | NT |
| 51 | +++ | 52 | NT |
| 53 | +++ | 54 | +++ |

+ indicates non-toxic at 0.1 μM
++ indicates non-toxic at 1 μM
+++ indicates non-toxic at 10 μM
++++ indicates non-toxic at 100 μM
NT indicates the compound was not tested

+ indicates non-toxic at 0.1 μM
++ indicates non-toxic at 1 μM
+++ indicates non-toxic at 10 μM
++++ indicates non-toxic at 100 μM
NT indicates the compound was not tested

### Example B6. In Vitro Stability Assays

Bioavailability can be estimated by compound stability when exposed to conditions in the body. As an initial assessment of stability properties in a variety of conditions present in animals, exemplary compounds were tested for stability in a battery of simulated body compartments. Compounds were tested for stability in the following solutions: simulated gastric fluid (SGF: 34.2 mM NaCl, pH 1.2), simulated gastric fluid with the digestive enzyme pepsin (SGF+Enzyme: SGF with 3.2 mg/ml pepsin), simulated intestinal fluid with the mixture of enzymes in porcine pancreatin (SIF+Enzyme: 28.7 mM NaH₂PO₄, 105.7 mM NaCl, pH 6.8, 10 mg/ml pancreatin), rat plasma, and human plasma.

Test compounds were incubated at a final concentration of 5 μM in the above solutions at 37° C. with samples removed at the following time points: 0, 1, 2, and 4 hours. Reactions were stopped and prepared for quantification by addition of excess quench solution containing an internal standard (acetonitrile, 200 ng/mL bucetin). Test compound and internal standard in each sample was quantified by LC-MS/MS, and after internal normalization to bucetin, test compound concentration was expressed as a percentage of concentration at the 0-hour time point. Stability in the relevant test solution was then determined by the percent remaining at the 4-hour time point. Results are shown in Table 7.

### Table 7. In Vitro Stability of Exemplary Compounds.

TABLE 7
In Vitro Stability of Exemplary Compounds.

| Compound | SGF | SGF + Enz | SIF | Rat Plasma | Human Plasma |
|---|---|---|---|---|---|
| 1a | ++++ | ++++ | ++++ | ++ | ++ |
| 2a | ++++ | +++ | ++++ | ++ | ++ |
| 3a | NT | NT | NT | NT | NT |
| 4a | NT | NT | NT | NT | NT |
| 5a | +++ | + | +++ | ++ | ++ |
| 6a | +++ | + | ++ | +++ | ++ |
| 7a | ++++ | +++ | ++ | NT | NT |
| 8a | ++++ | +++ | +++ | ++++ | ++ |
| 9 | NT | NT | NT | NT | NT |
| 10 | NT | NT | NT | NT | NT |
| 11 | +++ | +++ | ++++ | ++++ | +++ |
| 12 | +++ | +++ | ++ | ++++ | +++ |
| 13 | NT | NT | NT | NT | NT |
| 14 | ++ | ++ | +++ | +++ | +++ |
| 15 | NT | NT | NT | NT | NT |
| 16 | NT | NT | NT | NT | NT |
| 17 | +++ | +++ | +++ | ++ | +++ |
| 18 | NT | NT | NT | NT | NT |
| 19 | +++ | ++ | + | +++ | +++ |
| 20 | +++ | +++ | +++ | ++ | +++ |
| 21 | NT | NT | NT | NT | NT |
| 22 | +++ | +++ | +++ | +++ | +++ |

+ indicates 20-39% compound remaining after 4 hours
++ indicates 40-79% compound remaining after 4 hours
+++ indicates 80-99% compound remaining after 4 hours
++++ indicates 100% compound remaining after 4 hours
NT indicates the compound was not tested

+ indicates 20-39% compound remaining after 4 hours
++ indicates 40-79% compound remaining after 4 hours
+++ indicates 80-99% compound remaining after 4 hours
++++ indicates 100% compound remaining after 4 hours
NT indicates the compound was not tested

### Example B7. In Vivo Pharmacokinetics

Administration of exemplary compounds by selected routes followed by blood collection and compound quantification in plasma was used to determine the pharmacokinetic (PK) profile of the compounds. Compounds were administered to mixed-sex Sprague-Dawley rats of at least 250 grams by dissolving the test compound in DMSO and then diluting the compound into an appropriate vehicle, either saline or saline and poly-ethylene glycol. Dosing was accomplished by either tail vein puncture (IV) or oral gavage (PO), and animals were administered compound according to their weight at 1 mL/kg. At selected intervals following administration (10, 20, 40, 60, 120, and 360 minutes), blood was collected by tail vein blood draw. Whole blood was then processed by centrifugation to produce plasma. Compound content in plasma samples was quantified by LC-MS/MS and compared to an internal standard and standard curves to determine concentration accurately.

Plasma concentrations were then averaged for each time point and plotted as a function of time. Area under the curve was calculated by integration of the curve, Cmax was the highest concentration achieved in plasma, and Tmax was determined by the timing of Cmax. Results are shown in Table 8.

### Table 8. Pharmacokinetic Parameters of Exemplary Compounds.

TABLE 8
Pharmacokinetic Parameters of Exemplary Compounds.

| | | |
|---|---|---|
| AUC | C_{MAX} | T_{MAX} |

| Compound | IV | PO | IV | PO | IV | PO |
|---|---|---|---|---|---|---|
| 1a | ++ | ++ | +++ | + | +++ | ++ |
| 2a | +++ | ++ | +++ | ++ | +++ | ++ |
| 3a | NT | NT | NT | NT | NT | NT |
| 4a | NT | NT | NT | NT | NT | NT |
| 5a | ++ | + | ++ | + | +++ | ++ |
| 6a | +++ | + | +++ | + | +++ | ++ |
| 7a | ++++ | + | ++++ | + | +++ | ++ |
| 8a | NT | NT | NT | NT | NT | NT |
| 9 | NT | NT | NT | NT | NT | NT |
| 10 | NT | NT | NT | NT | NT | NT |
| 11 | +++ | ++ | ++ | ++ | +++ | +++ |
| 12 | ++ | ++ | ++ | + | +++ | + |
| 13 | NT | NT | NT | NT | NT | NT |
| 14 | +++ | +++ | ++ | ++ | +++ | + |
| 15 | NT | NT | NT | NT | NT | NT |
| 16 | NT | NT | NT | NT | NT | NT |
| 17 | NT | NT | NT | NT | NT | NT |
| 18 | NT | NT | NT | NT | NT | NT |
| 19 | NT | NT | NT | NT | NT | NT |
| 20 | +++ | +++ | +++ | ++ | +++ | + |
| 21 | NT | NT | NT | NT | NT | NT |
| 22 | +++ | ++ | ++ | ++ | +++ | ++ |

AUC: ++++ indicates dose-corrected plasma AUC above 3000 ng*h/mL; +++ indicates dose-corrected plasma AUC between 1000-2999 ng*h/mL; ++ indicates dose-corrected plasma AUC between 100-999 ng*h/mL; + indicates dose-corrected plasma AUC between 1-100 ng*h/mL.
Cmax: ++++ indicates dose-corrected plasma Cmax above 3000 ng/mL; +++ indicates dose-corrected plasma Cmax between 1000-2999 ng/mL; ++ indicates dose-corrected plasma Cmax between 100-999 ng/mL; + indicates dose-corrected plasma Cmax between 1-100 ng/mL.
Tmax: +++ indicates Tmax below 30 minutes; ++ indicates Tmax between 30-60 minutes; + indicates Tmax above 60 minutes.
NT indicates the compound was not tested.

AUC: ++++ indicates dose-corrected plasma AUC above 3000 ng*h/mL; +++ indicates dose-corrected plasma AUC between 1000-2999 ng*h/mL; ++ indicates dose-corrected plasma AUC between 100-999 ng*h/mL; + indicates dose-corrected plasma AUC between 1-100 ng*h/mL.
Cmax: ++++ indicates dose-corrected plasma Cmax above 3000 ng/mL; +++ indicates dose-corrected plasma Cmax between 1000-2999 ng/mL; ++ indicates dose-corrected plasma Cmax between 100-999 ng/mL; + indicates dose-corrected plasma Cmax between 1-100 ng/mL.
Tmax: +++ indicates Tmax below 30 minutes; ++ indicates Tmax between 30-60 minutes; + indicates Tmax above 60 minutes.
NT indicates the compound was not tested.

### Example B8. Oral Availability Calculation

Oral bioavailability is critical to developing small molecule therapeutics for oral administration. Calculations of oral bioavailability (% F) are accomplished by comparing in vivo pharmacokinetic data (Example B7) using IV dosing as the maximum possible exposure and determining the exposure rate after PO administration. In these studies, dose corrected AUC from PO administration was divided by dose corrected AUC from IV administration and multiplied by 100 to yield the % F. Results are shown in Table 9.

### Table 9. Calculated Oral Availability of Exemplary Compounds.

TABLE 9
Calculated Oral Availability of Exemplary Compounds.

| Compound | Oral Bioavailability |
|---|---|
| 1a | +++ |
| 2a | +++ |
| 3a | NT |
| 4a | NT |
| 5a | ++ |
| 6a | ++ |
| 7a | + |
| 8a | NT |
| 9 | NT |
| 10 | NT |
| 11 | +++ |
| 12 | +++ |
| 13 | NT |
| 14 | ++++ |
| 15 | NT |
| 16 | NT |
| 17 | NT |
| 18 | NT |
| 19 | NT |
| 20 | ++++ |
| 21 | NT |
| 22 | +++ |

++++ indicates oral bioavailability above 50%
+++ indicates oral bioavailability between 25-50%
++ indicates oral bioavailability between 1-25%
+ indicates oral bioavailability below 1%
NT indicates the compound was not tested

++++ indicates oral bioavailability above 50%
+++ indicates oral bioavailability between 25-50%
++ indicates oral bioavailability between 1-25%
+ indicates oral bioavailability below 1%
NT indicates the compound was not tested

### Example B9. Non-Specific Protein Binding

Plasma and tissue exposures of exemplary compounds were scaled by their non-specific affinity for protein binding in target tissues or fluids to determine the fraction of compound available for interaction with the target. Non-specific binding was determined in blood plasma and brain homogenate collected from mixed-sex Sprague-Dawley rats.

Known concentrations of test compounds were mixed with plasma or brain homogenate and incubated in the donor chamber of a rapid equilibrium dialysis (RED) device with empty PBS buffer in the receiving chamber. After a 4-hour incubation at 37° C. in an orbital shaking incubator, compound in each chamber was quantified by LC-MS/MS. The unbound fraction (f_{u,tissue}) was calculated using the following formula:

$${f}_{u , t i s s u e} = \frac{1}{1 + ( \frac{1}{{f}_{u , h omogenate}} - 1 ) * D}$$

where:
f_{u,tissue} is the unbound fraction in the tissue;
f_{u,homogenate} is the ratio of concentration in the buffer chamber to concentration in the sample chamber; and
D is the dilution factor used to produce the sample.

Results are shown in Table 10.

TABLE 10
Non-Specific Protein Binding of Exemplary Compounds.

Unbound Fraction

| Compound | Plasma | Brain |
|---|---|---|
| 1a | +++ | ++ |
| 2a | +++ | ++ |
| 3a | NT | NT |
| 4a | NT | NT |
| 5a | + | + |
| 6a | ++ | + |
| 7a | ++++ | +++ |
| 8a | NT | NT |
| 9 | NT | NT |
| 10 | NT | NT |
| 11 | + | ++ |
| 12 | ++ | ++ |
| 13 | NT | NT |
| 14 | ++ | ++++ |
| 15 | NT | NT |
| 16 | NT | NT |
| 17 | NT | NT |
| 18 | NT | NT |
| 19 | NT | NT |
| 20 | ++ | +++ |
| 21 | NT | NT |
| 22 | ++ | ++++ |

++++ indicates unbound fraction above 0.9
+++ indicates unbound fraction between 0.5 and 0.9
++ indicates unbound fraction between 0.1 and 0.5
+ indicates unbound fraction below 0.1
NT indicates the compound was not tested

++++ indicates unbound fraction above 0.9
+++ indicates unbound fraction between 0.5 and 0.9
++ indicates unbound fraction between 0.1 and 0.5
+ indicates unbound fraction below 0.1
NT indicates the compound was not tested

### Example B10. In Vivo Tissue Distribution

The rate of distribution to target tissues is an important feature of therapeutic molecules. Tissue distribution of exemplary compounds was performed in mixed-sex Sprague-dawley rats. Test compounds were delivered via tail vein injection (IV) and tissues were collected at Tmax (10 minutes post administration). Animals were deeply anesthetized with isoflurane and whole blood was collected from the right atrium and processed by centrifugation to produce plasma. Animals were then fully perfused with PBS administered to the left ventricle to prevent blood contamination of tissues.

Tissues were collected and homogenized, and compound content in the target tissue was quantified by LC-MS/MS. Tissue distribution rates were determined by dividing the tissue concentration of compound by the plasma concentration and multiplying by 100. Results are shown in Table 11.

TABLE 11
In Vivo Tissue Distribution of Exemplary Compounds.

| | Tissue Distribution (% Plasma exposure at 10 min post IV dose) | | | | | |
|---|---|---|---|---|---|---|
| Compound | Muscle | Sciatic Nerve | Brain | Hippocampus | Cerebellum | Cortex |
| 1a | NT | NT | ++ | ++ | ++ | ++ |
| 2a | ++++ | ++++ | ++ | ++ | + | ++ |
| 3a | NT | NT | NT | NT | NT | NT |
| 4a | NT | NT | NT | NT | NT | NT |
| 5a | NT | NT | ++++ | ++++ | ++++ | ++++ |
| 6a | ++++ | ++++ | ++ | ++ | ++ | ++ |
| 7a | ++ | ++++ | + | + | + | + |
| 8a | NT | NT | NT | NT | NT | NT |
| 9 | NT | NT | NT | NT | NT | NT |
| 10 | NT | NT | NT | NT | NT | NT |
| 11 | NT | ++ | ++ | + | + | + |
| 12 | NT | +++ | ++ | ++ | ++ | ++ |
| 13 | NT | NT | NT | NT | NT | NT |
| 14 | NT | ++ | ++ | ++ | ++ | ++ |
| 15 | NT | NT | NT | NT | NT | NT |
| 16 | NT | NT | NT | NT | NT | NT |
| 17 | NT | NT | NT | NT | NT | NT |
| 18 | NT | NT | NT | NT | NT | NT |
| 19 | NT | NT | NT | NT | NT | NT |
| 20 | NT | ++++ | ++++ | +++ | ++++ | ++++ |
| 21 | NT | NT | NT | NT | NT | NT |
| 22 | NT | +++ | ++ | ++ | ++ | ++ |

++++ indicates distribution above 70%
+++ indicates distribution between 40 and 69%
++ indicates distribution between 5 and 39%
+ indicates distribution between 0.05 and 5%
NT indicates the compound was not tested

++++ indicates distribution above 7000
+++ indicates distribution between 40 and 690%
++ indicates distribution between 5 and 390%
+ indicates distribution between 0.05 and 50%
NT indicates the compound was not tested Example Bit. In Vivo Efficacy: Scopolamine-Induced Spatial Memory Deficit in the Morris Water Maze.

Exemplary compounds 2a and 6a were evaluated for their ability to reverse chemically-induced spatial memory deficits in rats in the Morris water maze. The water maze consists of a large round tank (diameter 2.1 m) filled with 26-28° C. water to a depth of −30 cm and the water was clouded with white paint. A round platform (13 cm diameter) was fixed such that it rested 2-3 cm below the surface of the water. High-contrast visual cues were placed around the tank to aid spatial orientation of test animals. Testing consisted of placing an animal into the water facing the tank wall at one of three randomly assigned starting locations and allowing the animal to swim and search for the hidden platform for up to 120 seconds. The time taken for the animal to locate the platform was recorded as the escape latency. Animals were tested 5 times per day with a 30 second rest period between trials. Testing was completed for a total of 8 consecutive days.

Animals were divided into groups (N=8 per group) depending on treatment. Control animals received only empty vehicle. Scopolamine groups received 3 mg/kg scopolamine dissolved in sterile saline by intraperitoneal (IP) injection 30 minutes prior to testing. Test compound groups received test compound at various concentrations by oral gavage (PO) dissolved in 48% sterile saline, 50% polyethylene glycol (PEG-400), and 2% DMSO 40 minutes prior to testing. Escape latencies were recorded for each animal for 5 trials each day for 8 consecutive days. Changes in escape latency curves were statistically analyzed by 2-way ANOVA with Bonferoni post-test. Results are shown in Table 12.

Exemplary compound 1a was evaluated for its ability to reverse chemically-induced spatial memory deficits in rats in the Morris water maze. The water maze consists of a large round tank (diameter 1.5 m) filled with 23-26° C. water to a depth of −30 cm and the water was clouded with white paint. A round platform was fixed such that it rested 2-3 cm below the surface of the water. High-contrast visual cues were placed around the tank to aid spatial orientation of test animals. Testing consisted of placing an animal into the water facing the tank wall at one of three randomly assigned starting locations and allowing the animal to swim and search for the hidden platform for up to 90 seconds. The time taken for the animal to locate the platform was recorded as the escape latency. Animals were tested 5 times per day with a 30 second rest period between trials. Testing was completed for a total of 5 consecutive days.

Animals were divided into groups (N=12 per group) depending on treatment. Control animals received only empty vehicle. Scopolamine groups received 2 mg/kg scopolamine dissolved in sterile saline by intraperitoneal (IP) injection 30 minutes prior to testing. Test compound groups received test compound at various concentrations by oral gavage (PO) dissolved in 78% sterile saline, 20% polyethylene glycol (PEG-400), and 2% DMSO 40 minutes prior to testing. Escape latencies were recorded for each animal for 5 trials each day for 5 consecutive days. Changes in escape latency curves were statistically analyzed by 2-way ANOVA with Bonferoni post-test. Results are shown in Table 12.

TABLE 12
In Vivo Efficacy of Exemplary Compounds.

| Compound | Dose (mg/kg) | Cognitive Improvement |
|---|---|---|
| 1a | 8 | − |
| | 2 | − |
| | 0.5 | + |
| 2a | 10 | − |
| | 1 | +++ |
| | 0.1 | − |
| 6a | 10 | ++ |
| | 1 | − |

+++ indicates post-test p-value below 0.01
++ indicates post-test p-value between 0.05 and 0.01
+ indicates post-test p-value between 0.06 and 0.05
− indicates post-test p-value above 0.06

+++ indicates post-test p-value below 0.01
++ indicates post-test p-value between 0.05 and 0.01
+ indicates post-test p-value between 0.06 and 0.05
indicates post-test p-value above 0.06

### Example B12. Broad Protection from Neurotoxicity by Compound 1a In Vitro

HGF/MET signaling activation is expected to protect cells, including neurons, from cell death. To demonstrate the HGF/MET augmentation properties of example compounds, we tested the ability of compound 1a to improve viability of cultured primary neurons subjected to chemical insults that produce neuron death through a range of mechanisms. Hydrogen peroxide (H₂O₂) treatment produces toxic oxidative stress. Bacteria-derived lipopolysaccharide (LPS) treatment induces inflammatory cell death. Treatment with excess glutamate (Glut) produces excitotoxicity in neuron cultures. Treatment with 1-methyl-4-phenylpyridinium (MPP⁺) causes cell death by inhibition of mitochondrial function.

Rat primary cortical neuron cultures were grown at 37° C., 5% CO₂ in Complete Neurobasal Medium supplemented with B27/GDNF/BDNF containing 10% FBS until maturity and were then seeded in 384-well plate at 5,000 cells/well. Cultures were then treated for 15 minutes with 1 uM, 100 nM, 10 nM, 1 nM, or 0.1 nM of compound 1a. Cells were then subjected to individual conditional insults for 24 hours at the following concentrations: H₂O₂ at 1 uM, LPS at 1 uM, glutamate at 25 uM, MPP+ at 500 uM. All treatments contained recombinant human HGF (R&D Systems) at a concentration of 5 ng/ml.

To quantify neuroprotection, cell viability was measured with the Cell Titer-Glo Luminescent Cell Viability Assay (Promega, Cat #G7571). Data was normalized to DMSO+HGF control set at 100% cell viability. Statistical analyses for each group (n=4) were performed using one-way ANOVA with Tukey's Multiple comparison test.

The results are shown in Table 13. Data is reported as statistical significance vs. insult control, with NS=not significant, +=p<0.05, ++=p<0.01, and +++=p<0.001.

TABLE 13
Compound 1a protects rat primary neurons from a variety of neurotoxic insults

| | | | | | | | Positive |
|---|---|---|---|---|---|---|---|
| | Insult effect | | | | | | Control |
| | (% viability | [compound 1a] + HGF (5 ng/ml) | | | | | MK-801 |
| Insult | vs vehicle) | 1 uM | 100 nM | 100 nM | 1 nM | 0.1 nM | (1 uM) |
| H₂O₂ (1 uM) | 64.23 | +++ | +++ | +++ | +++ | +++ | +++ |
| Glutamate (25 uM) | 49.55 | +++ | +++ | +++ | +++ | +++ | +++ |
| LPS (1 uM) | 49.19 | +++ | +++ | +++ | NS | +++ | +++ |
| MPP+ (500 uM) | 49.85 | +++ | +++ | +++ | +++ | +++ | +++ |

In this study, compound 1a treatment acted as an effective neuroprotective treatment at a broad range of concentrations against the entire cohort of insults tested. This result indicates that augmentation of HGF/MET signaling with compound 1a exerted a potent neuroprotective effect on rat neurons in culture.

### Example B13: Attenuation of Scopolamine-Induced Cognitive Impairment

Mild cognitive impairment can be assessed in rodent models via delivery of various compounds that interfere with normal neurotransmitter signaling. Scopolamine is a compound that is well-known to antagonize muscarinic acetylcholine receptors, nicotinic receptors, as well as glutamatergic receptors such as NMDA receptors. Thus, delivery of scopolamine results in cognitive impairment, which can be assessed in a variety of cognitive tasks. Positive modulation of the HGF/MET system can promote NMDA receptor activation and results in pro-cognitive effects. Two test compounds, Compound 1a and Compound 5a, were evaluated for their ability to protect against scopolamine-induced cognitive impairment.

In this study, mild cognitive impairment was modeled in otherwise healthy male CD-1 mice (4-5 weeks old at study start) by acute delivery of 0.89 mg/kg scopolamine (dissolved in saline). Test compounds were administered via oral gavage 0.5 hours prior to scopolamine exposure, and cognitive performance was assessed in the T-maze spontaneous alternation task 0.5 hours after scopolamine exposure (1 hour following test compound exposure). The experimental protocol consisted of one single session, which started with 1 “forced-choice” trial, followed by 14 “free-choice” trials. In the first “forced-choice” trial, the animal was confined 5 seconds in the start arm and was then released while either the left or right goal arm was blocked by a horizontal door. After the mouse entered the open goal arm and returned to the start position, the left or right goal door was opened, and the animal was allowed to choose freely between the left and right goal arm (“free choice trials”). The animal was considered to have entered into an arm when it placed its four paws in the arm. Normal animals will choose to explore the previously-unexplored arm, which is recorded as a spontaneous alternation. Exploring the previously-entered arm indicates that the animal no longer remembers which arm was previously explored, and is considered a cognitive deficit behavior. The session was terminated, and the animal was removed from the maze as soon as 14 free-choice trials had been performed or 10 min had elapsed, whichever event occurred first.

Variables analyzed included percent of spontaneous alternations (formula) and results are shown in Table 14. This percentage was defined as entry into a different goal arm of the T maze over successive trials (e.g., left-right-left-right) and was calculated using the formula: (% alternations=Number of Alternations/14*100). Both compounds attenuated the degree of scopolamine-induced cognitive impairment, as indicated by increased percent of spontaneous alternations compared to scopolamine-only controls. Statistical analysis was accomplished via 1-way ANOVA with Dunnet's multiple comparisons post-test.

TABLE 14
Scopolamine-induced cognitive impairment in T-Maze task

| | | | Compound 1a | | | | | Compound 5a | | | | |
|---|---|---|---|---|---|---|---|---|---|---|---|---|
| | Vehicle | SCO | (mg/kg via PO) | | | | | (mg/kg via PO) | | | | |
| Dose | | 0.89 | 0.5 | 2 | 8 | 16 | 32 | 1.25 | 2.5 | 5 | 10 | 20 |
| Spontaneous | 69 | 37 | 52 | 55 | 47 | 51 | 55 | 51 | 57 | 52 | 55 | 52 |
| alternations % | | | | | | | | | | | | |
| Significance | | | ** | ** | ns | * | ** | * | ** | * | ** | * |
| (compared to | | | | | | | | | | | | |
| scopolamine | | | | | | | | | | | | |
| only) | | | | | | | | | | | | |

* = p < 0.05,
** = p < 0.01,
ns = not significant

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A method of treating mild cognitive impairment in a subject in need thereof, comprising administering an effective amount of a compound of Formula (I): 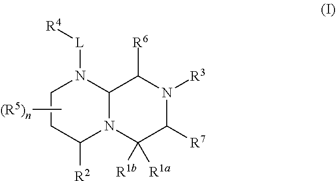
or a pharmaceutically acceptable salt thereof, wherein:
L is a direct bond, —C(═O)—, —(CR^{a}R^{b})ₘ—C(═O)—, —C(═O)—(CR^{a}R^{b})ₘ—, or —(CR^{a}R^{b})ₘ—;
each Rₐ and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

2. The method of claim 1, wherein L is —C(═O)— or —(CR^{a}R^{b})ₘ—.

3. The method of claim 1 or 2, wherein L is a —C(═O)—.

4. The method of claim 1 or 2, wherein L is —(CR^{a}R^{b})ₘ—.

5. The method of claim 4, wherein Rₐ and R^{b} are each H, and m is 1.

6. The method of any one of claims 1-5, wherein R^{1a} and R^{1b} are each independently H; C₁-C₆ alkyl optionally substituted with 1-3 substituents selected from halo, —CO₂H, and —C(═O)NH₂; C₁-C₆ alkoxy; halo; or C₆-C₁₀ arylalkyl optionally substituted by 1-3 substituents selected from halo and amino.

7. The method of claim 6, wherein R^{1a} and R^{1b} are each independently H, methyl, fluoro, 2-methylbutyl, —CH₂F, methoxy, —CH₂CO₂H, —CH₂C(═O)NH₂, benzyl, or 4-aminobenzyl.

8. The method of claim 6, wherein R^{1a} and R^{1b} are each independently H or C₁-C₃ alkyl.

9. The method of claim 8, wherein R^{1a} is methyl and R^{1b} is H.

10. The method of claim 8, wherein R^{1a} and R^{1b} are each H.

11. The method of any one of claims 1-10, wherein R² is H.

12. The method of any one of claims 1-10, wherein R² is thioxo.

13. The method of any one of claims 1-10, wherein R² is oxo.

14. The method of any one of claims 1-13, wherein R³ is C₃-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl, wherein the alkyl, alkenyl, alkynyl, cycloalkyl, cycloalkylalkyl, arylalkyl, heteroarylalkyl, or heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

15. The method of any one of claims 1-13, wherein R³ is C₂-C₆ alkyl optionally substituted by 1-3 substituents selected from halo, C₁-C₃ alkoxy, hydroxy, —NH₂, —SO₂(C₁-C₃ alkyl), and —C(═O)NH₂; C₂-C₆ alkenyl; C₃-C₆ cycloalkylalkyl; 5- to 6-membered heteroarylalkyl; 5- to 6-membered heterocyclylalkyl; or C₆ arylalkyl.

16. The method of claim 15, wherein R³ is C₂ alkyl substituted by 1-3 substituents selected from C₁-C₃ alkoxy, hydroxy, —NH₂, and —SO₂(C₁-C₃ alkyl).

17. The method of any one of claims 14-16, wherein R³ is:

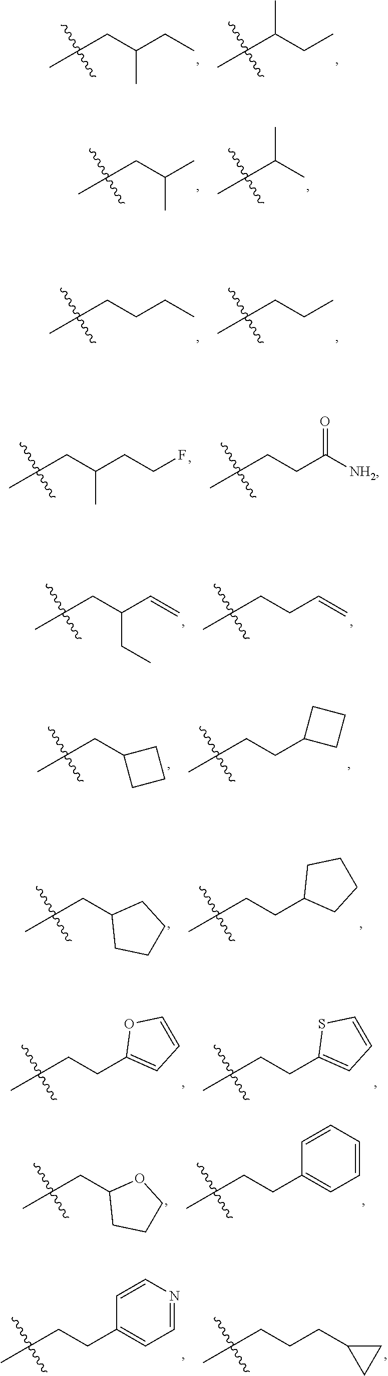

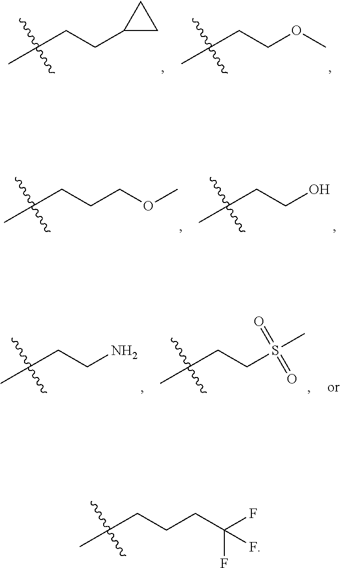

18. The method of claim 17, wherein R³ is:

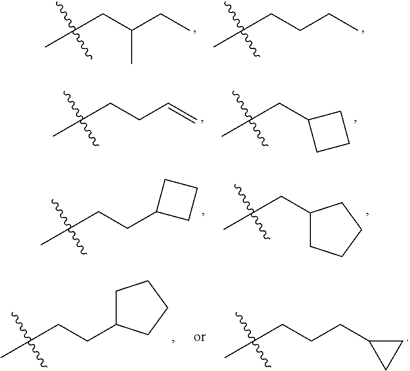

19. The method of any one of claims 1-18, wherein R⁴ is C₆-C₁₀ aryl optionally substituted with 1-3 substituents selected from halo, hydroxyl, C₁-C₆ haloalkyl, and C₁-C₆ haloalkoxy.

20. The method of claim 19, wherein R⁴ is phenyl substituted with 1-3 substituents selected from —CF₃, —OCHF₂, —OH, fluoro, and chloro.

21. The method of claim 20, wherein R⁴ is:

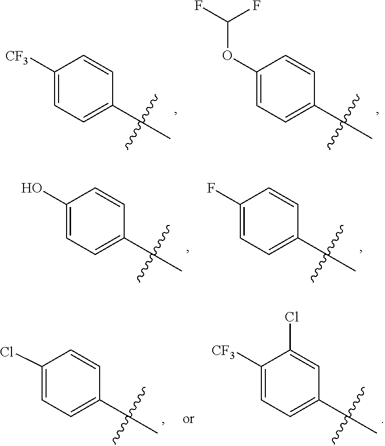 [SMILES: *c1ccc(C(F)(F)F)cc1 ; *c1ccc(OC(F)F)cc1 ; *c1ccc(O)cc1 ; *c1ccc(F)cc1 ; *c1ccc(Cl)cc1 ; *c1ccc(C(F)(F)F)c(Cl)c1]

22. The method of claim 21, wherein R⁴ is:

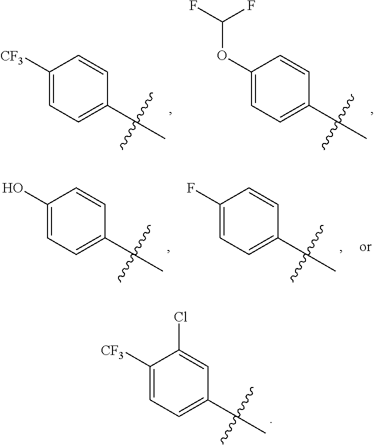 [SMILES: *c1ccc(C(F)(F)F)cc1 ; *c1ccc(OC(F)F)cc1 ; *c1ccc(O)cc1 ; *c1ccc(F)cc1 ; *c1ccc(C(F)(F)F)c(Cl)c1]

23. The method of any one of claims 1-18, wherein R⁴ is 5- to 10-membered heteroaryl optionally substituted with 1-3 substituents selected from halo, hydroxyl, C₁-C₆ haloalkyl, and C₁-C₆ haloalkoxy.

24. The method of claim 23, wherein
R⁴ is pyridyl or indolyl optionally substituted with 1-3 substituents selected from halo, hydroxyl, C₁-C₆ haloalkyl, and C₁-C₆ haloalkoxy.

25. The method of claim 24, wherein
R⁴ is

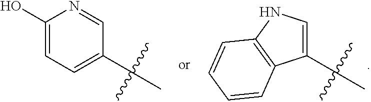 [SMILES: *c1ccc(O)nc1 ; *c1c[nH]c2ccccc12]

26. The method of claim 25, wherein
R⁴ is

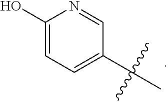 [SMILES: *c1ccc(O)nc1]

27. The method of any one of claims 1-18, wherein R⁴ is 5- to 10-membered heterocyclyl optionally substituted with 1-3 substituents selected from halo, hydroxyl, C₁-C₆ haloalkyl, and C₁-C₆ haloalkoxy.

28. The method of claim 27, wherein R⁴ is indolinyl.

29. The method of claim 28, wherein R⁴ is

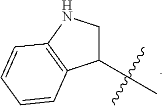 [SMILES: *C1CNc2ccccc21]

30. The method of any one of claims 1-26, wherein -L-R⁴ is:

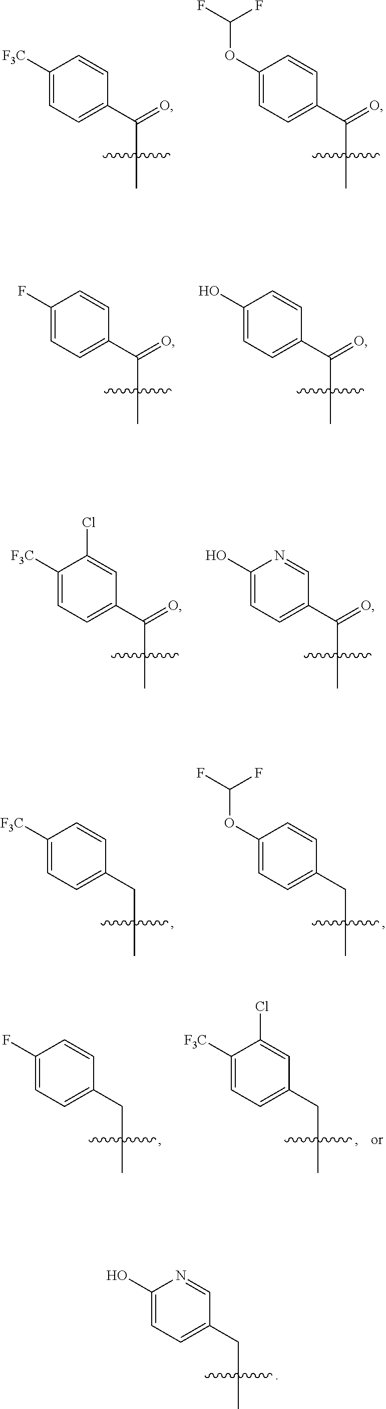

31. The method of any one of claims 1-30, wherein n is 0.

32. The method of any one of claims 1-30, wherein n is 1.

33. The method of claim 32, wherein R⁵ is oxo or halo.

34. The method of claim 33, wherein R⁵ is oxo or fluoro.

35. The method of any one of claims 1-34, wherein R⁶ is H.

36. The method of any one of claims 1-35, wherein R⁷ is oxo.

37. The method of any one of claims 1-10, 13-31, 35, and 36, wherein the compound is of Formula (V): 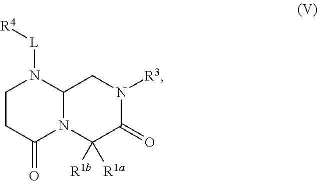
or a pharmaceutically acceptable salt thereof.

38. The method of claim 37, wherein:
L is —C(═O)— or —CH₂—;
R^{1a} and R^{1b} are independently H or C₁-C₃ alkyl optionally substituted with —CO₂H;
R³ is C₄-C₅ alkyl, C₄-C₅ alkenyl, or C₁-C₃ alkyl substituted with C₃-C₅ cycloalkyl; and
R⁴ is phenyl or pyridyl substituted with 1-3 substituents selected from —CF₃, —OCHF₂, —OH, fluoro, and chloro.

39. A method of treating mild cognitive impairment in a subject in need thereof, comprising administering an effective amount of a compound selected from the compounds of Table 1A and pharmaceutically acceptable salts thereof.

40. The method of any one of the preceding claims, wherein the method slows progression of dementia in the subject.

41. The method of any one of the preceding claims, wherein the method improves cognitive function or slows progression of cognitive dysfunction in the subject.

42. The method of any one of the preceding claims, wherein the compound is administered by oral or intravenous administration.
